(19) United States
(12) Patent Application Publication
(10) Pub. No.: US 2024/0042019 A1
(43) Pub. Date: Feb. 8, 2024

(54) OPTIMIZED TLR7 LIGANDS AND USES THEREOF

(71) Applicant: Genentech, Inc., South San Francisco, CA (US)

(72) Inventors: Emily Crane FREUND, San Mateo, CA (US); Anna-Maria HERZNER, Mittelbiberach (DE); Rebecca Ani LEYLEK, Redwood City, CA (US); Ann-Jay TONG, Alameda, CA (US); Sara Marie WICHNER, San Francisco, CA (US); Lélia Yvonne DELAMARRE, San Francisco, CA (US)

(21) Appl. No.: 18/485,987

(22) Filed: Oct. 12, 2023

Related U.S. Application Data
(63) Continuation of application No. PCT/US2022/025128, filed on Apr. 15, 2022.
(60) Provisional application No. 63/176,128, filed on Apr. 16, 2021.

(51) Int. Cl. A61K 39/39; C12N 15/113; A61P 37/02

(52) U.S. Cl. CPC A61K 39/39; C12N 15/1138; A61P 37/02; C12N 2310/315; C12N 2310/321; C12N 2310/531

(57) ABSTRACT

The present disclosure relates to immunomodulatory compositions that selectively activate TLR7, comprising an RNA ligand and a pharmaceutically acceptable carrier, as well as to in vitro and therapeutic uses of such immunomodulatory compositions.

### CROSS-REFERENCE TO RELATED APPLICATIONS

This application is a continuation of International Application No. PCT/US2022/025128, filed Apr. 15, 2022, which claims the benefit of U.S. Provisional Application No. 63/176,128, filed Apr. 16, 2021, the entire contents of each of which are hereby incorporated by reference in their entireties.

### REFERENCE TO AN ELECTRONIC SEQUENCE LISTING

The content of the electronic sequence listing (146392053201seglist.xml; Size: 120,532 bytes; and Date of Creation: Oct. 11, 2023) is herein incorporated by reference in its entirety.

### FIELD

The present disclosure relates to immunomodulatory compositions and uses thereof.

### BACKGROUND

Nucleic acid receptors of the innate immune system are important sentinels to guard the human host from viral infection. The cytosol and endosomes are the two main compartments of the cell that play major roles in sensing viral RNA. In the cytosol, RIG-I-like receptors, which are expressed in almost every cell type, sense double-stranded RNA produced as replication byproducts. In the endosomes, Toll-like receptors (TLRs) such as TLR7 and TLR8, which are mostly expressed by specialized immune cell types called antigen presenting cells (i.e., plasmacytoid dendritic cells (pDCs), conventional dendritic cells (cDCs), myeloid cells, and B cells), detect degradation products of single-stranded RNAs (ssRNAs). Such RNA degradation products may be derived from phagocytosis of viruses, virus-infected cells, necrotic cells, cancerous tissues, RNA-containing antibody complexes, or liposomal formulations of chemically synthesized or in vitro transcribed RNA.

The activation of Toll-like receptors such as TLR7 and TLR8 on innate immune cells is critical to induce inflammation and to initiate the adaptive immune response. While little is known about the enzymes involved in RNA degradation for TLR7 activation, endosomal RNase T2 and RNase 2 are required for TLR8 activation.

The pattern of expression of TLR7 and TLR8 differs. TLR7 is expressed and functional in both mice and humans, while TLR8, although expressed in both humans and mice, is only functional in human cells. TLR7 is expressed by all antigen-presenting cells, including myeloid cells, in mice, but its expression is restricted to pDCs and B cells in humans. In contrast, TLR8 is expressed in myeloid cells including cDCs, monocytes and macrophages in humans.

In addition to their differences in expression patterns, TLR7 and TLR8 induce distinct cytokine profiles upon activation. Stimulation of TLR8 on human monocytes or cDCs leads to activation of a broad range of inflammatory cytokines such as TNFα and IL-6 in an NF-kB-dependent manner. On the other hand, stimulation of human pDCs through TLR7 induces limited NF-kB-dependent inflammatory cytokines and large amounts of type I IFNs, such as IFNα.

Type I IFNs are a key family of cytokines with roles in antiviral immunity, promoting adaptive immunity, and in anti-tumor immunity. In particular, the role of type I IFNs in anti-tumor immunity is achieved by activation of cDCs, which results in IL-12 secretion, increased antigen presentation, and enhanced antigen-specific CD8 T-cell responses. Furthermore, TLR7 agonists have been shown to synergize with cancer immunotherapies such as PD-L1-targeted therapies by activating cDCs to initiate and increase T-cell priming in immunosuppressive tumor environments in mice.

The unique cytokine profile induced by TLR7 activation, and the limited expression of TLR7 in humans (i.e., in pDCs and B cells) in comparison to TLR8 suggest that TLR7-selective ligands could avoid some of the toxicity that has been associated with dual TLR7/8 agonists, including RNA-based therapeutics. However, while multiple TLR8-selective ligands have been described, therapeutic TLR7-selective RNA ligands are very rare.

Accordingly, there is a need in the art for RNA ligands that have an enhanced ability to selectively activate TLR7.

All references cited herein, including patent applications and publications, are hereby incorporated by reference in their entirety.

### SUMMARY

In some aspects, provided herein is an immunomodulatory composition that selectively activates TLR7, comprising an RNA ligand and a pharmaceutically acceptable carrier, wherein the RNA ligand comprises: (a) a TLR7-selective motif comprising Formula II: C₁*U₂-U₃*U₄-U₅*C₆ (Formula II), wherein C₁ and C₆ are cytidine nucleosides, and U₂, U₃, U₄, and U₅ are uridine nucleosides, wherein U₂ has a 2′O-methyl modification (fU), a 2′-fluoro modification (fU), a 2′-amino modification, a 2′-deoxy modification, or a 2′-methoxyethoxy modification, wherein U₄ has a 2′O-methyl modification (mU), a 2′-fluoro modification (fU), a 2′-amino modification, a 2′-deoxy modification, or a 2′-methoxyethoxy modification, and wherein “*” represents a phosphorothioate linkage and “-” represents a phosphodiester linkage; (b) a first nucleotide sequence linked to the 5′ end of the TLR7-selective motif; and (c) a second nucleotide sequence linked to the 3′ end of the TLR7-selective motif. In some embodiments, U₂ of the TLR7-selective motif has a 2′O-methyl modification (mU). In some embodiments, U₄ of the TLR7-selective motif has a 2′O-methyl modification (mU). In some embodiments, U₂ of the TLR7-selective motif has a 2′-fluoro modification (RU). In some embodiments, U₄ of the TLR7-selective motif has a 2′-fluoro modification (RU). In some embodiments, U₂ and U₄ of the TLR7-selective motif have a 2′O-methyl modification (mU). In some embodiments, U₂ and U₄ of the TLR7-selective motif have a 2′-fluoro modification (RI). In some embodiments, U₂ of the TLR7-selective motif has a 2′O-methyl modification (mU), and U₄ of the TLR7-selective motif has a 2′-fluoro modification (RI). In some embodiments, U₂ of the TLR7-selective motif has a 2′-fluoro modification (RI), and U₄ of the TLR7-selective motif has a 2′O-methyl modification (mU). In some embodiments, the TLR7-selective motif comprises the nucleotide sequence of SEQ ID NO: 44. In some embodiments, the TLR7-selective motif comprises the nucleotide sequence of SEQ ID NO: 45. In some embodiments, the TLR7-selective motif comprises the nucleotide sequence of SEQ ID NO: 46. In some embodiments, the TLR7-selective motif comprises the nucleotide sequence of SEQ ID NO: 47.

In another aspect, provided herein is an immunomodulatory composition that selectively activates TLR7, comprising an RNA ligand and a pharmaceutically acceptable carrier, wherein the RNA ligand comprises: (a) a TLR7-selective motif comprising a nucleotide sequence selected from C*mU-U*mU-U*C, C*fU-U*fU-U*C, C*mU-U*fU-U*C, or C*fU-U*mU-U*C,
(b) a first nucleotide sequence linked to the 5′ end of the TLR7-selective motif, and (c) a second nucleotide sequence linked to the 3′ end of the TLR7-selective motif.

In some embodiments, which may be combined with any of the preceding aspects or embodiments, the first nucleotide sequence comprises one nucleoside, or at least two nucleosides linked by phosphorothioate linkages, wherein the first nucleotide sequence does not comprise a uridine nucleoside, and wherein the first nucleotide sequence is linked to the TLR7-selective motif by a phosphorothioate linkage. In some embodiments, the first nucleotide sequence comprises eight nucleosides linked by phosphorothioate linkages. In some embodiments, the first nucleotide sequence comprises the nucleotide sequence C*C*G*A*G*C*C*G, or a nucleotide sequence having at least about 85% identity to SEQ ID NO: 58, wherein “*” represents a phosphorothioate linkage, and wherein the first nucleotide sequence is linked to the TLR7-selective motif by a phosphorothioate linkage.

In some embodiments, which may be combined with any of the preceding aspects or embodiments, the second nucleotide sequence comprises one nucleoside, or at least two nucleosides linked by phosphorothioate linkages, wherein the second nucleotide sequence does not comprise a uridine nucleoside, and wherein the second nucleotide sequence is linked to the TLR7-selective motif by a phosphorothioate linkage. In some embodiments, the second nucleotide sequence comprises two nucleosides linked by phosphorothioate linkages. In some embodiments, the second nucleotide sequence comprises the nucleotide sequence C*C, wherein “*” represents a phosphorothioate linkage, and wherein the second nucleotide sequence is linked to the TLR7-selective motif by a phosphorothioate linkage.

In some embodiments, which may be combined with any of the preceding aspects or embodiments, the RNA ligand comprises the nucleotide sequence of SEQ ID NO: 12, or a nucleotide sequence having at least about 85% identity to SEQ ID NO: 12 and comprising the TLR7-selective motif of SEQ ID NO: 44. In some embodiments, which may be combined with any of the preceding aspects or embodiments, the RNA ligand comprises the nucleotide sequence of SEQ ID NO: 13, or a nucleotide sequence having at least about 85% identity to SEQ ID NO: 13 and comprising the TLR7-selective motif of SEQ ID NO: 45. In some embodiments, which may be combined with any of the preceding aspects or embodiments, the RNA ligand comprises the nucleotide sequence of SEQ ID NO: 14, or a nucleotide sequence having at least about 85% identity to SEQ ID NO: 14 and comprising the TLR7-selective motif of SEQ ID NO: 46. In some embodiments, which may be combined with any of the preceding aspects or embodiments, the RNA ligand comprises the nucleotide sequence of SEQ ID NO: 15, or a nucleotide sequence having at least about 85% identity to SEQ ID NO: 15 and comprising the TLR7-selective motif of SEQ ID NO: 47.

In another aspect, provided herein is an immunomodulatory composition that selectively activates TLR7, comprising an RNA ligand and a pharmaceutically acceptable carrier, wherein the RNA ligand comprises: (a) a nucleotide sequence selected from SEQ ID NOs: 12, 13, 14, or 15; (b) a nucleotide sequence having at least about 85% identity to SEQ ID NO: 12 and comprising the TLR7-selective motif of SEQ ID NO: 44; (c) a nucleotide sequence having at least about 85% identity to SEQ ID NO: 13 and comprising the TLR7-selective motif of SEQ ID NO: 45; (d) a nucleotide sequence having at least about 85% identity to SEQ ID NO: 14 and comprising the TLR7-selective motif of SEQ ID NO: 46; or (e) a nucleotide sequence having at least about 85% identity to SEQ ID NO: 15 and comprising the TLR7-selective motif of SEQ ID NO: 47.

In another aspect, provided herein is an immunomodulatory composition that selectively activates TLR7, comprising an RNA ligand and a pharmaceutically acceptable carrier, wherein the RNA ligand comprises: (a) a TLR7-selective motif comprising Formula III: C₁—U₂-C₃ (Formula III), wherein C₁ and C₃ are cytidine nucleosides and U₂ is a uridine nucleoside, wherein C₁ has a 2′O-methyl modification (mU), a 2′-fluoro modification (fU), a 2′-amino modification, a 2′-deoxy modification, or a 2′-methoxyethoxy modification, and wherein “-” represents a phosphodiester linkage; (b) a first nucleotide sequence linked to the 5′ end of the TLR7-selective motif; and (c) a second nucleotide sequence linked to the 3′ end of the TLR7-selective motif. In some embodiments, C₁ of the TLR7-selective motif has a 2′-fluoro modification (fC). In some embodiments, C₁ of the TLR7-selective motif has a 2′O-methyl modification (mC). In some embodiments, the TLR7-selective motif comprises the nucleotide sequence of SEQ ID NO: 42. In some embodiments, the TLR7-selective motif comprises the nucleotide sequence of SEQ ID NO: 43.

In another aspect, provided herein is an immunomodulatory composition that selectively activates TLR7, comprising an RNA ligand and a pharmaceutically acceptable carrier, wherein the RNA ligand comprises: (a) a TLR7-selective motif comprising the nucleotide sequence of fC-U-C or mC-U-C, (b) a first nucleotide sequence linked to the 5′ end of the TLR7-selective motif, and (c) a second nucleotide sequence linked to the 3′ end of the TLR7-selective motif.

In some embodiments, which may be combined with any of the preceding aspects or embodiments, the RNA ligand is capable of adopting a double-stranded RNA hairpin structure. In some embodiments, which may be combined with any of the preceding aspects or embodiments, the RNA ligand comprises a G:U wobble base pair.

In some embodiments, which may be combined with any of the preceding aspects or embodiments, the first nucleotide sequence does not comprise a uridine nucleoside, and wherein the first nucleotide sequence is linked to the TLR7-selective motif by a phosphodiester linkage.

In some embodiments, the first nucleotide sequence comprises three nucleosides. In some embodiments, the nucleosides within the first nucleotide sequence are linked by phosphodiester linkages. In some embodiments, the first nucleotide sequence comprises the nucleotide sequence C-G-G, wherein “-” represents a phosphodiester linkage.

In some embodiments, which may be combined with any of the preceding aspects or embodiments, the second nucleotide sequence does not comprise a uridine nucleoside, wherein the second nucleotide sequence comprises the nucleotide sequence G-G-G, wherein the nucleotide sequence G-G-G is capable of base pairing with the TLR7-selective motif, and wherein the second nucleotide sequence is linked to the TLR7-selective motif by a phosphodiester linkage. In some embodiments, the second nucleotide sequence comprises about 15 nucleosides. In some embodiments, the nucleosides within the second nucleotide sequence are linked by phosphodiester linkages. In some embodiments, the second nucleotide sequence comprises: (a) the nucleotide sequence G-G-C-A-G-A-A-G-C-C-G-G-G-C-C(SEQ ID NO: 61), or (b) a nucleotide sequence having at least about 85% identity to SEQ ID NO: 61 and comprising the nucleotide sequence G-G-G, wherein the nucleotide sequence G-G-G is capable of base pairing with the TLR7-selective motif, and wherein “-” represents a phosphodiester linkage.

In some embodiments, which may be combined with any of the preceding aspects or embodiments, the RNA ligand comprises: (a) the nucleotide sequence of SEQ ID NO: 23, or (b) a nucleotide sequence having at least about 85% identity to SEQ ID NO: 23 and comprising the TLR7-selective motif of SEQ ID NO: 42 and the nucleotide sequence G-G-G, wherein the nucleotide sequence G-G-G is capable of base pairing with the TLR7-selective motif. In some embodiments, which may be combined with any of the preceding aspects or embodiments, the RNA ligand comprises: (a) the nucleotide sequence of SEQ ID NO: 24, or (b) a nucleotide sequence having at least about 85% identity to SEQ ID NO: 24 and comprising the TLR7-selective motif of SEQ ID NO: 43 and the nucleotide sequence G-G-G, wherein the nucleotide sequence G-G-G is capable of base pairing with the TLR7-selective motif.

In another aspect, provided herein is an immunomodulatory composition that selectively activates TLR7, comprising an RNA ligand and a pharmaceutically acceptable carrier, wherein the RNA ligand comprises: (a) a nucleotide sequence of SEQ ID NOs: 23 or 24; (b) a nucleotide sequence having at least about 85% identity to SEQ ID NO: 23 and comprising the TLR7-selective motif of SEQ ID NO: 42 and the nucleotide sequence G-G-G, wherein the nucleotide sequence G-G-G is capable of base pairing with the TLR7-selective motif; or (c) a nucleotide sequence having at least about 85% identity to SEQ ID NO: 24 and comprising the TLR7-selective motif of SEQ ID NO: 43 and the nucleotide sequence G-G-G, wherein the nucleotide sequence G-G-G is capable of base pairing with the TLR7-selective motif.

In another aspect, provided herein is an immunomodulatory composition that selectively activates TLR7, comprising an RNA ligand and a pharmaceutically acceptable carrier, wherein the RNA ligand comprises: (a) a first and a second TLR7-selective motif comprising Formula IV: C₁—U₂-U₃-C₄ (Formula IV), wherein C₁ and C₄ are cytidine nucleosides and U₂ and U₃ are uridine nucleosides, wherein U₂ has a 2′O-methyl modification (mU), a 2′-fluoro modification (fU), a 2′-amino modification, a 2′-deoxy modification, or a 2′-methoxyethoxy modification, and wherein “-” represents a phosphodiester linkage; (b) a first nucleotide sequence linked to the 5′ end of the first TLR7-selective motif; and (c) a second nucleotide sequence linked to the 3′ end of the first TLR7-selective motif and to the 5′ end of the second TLR7-selective motif. In some embodiments, U₂ of the first TLR7-selective motif has a 2′O-methyl modification (mU). In some embodiments, U₂ of the second TLR7-selective motif has a 2′O-methyl modification (mU). In some embodiments, U₂ of the first TLR7-selective motif has a 2′-fluoro modification (fU). In some embodiments, U₂ of the second TLR7-selective motif has a 2′-fluoro modification (fU). In some embodiments, the first TLR7-selective motif comprises the nucleotide sequence of SEQ ID NO: 36. In some embodiments, the second TLR7-selective motif comprises the nucleotide sequence of SEQ ID NO: 36. In some embodiments, the first TLR7-selective motif comprises the nucleotide sequence of SEQ ID NO: 37. In some embodiments, the second TLR7-selective motif comprises the nucleotide sequence of SEQ ID NO: 37.

In another aspect, provided herein is an immunomodulatory composition that selectively activates TLR7, comprising an RNA ligand and a pharmaceutically acceptable carrier, wherein the RNA ligand comprises: (a) a first TLR7-selective motif comprising the nucleotide sequence of SEQ ID NO: 36 or 37; (b) a second TLR7-selective motif comprising the nucleotide sequence of SEQ ID NO: 36 or 37; (c) a first nucleotide sequence linked to the 5′ end of the first TLR7-selective motif; and (d) a second nucleotide sequence linked to the 3′ end of the first TLR7-selective motif and to the 5′ end of the second TLR7-selective motif.

In some embodiments, which may be combined with any of the preceding aspects or embodiments, the first nucleotide sequence comprises one nucleoside, or at least two nucleosides linked by phosphodiester linkages, wherein the first nucleotide sequence does not comprise a uridine nucleoside, and wherein the first nucleotide sequence is linked to the first TLR7-selective motif by a phosphodiester linkage. In some embodiments, which may be combined with any of the preceding aspects or embodiments, the second nucleotide sequence comprises one nucleoside, or at least two nucleosides linked by phosphodiester linkages, wherein the second nucleotide sequence does not comprise a uridine nucleoside, and wherein the second nucleotide sequence is linked to the first TLR7-selective motif and/or to the second TLR7-selective motif by a phosphodiester linkage. In some embodiments, the first nucleotide sequence and/or the second nucleotide sequence comprise five nucleosides linked by phosphodiester linkages. In some embodiments, the first nucleotide sequence and/or the second nucleotide sequence comprise the nucleotide sequence C-A-G-A-C, or a nucleotide sequence having at least about 85% identity to SEQ ID NO: 67, wherein “-” represents a phosphodiester linkage.

In some embodiments, which may be combined with any of the preceding aspects or embodiments, the RNA ligand comprises the nucleotide sequence of SEQ ID NO: 33, or a nucleotide sequence having at least about 85% identity to SEQ ID NO: 33 and comprising the first TLR7-selective motif of SEQ ID NO: 36 and/or the second TLR7-selective motif of SEQ ID NO: 36. In some embodiments, which may be combined with any of the preceding aspects or embodiments, the RNA ligand comprises the nucleotide sequence of SEQ ID NO: 34, or a nucleotide sequence having at least about 85% identity to SEQ ID NO: 34 and comprising the first TLR7-selective motif of SEQ ID NO: 37 and/or the second TLR7-selective motif of SEQ ID NO: 37.

In another aspect, provided herein is an immunomodulatory composition that selectively activates TLR7, comprising an RNA ligand and a pharmaceutically acceptable carrier, wherein the RNA ligand comprises: (a) a nucleotide sequence of SEQ ID NOs: 33 or 34; (b) a nucleotide sequence having at least about 85% identity to SEQ ID NO: 33 and comprising a first and a second TLR7-selective motif of SEQ ID NO: 36; or (c) a nucleotide sequence having at least about 85% identity to SEQ ID NO: 34 and comprising a first and a second TLR7-selective motif of SEQ ID NO: 37.

In another aspect, provided herein is an immunomodulatory composition that selectively activates TLR7, comprising an RNA ligand and a pharmaceutically acceptable carrier, wherein the RNA ligand comprises: (a) a first TLR7-selective motif and one or more additional TLR7-selective motifs comprising Formula V: U₁—U₂-C₃ (Formula V), wherein U₁ and U₂ are uridine nucleosides and C₃ is a cytidine nucleoside, wherein U₁ has a 2′O-methyl modification (mU), a 2′-fluoro modification (fU), a 2′-amino modification, a 2′-deoxy modification, or a 2′-methoxyethoxy modification, and wherein “-” represents a phosphodiester linkage; and (b) a first nucleotide sequence linked to the 5′ end of the first TLR7-selective motif. In some embodiments, the one or more additional TLR7-selective motifs comprise one, two, three, four, five, or more additional TLR7-selective motifs comprising Formula V. In some embodiments, the one or more additional TLR7-selective motifs comprise five additional TLR7-selective motifs comprising Formula V. In some embodiments, U₁ of the first TLR7-selective motif has a 2′O-methyl modification (mU). In some embodiments, U₁ of at least one of the one or more additional TLR7-selective motifs, or U₁ of all of the one or more additional TLR7-selective motifs, has a 2′O-methyl modification (mU). In some embodiments, U₁ of the first TLR7-selective motif has a 2′-fluoro modification. In some embodiments, U₁ of at least one of the one or more additional TLR7-selective motifs, or U₁ of all of the one or more additional TLR7-selective motifs, has a 2′-fluoro modification. In some embodiments, the first TLR7-selective motif comprises the nucleotide sequence of SEQ ID NO: 40. In some embodiments, at least one of the one or more additional TLR7-selective motifs, or all of the one or more additional TLR7-selective motifs comprise the nucleotide sequence of SEQ ID NO: 40. In some embodiments, the first TLR7-selective motif comprises the nucleotide sequence of SEQ ID NO: 41. In some embodiments, at least one of the one or more additional TLR7-selective motifs, or all of the one or more additional TLR7-selective motifs comprise the nucleotide sequence of SEQ ID NO: 41.

In another aspect, provided herein is an immunomodulatory composition that selectively activates TLR7, comprising an RNA ligand and a pharmaceutically acceptable carrier, wherein the RNA ligand comprises: (a) a first TLR7-selective motif comprising the nucleotide sequence of SEQ ID NO: 40 or 41; (b) one or more additional TLR7-selective motifs, wherein at least one of the one or more additional TLR7-selective motifs, or all of the one or more additional TLR7-selective motifs comprise the nucleotide sequence of SEQ ID NO: 40 or 41; and (c) a first nucleotide sequence linked to the 5′ end of the first TLR7-selective motif. In some embodiments, the one or more additional TLR7-selective motifs comprise one, two, three, four, five, or more additional TLR7-selective motifs comprising the nucleotide sequence of SEQ ID NO: 40 or 41.

In some embodiments, the one or more additional TLR7-selective motifs comprise five additional TLR7-selective motifs comprising the nucleotide sequence of SEQ ID NO: 40 or 41.

In some embodiments, which may be combined with any of the preceding aspects or embodiments, the first nucleotide sequence comprises one nucleoside, or at least two nucleosides linked by phosphodiester linkages, wherein the first nucleotide sequence does not comprise a uridine nucleoside, and wherein the first nucleotide sequence is linked to the first TLR7-selective motif by a phosphodiester linkage. In some embodiments, the first nucleotide sequence comprises two nucleosides linked by phosphodiester linkages. In some embodiments, the first nucleotide sequence comprises two cytidine nucleosides linked by phosphodiester linkages.

In some embodiments, which may be combined with any of the preceding aspects or embodiments, the RNA ligand comprises the nucleotide sequence of SEQ ID NO: 29, or a nucleotide sequence having at least about 85% identity to SEQ ID NO: 29 and comprising the first TLR7-selective motif of SEQ ID NO: 40 and five additional TLR7-selective motifs of SEQ ID NO: 40. In some embodiments, which may be combined with any of the preceding aspects or embodiments, the RNA ligand comprises the nucleotide sequence of SEQ ID NO: 30, or a nucleotide sequence having at least about 85% identity to SEQ ID NO: 30 and comprising the first TLR7-selective motif of SEQ ID NO: 41 and five additional TLR7-selective motifs of SEQ ID NO: 41.

In another aspect, provided herein is an immunomodulatory composition that selectively activates TLR7, comprising an RNA ligand and a pharmaceutically acceptable carrier, wherein the RNA ligand comprises: (a) a nucleotide sequence of SEQ ID NOs: 29 or 30; (b) a nucleotide sequence having at least about 85% identity to SEQ ID NO: 29 and comprising six TLR7-selective motifs of SEQ ID NO: 40; or (c) a nucleotide sequence having at least about 85% identity to SEQ ID NO: 30 and comprising six TLR7-selective motifs of SEQ ID NO: 41.

In another aspect, provided herein is an immunomodulatory composition that selectively activates TLR7, comprising an RNA ligand and a pharmaceutically acceptable carrier, wherein the RNA ligand comprises: (a) a TLR7-selective motif comprising Formula VI: C₁-U₂-U₃*U₄-U₅*C₆ (Formula VI), wherein C₁ and C₆ are cytidine nucleosides, and U₂, U₃, U₄, and U₅ are uridine nucleosides, wherein U₂ has a 2′O-methyl modification (mU), a 2′-fluoro modification (RU), a 2′-amino modification, a 2′-deoxy modification, or a 2′-methoxyethoxy modification, wherein U₄ has a 2′O-methyl modification (mU), a 2′-fluoro modification (fU), a 2′-amino modification, a 2′-deoxy modification, or a 2′-methoxyethoxy modification, and wherein “*” represents a phosphorothioate linkage and “-” represents a phosphodiester linkage; (b) a first nucleotide sequence linked to the 5′ end of the TLR7-selective motif; and (c) a second nucleotide sequence linked to the 3′ end of the TLR7-selective motif. In some embodiments, U₂ of the TLR7-selective motif has a 2′O-methyl modification (mU). In some embodiments, U₄ of the TLR7-selective motif has a 2′O-methyl modification (mU). In some embodiments, U₂ of the TLR7-selective motif has a 2′-fluoro modification (fU). In some embodiments, U₄ of the TLR7-selective motif has a 2′-fluoro modification (RI). In some embodiments, U₂ and U₄ of the TLR7-selective motif have a 2′O-methyl modification (mU). In some embodiments, U₂ and U₄ of the TLR7-selective motif have a 2′-fluoro modification (RI). In some embodiments, U₂ of the TLR7-selective motif has a 2′O-methyl modification (mU), and U₄ of the TLR7-selective motif has a 2′-fluoro modification (RI). In some embodiments, U₂ of the TLR7-selective motif has a 2′-fluoro modification (RI), and U₄ of the TLR7-selective motif has a 2′O-methyl modification (mU). In some embodiments, the TLR7-selective motif comprises the nucleotide sequence of SEQ ID NO: 48. In some embodiments, the TLR7-selective motif comprises the nucleotide sequence of SEQ ID NO: 49. In some embodiments, the TLR7-selective motif comprises the nucleotide sequence of SEQ ID NO: 50. In some embodiments, the TLR7-selective motif comprises the nucleotide sequence of SEQ ID NO: 51.

In another aspect, provided herein is an immunomodulatory composition that selectively activates TLR7, comprising an RNA ligand and a pharmaceutically acceptable carrier, wherein the RNA ligand comprises: (a) a TLR7-selective motif comprising a nucleotide sequence selected from C-mU-U*mU-U*C, C-fU-U*fU-U*C, C-mU-U*fU-U*C, or C-fU-U*mU-U*C;
(b) a first nucleotide sequence linked to the 5′ end of the TLR7-selective motif; and (c) a second nucleotide sequence linked to the 3′ end of the TLR7-selective motif.

In some embodiments, which may be combined with any of the preceding aspects or embodiments, the first nucleotide sequence comprises one nucleoside, or at least two nucleosides linked by phosphodiester linkages, wherein the first nucleotide sequence does not comprise a uridine nucleoside, and wherein the first nucleotide sequence is linked to the TLR7-selective motif by a phosphodiester linkage. In some embodiments, the first nucleotide sequence comprises eight nucleosides linked by phosphodiester linkages. In some embodiments, the first nucleotide sequence comprises the nucleotide sequence C-C-G-A-G-C-C-G, or a nucleotide sequence having at least about 85% identity to SEQ ID NO: 68, wherein “-” represents a phosphodiester linkage, and wherein the first nucleotide sequence is linked to the TLR7-selective motif by a phosphodiester linkage.

In some embodiments, which may be combined with any of the preceding aspects or embodiments, the second nucleotide sequence comprises one nucleoside, or at least two nucleosides linked by phosphodiester linkages, wherein the second nucleotide sequence does not comprise a uridine nucleoside, and wherein the second nucleotide sequence is linked to the TLR7-selective motif by a phosphodiester linkage. In some embodiments, the second nucleotide sequence comprises two nucleosides linked by phosphodiester linkages. In some embodiments, the second nucleotide sequence comprises two cytidine nucleosides linked by phosphodiester linkages.

In some embodiments, which may be combined with any of the preceding aspects or embodiments, the RNA ligand comprises the nucleotide sequence of SEQ ID NO: 17, or a nucleotide sequence having at least about 85% identity to SEQ ID NO: 17 and comprising the TLR7-selective motif of SEQ ID NO: 48. In some embodiments, which may be combined with any of the preceding aspects or embodiments, the RNA ligand comprises the nucleotide sequence of SEQ ID NO: 18, or a nucleotide sequence having at least about 85% identity to SEQ ID NO: 18 and comprising the TLR7-selective motif of SEQ ID NO: 49. In some embodiments, which may be combined with any of the preceding aspects or embodiments, the RNA ligand comprises the nucleotide sequence of SEQ ID NO: 19, or a nucleotide sequence having at least about 85% identity to SEQ ID NO: 19 and comprising the TLR7-selective motif of SEQ ID NO: 50. In some embodiments, which may be combined with any of the preceding aspects or embodiments, the RNA ligand comprises the nucleotide sequence of SEQ ID NO: 20, or a nucleotide sequence having at least about 85% identity to SEQ ID NO: 20 and comprising the TLR7-selective motif of SEQ ID NO: 51.

In another aspect, provided herein is an immunomodulatory composition that selectively activates TLR7, comprising an RNA ligand and a pharmaceutically acceptable carrier, wherein the RNA ligand comprises: (a) a nucleotide sequence selected from SEQ ID NOs: 17, 18, 19, or 20; (b) a nucleotide sequence having at least about 85% identity to SEQ ID NO: 17 and comprising the TLR7-selective motif of SEQ ID NO: 48; (c) a nucleotide sequence having at least about 85% identity to SEQ ID NO: 18 and comprising the TLR7-selective motif of SEQ ID NO: 49; (d) a nucleotide sequence having at least about 85% identity to SEQ ID NO: 19 and comprising the TLR7-selective motif of SEQ ID NO: 50; or (e) a nucleotide sequence having at least about 85% identity to SEQ ID NO: 20 and comprising the TLR7-selective motif of SEQ ID NO: 51.

In some embodiments, which may be combined with any of the preceding aspects or embodiments, the pharmaceutically acceptable carrier is a lipid nanoparticle (LNP). In some embodiments, the LNP comprises a cationic lipid. In some embodiments, the LNP comprises an ionizable lipid.

In some embodiments, which may be combined with any of the preceding aspects or embodiments, the pharmaceutically acceptable carrier is or comprises poly-L-arginine or 1,2-Dioleoyl-3-trimethylammonium propane (DOTAP).

In some embodiments, which may be combined with any of the preceding aspects or embodiments, the immunomodulatory composition selectively activates TLR7 in one or more cells contacted with the immunomodulatory composition. In some embodiments, the one or more cells express TLR7. In some embodiments, the one or more cells comprise peripheral blood mononuclear cells (PBMCs). In some embodiments, the one or more cells comprise one or more plasmacytoid dendritic cells. In some embodiments, the immunomodulatory composition increases TLR7 activity in one or more cells contacted with the immunomodulatory composition, as compared to corresponding one or more cells that are not contacted with the immunomodulatory composition. In some embodiments, the immunomodulatory composition increases secretion of IFN-α by one or more cells contacted with the immunomodulatory composition, as compared to corresponding one or more cells that are not contacted with the immunomodulatory composition. In some embodiments, the increase in TLR7 activity in the one or more cells contacted with the immunomodulatory composition is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition. In some embodiments, the increase in secretion of IFN-α by the one or more cells contacted with the immunomodulatory composition is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition. In some embodiments, the immunomodulatory composition increases TLR8 activity in one or more cells contacted with the immunomodulatory composition by less than about 20%, less than about 15%, less than about 10%, less than about 5%, or less than about 1%, as compared to corresponding one or more cells that are not contacted with the immunomodulatory composition. In some embodiments, the immunomodulatory composition increases secretion of TNF-α by one or more cells contacted with the immunomodulatory composition by less than about 20%, less than about 15%, less than about 10%, less than about 5%, or less than about 1%, as compared to corresponding one or more cells that are not contacted with the immunomodulatory composition. In some embodiments, the RNA ligand is introduced into the one or more cells contacted with the immunomodulatory composition. In some embodiments, the one or more cells contacted with the immunomodulatory composition are further contacted with guanosine or a guanosine derivative, optionally wherein the guanosine derivative is 2′3′ cyclic GMP.

In another aspect, provided herein is an isolated nucleic acid comprising the RNA ligand of any of the immunomodulatory compositions described herein. In some embodiments, the nucleic acid is a DNA, RNA or a DNA/RNA molecule.

In another aspect, provided herein is a vector comprising the RNA ligand of any of the immunomodulatory compositions described herein, or any of the nucleic acids described herein.

In another aspect, provided herein is a pharmaceutical composition comprising any of the immunomodulatory compositions described herein.

In another aspect, provided herein is a host cell comprising the RNA ligand of any of the immunomodulatory compositions described herein, any of the nucleic acids described herein, or any of the vectors described herein.

In another aspect, provided herein is a vaccine composition comprising any of the immunomodulatory compositions described herein or any of the pharmaceutical compositions described herein.

In another aspect, provided herein is a method for selectively activating TLR7 in one or more cells, the method comprising contacting one or more cells with any of the immunomodulatory compositions described herein, or any of the pharmaceutical compositions described herein. In some embodiments, the one or more cells express TLR7. In some embodiments, the one or more cells comprise peripheral blood mononuclear cells (PBMCs). In some embodiments, the one or more cells comprise one or more plasmacytoid dendritic cells. In some embodiments, contacting the one or more cells with the immunomodulatory composition, or the pharmaceutical composition results in an increase in TLR7 activity in the one or more cells, as compared to corresponding one or more cells that are not contacted with the immunomodulatory composition, or the pharmaceutical composition. In some embodiments, contacting the one or more cells with the immunomodulatory composition, or the pharmaceutical composition results in an increase in secretion by the one or more cells of IFN-α, as compared to corresponding one or more cells that are not contacted with the immunomodulatory composition, or the pharmaceutical composition. In some embodiments, the increase in TLR7 activity in the one or more cells is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition, or the pharmaceutical composition. In some embodiments, the increase in secretion by the one or more cells of IFN-α is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition, or the pharmaceutical composition. In some embodiments, contacting the one or more cells with the immunomodulatory composition, or the pharmaceutical composition results in an increase in TLR8 activity in the one or more cells of less than about 20%, less than about 15%, less than about 10%, less than about 5%, or less than about 1%, as compared to corresponding one or more cells that are not contacted with the immunomodulatory composition, or the pharmaceutical composition. In some embodiments, contacting the one or more cells with the immunomodulatory composition, or the pharmaceutical composition results in an increase in secretion by the one or more cells of TNF-α of less than about 20%, less than about 15%, less than about 10%, less than about 5%, or less than about 1%, as compared to corresponding one or more cells that are not contacted with the immunomodulatory composition, or the pharmaceutical composition. In some embodiments, the method further comprises contacting the one or more cells with guanosine or a guanosine derivative, optionally wherein the guanosine derivative is 2′3′ cyclic GMP. In some embodiments, the method further comprises measuring secretion of IFN-α and/or TNF-α by enzyme-linked immunosorbent assay (ELISA), immunoblotting, immunoassays, flow cytometry, electrochemiluminescence-based methods, mass spectrometry, or qPCR; and/or measuring the expression levels of interferon-stimulated genes (ISGs). In some embodiments, contacting the one or more cells with the immunomodulatory composition, or the pharmaceutical composition comprises introducing the RNA ligand into the one or more cells.

In another aspect, provided herein is a method for selectively activating TLR7 in an individual, the method comprising administering to the individual an effective amount of any of the immunomodulatory compositions described herein, any of the pharmaceutical compositions described herein, or any of the vaccine compositions described herein. In some embodiments, administering to the individual an effective amount of the immunomodulatory composition, the pharmaceutical composition, or the vaccine composition results in an increase in TLR7 activity in the individual, as compared to a corresponding individual not administered the immunomodulatory composition, the pharmaceutical composition, or the vaccine composition.

In some embodiments, administering to the individual an effective amount of the immunomodulatory composition, the pharmaceutical composition, or the vaccine composition results in an increase in the individual of the levels of IFN-α, as compared to a corresponding individual not administered the immunomodulatory composition, the pharmaceutical composition, or the vaccine composition. In some embodiments, the increase in TLR7 activity is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition. In some embodiments, the increase of IFN-α levels is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition.

In some embodiments, administering to the individual an effective amount of the immunomodulatory composition, the pharmaceutical composition, or the vaccine composition results in an increase in TLR8 activity in the individual of less than about 20%, less than about 15%, less than about 10%, less than about 5%, or less than about 1%, as compared to a corresponding individual not administered the immunomodulatory composition, the pharmaceutical composition, or the vaccine composition. In some embodiments, administering to the individual an effective amount of the immunomodulatory composition, the pharmaceutical composition, or the vaccine composition results in an increase in levels of TNF-α in the individual of less than about 20%, less than about 15%, less than about 10%, less than about 5%, or less than about 1%, as compared to a corresponding individual not administered the immunomodulatory composition, the pharmaceutical composition, or the vaccine composition.

In another aspect, provided herein is a method for stimulating an immune response in an individual, the method comprising administering to the individual an effective amount of any of the immunomodulatory compositions described herein, any of the pharmaceutical compositions described herein, or any of the vaccine compositions described herein. In some embodiments, administering to the individual an effective amount of the immunomodulatory composition, the pharmaceutical composition, or the vaccine composition results in an increase in TLR7 activity in the individual, as compared to a corresponding individual not administered the immunomodulatory composition, the pharmaceutical composition, or the vaccine composition.

In some embodiments, administering to the individual an effective amount of the immunomodulatory composition, the pharmaceutical composition, or the vaccine composition results in an increase in the individual of the levels of IFN-α, as compared to a corresponding individual not administered the immunomodulatory composition, the pharmaceutical composition, or the vaccine composition. In some embodiments, the increase in TLR7 activity is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition. In some embodiments, the increase of IFN-α levels is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition.

In some embodiments, administering to the individual an effective amount of the immunomodulatory composition, the pharmaceutical composition, or the vaccine composition results in an increase in TLR8 activity in the individual of less than about 20%, less than about 15%, less than about 10%, less than about 5%, or less than about 1%, as compared to a corresponding individual not administered the immunomodulatory composition, the pharmaceutical composition, or the vaccine composition. In some embodiments, administering to the individual an effective amount of the immunomodulatory composition, the pharmaceutical composition, or the vaccine composition results in an increase in levels of TNF-α in the individual of less than about 20%, less than about 15%, less than about 10%, less than about 5%, or less than about 1%, as compared to a corresponding individual not administered the immunomodulatory composition, the pharmaceutical composition, or the vaccine composition.

In another aspect, provided herein is an adjuvant for use in the manufacture of a medicament for activating TLR7 in an individual, wherein the adjuvant comprises any of the immunomodulatory compositions described herein, any of the pharmaceutical compositions described herein, or any of the vaccine compositions described herein.

In another aspect, provided herein is an adjuvant for use in a method for activating TLR7 in an individual, the method comprising administering to the individual an effective amount of any of the immunomodulatory compositions described herein, any of the pharmaceutical compositions described herein, or any of the vaccine compositions described herein.

In another aspect, provided herein is a kit comprising any of the immunomodulatory compositions described herein, any of the nucleic acids described herein, any of the vectors described herein, any of the pharmaceutical compositions described herein, or any of the vaccine compositions described herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIGS. 1A-1B provide crystal structures of Macaca mulatta TLR7 and human TLR8 endosomal leucine-rich repeat domains as activated dimers, with exemplary ligands at Binding Sites #1 and #2. As shown in FIG. 1A, Binding Site #1 of TLR7 binds to guanosines (G), whereas Binding Site #2 binds to a uridine (U) flanked by at least two nucleotides, preferentially pyrimidines. As shown in FIG. 1B, Binding Site #1 of TLR8 binds to uridines (U), whereas Binding Site #2 binds to UG dimers, and can also be occupied by UA or UU dimers, or potentially CG dimers. The asterisks indicate the second subunit of the TLR7 or TLR8 homodimer. See, Miyake et al., International Immunology (2018) 30(2):43-51. See, also: Zhang et al., Immunity (2016) 45:737; and Tanji et al., Nature Structural & Molecular Biology (2015) 22:109-115.

FIG. 2 depicts the reaction catalyzed by an RNase enzyme. RNases catalyze nucleophilic attack at the 3′phosphorus (top), leading to the release of the 3′ nucleotide with a 5′OH. The 3′ end is characterized by a 2′3′cyclophosphate.

FIG. 3 shows the chemical structures of 2′-O-methyl, 2′-fluoro, 2′-deoxy, 2′-amino, 2′-methoxyethoxy, and phosphorothioate (pto) modifications. The “U” or “N” at the 1′ position is representative of any possible nucleobase.

FIG. 4 shows the secondary structure adopted by ORN7023 (derived from WO2010/105819), which has the nucleotide sequence C-G-G-C-U-C-G-G-C-A-G-A-A-G-C-C-G-G-G-C-C(SEQ ID NO: 2; “-” represents a phosphodiester linkage). ORN7023 forms a short canonical RNA hairpin, including a G:U wobble base pair that is indicated by the box. The secondary structure of ORN7023 was obtained using RNAfold (see, e.g., Gruber et al., Nucleic Acids Research (2008) 36:2s:W70-W74; Lorenz et al., Algorithms for Molecular Biology (2011) 6:1:26; and rna.tbi.univie.ac.at/cgi-bin/RNAWebSuite/RNAfold.cgi).

FIG. 5 depicts Binding Site #2 of TLR7 within a TLR7 dimer. A U₃-trimer is bound within Binding Site #2. U₁, U₂ and U₃ represent nucleotides in the 5′ to 3′ orientation. The solid triangles label the 2′OH moieties of the three nucleotides (U₁, U₂ and U₃). Each TLR7 subunit is indicated by either light blue or pink shading. The asterisk indicates the second subunit of the TLR7 homodimer. See, Zhang et al., Cell Reports (2018) 25:3371-3381.

FIG. 6 depicts nucleic acid receptor expression and cytokine secretion by peripheral blood mononuclear cells (PBMCs). The main cell types in PBMCs are shown across the top of FIG. 6 (cDC=conventional dendritic cell; pDC=plasmacytoid dendritic cell; NK=natural killer). The cytokine-inducing nucleic acid receptors that are expressed by pDCs and monocytes are shown on the bottom of FIG. 6.

FIG. 7 shows the results of experiments that measured the level of TNF-α or IFN-α secretion by PBMCs in response to the RNA ligands described in Example 1. Human PBMCs were stimulated with poly-L-arginine (p-L-arginine)-complexed RNA ligands (indicated on the x-axis), either alone or in combination with guanosine (as indicated by the legend; “noG” indicates that the PBMCs were stimulated with the p-L-arginine-complexed RNA ligands alone, and “G” indicates that the PBMCs were co-stimulated with the p-L-arginine-complexed RNA ligands and guanosine). The levels of TNF-α and IFN-α secretion were measured 16 hours after p-L-arginine delivery of the RNA ligands, as shown on the y-axes (pg/ml). “Media” refers to the negative control (no stimulation of the PBMCs with either p-L-arginine-complexed RNA ligands or guanosine). “R848” is the positive control and refers to PBMCs stimulated with R848, a small molecule TLR7/8 ligand, also called resiquimod, at a concentration of 1 pg/ml.

FIG. 8 shows the results of experiments that measured the level of TNF-α and IFN-α secretion by PBMCs or pDC-depleted PBMCs in response to certain RNA ligands described in Example 1. Human PBMCs or pDC-depleted PBMCs were stimulated with p-L-arginine-complexed RNA ligands, as indicated on the x-axis. The levels of TNF-α and IFN-α secretion were measured 16 hours after p-L-arginine delivery of the RNA ligands, as shown on the y-axes (pg/ml). “Media” refers to the negative control (no stimulation of the PBMCs with p-L-arginine-complexed RNA ligands). The 9.2S RNA sample is human PBMCs or pDC-depleted PBMCs stimulated with p-L-arginine-complexed with the TLR7/8 activating 9.2S RNA. The 9.2S RNA is a positive control with the nucleotide sequence A-G-C-U-U-A-A-C-C-U-G-U-C-C-U-U-C-A-A (SEQ ID NO: 56; “-” represents a phosphodiester linkage). “IVT4” refers to human PBMCs or pDC-depleted PBMCs stimulated with a Lipofectamine-complexed control having the nucleotide sequence G-G-G-A-C-G-C-U-G-A-C-C-C-A-G-A-A-G-A-U-C-A-C-U-A-G-A-A-A-U-A-G-U-A-G-A-U-C-U-U-C-U-G-G-G-U-C-A-G-C-G-U-C-C-C (SEQ ID NO: 62; “-” represents a phosphodiester linkage). The IVT4 control was generated by in vitro transcription and is chloroquine resistant and activates RIG-I, but not TLR7/8.

FIG. 9 shows the results of experiments that characterized the size of the nanoparticles formed by the indicated RNA ligands complexed with p-L-arginine (pLA).

FIGS. 10A-10B show the results of experiments that measured the level of TNF-α or IFN-α secretion by PBMCs in response to the indicated RNA ligands described in Example 1, delivered with DOTAP. Human PBMCs were stimulated with DOTAP-complexed RNA ligands (indicated on the x-axes) at a concentration of 0.6 μg/ml (FIG. 10A) or 2 μg/ml (FIG. 10B). The levels of TNF-α and IFN-α secretion were measured 16 hours after DOTAP delivery of the RNA ligands, as shown on the y-axes (pg/ml). The pCA, pCA-2, and pCA-3 samples are negative control RNA oligonucleotides that do not induce a cytokine response. pCA, pCA-2, and pCA-3 comprise the nucleotide sequence C-A-C-A-C-A-C-A-C-A-C-A-C-A-C-A-C-A-C-A (SEQ ID NO: 70), wherein “-” indicates a phosphodiester linkage. The pU2G sample is a positive control RNA oligonucleotide that induces both TLR7 and TLR8. pU2G comprises the nucleotide sequence G-G-U-U-G-U-U-G-U-U-G-U-U-G-U-U-G-U-U-G (SEQ ID NO: 71), wherein “-” indicates a phosphodiester linkage.

FIG. 11 shows the results of experiments that measured the level of TNF-α or IFN-α secretion by PBMCs in response to the indicated RNA ligands described in Example 1, delivered without a carrier (i.e., free RNA ligands). Human PBMCs were stimulated with free RNA ligands (indicated on the x-axis) alone. The levels of TNF-α and IFN-α secretion were measured 16 hours after delivery, as shown on the y-axis (pg/ml). The pCA and pCA-2 samples are negative control RNA oligonucleotides that do not induce a cytokine response (SEQ ID NO: 70). The pU2G_pto sample is a positive control RNA oligonucleotide that induces both TLR7 and TLR8. pU2G_pto comprises the nucleotide sequence G*G*U*U*G*U*U*G*U*U*G*U*U*G*U*U*G*U*U*G (SEQ ID NO: 72), wherein indicates a phosphorothioate linkage.

FIG. 12 shows the results of an experiment in which ORNs 7005-7012 were delivered to human PBMCs with p-L-arginine alone (“no 2′3′ cGMP”), or complexed with p-L-arginine and 2′3′ cyclic GMP (“+2′3′ cGMP”). The final concentration of 2′3′ cyclic GMP was 5 μg/ml, and the ORNs were all used at a concentration of 0.6 μg/ml. Cytokine secretion was measured as in FIG. 7. The PBMCs were obtained from 6 human donors. The results shown represent two independent experiments.

FIG. 13 shows the results of an experiment performed as in FIG. 8, except that in all conditions, the RNA ligands were complexed together with 2′3′ cyclic GMP (“2′3′ cGMP”). The final concentration of 2′3′ cyclic GMP was 5 μg/ml, and the RNA ligands (ORNs 7005-7012) were used at a concentration of 0.6 μg/ml. Human PBMCs were obtained from 3 donors. The results shown represent one experiment.

FIG. 14 shows the results of an experiment as performed in FIG. 8, but using human PBMCs obtained from three new donors.

FIGS. 15A-15B show the results of an experiment that assessed the effect of RNA modifications at the 2′ position on RNAse cleavage activity. FIG. 15A shows the unmodified sequences corresponding to ORN7009 and ORN7013, with potential RNAse T2 cleavage sites indicated with arrows. The size of the arrow indicates the strength of the consensus sequence for RNase T2 cleavage, and therefore the likelihood of a cleavage event. The cleavage sites were based on the literature (Ostendorf et al., (2020) 52(4):591-605). FIG. 15B shows TBE-Urea PAGE gels loaded with 6 μl of each reaction containing RNase T2 and unmodified RNA ligands (ORN7009 and ORN7013), or 3 μl of each reaction with RNase T2 and modified RNA ligands (ORN7014-7017 and ORN7010-7012). For the “No RNAse” control, 3 μl of each RNA ligand were incubated in the same reaction conditions, minus RNase T2 for 12 minutes. The top gel in FIG. 15B shows RNA ligands ORN7009-7012 incubated with 0.2 μg/ml RNase T2 for 3, 6, 9 or 12 minutes. The bottom gel in FIG. 15B shows RNA ligands ORN7013-7017 incubated with 0.5 μg/ml RNase T2 for 3, 6, 9 or 12 minutes.

FIGS. 16A-16B show the IFN-α data plotted in FIG. 12 with individual data points indicating unique human PBMC donors across two separate experiments. FIG. 16A and FIG. 16B provide context to any variability seen in FIG. 12 by illustrating the reproducibility across donors and experiments.

### DETAILED DESCRIPTION

### Definitions

As used herein and in the appended claims, the singular forms “a,” “or,” and “the” include plural referents unless the context clearly dictates otherwise. As used herein, “about” refers to the usual error range for the respective value or parameter readily known to the skilled person in this technical field. Reference to “about” a value or parameter herein includes (and describes) embodiments that are directed to that value or parameter per se. It is understood that aspects and embodiments of the present disclosure described herein include “comprising,” “consisting,” and “consisting essentially of” aspects and embodiments.

As used herein, the term “immunomodulatory” refers to the ability of an agent, such as a composition or an RNA ligand of the disclosure, to modulate the immune system of a host (e.g., an individual, e.g., as described herein). Such modulation may include increasing one or more immune responses, such as, but not limited to, increased antibody production (e.g., antigen-specific antibody production); activation or proliferation of immune system cells, such as T cells, B cells, natural killer cells, dendritic cells, macrophages, and the like; increased production or release (e.g., secretion) of immunostimulatory cytokines; or direct or indirect enhancement of adaptive, innate, cellular or humoral immune responses. Such modulation may also include decreasing one or more immune responses, such as, but not limited to, reduction in antigen-specific antibody production; activation of lymphocyte or other cell populations that have immunosuppressive activities; increased synthesis of cytokines that have suppressive effects toward certain cellular functions; or directly or indirectly decreasing certain adaptive, innate, cellular or humoral immune responses.

As used herein, the terms “selective,” “selectivity,” and “selectively” with respect to TLR7 activity refer to the ability of an agent, such as an immunomodulatory composition or an RNA ligand of the disclosure, to increase the activity of TLR7, e.g., in one or more cells, to a greater degree than the activity of another molecule, such as another Toll-like receptor, e.g., TLR8. Thus, a TLR7-selective RNA ligand or immunomodulatory composition of the disclosure may increase the activity of TLR7, and also may increase the activity of another molecule, such as another Toll-like receptor, e.g., TLR8, however, the increase in the activity of TLR7 will be to a greater degree than the increase in activity of the other molecule (e.g., TLR8).

As used herein, an “RNA ligand” refers to a nucleotide polymer of any length. The nucleotides can be ribonucleotides, modified ribonucleotides or bases, and/or their analogs, or any substrate that can be incorporated into a polymer by an RNA polymerase or by a synthetic reaction. A polynucleotide may comprise modified nucleotides. If present, modification to the nucleotide structure may be imparted before or after assembly of the polymer. The sequence of nucleotides may be interrupted by non-nucleotide components.

As used herein, a “pharmaceutically acceptable carrier” refers to an ingredient in a composition, other than an active ingredient, which is nontoxic to an individual.

As used herein, “percent (%) identity” with respect to a specified subject sequence, or a specified portion thereof, is defined as the percentage of nucleotides in an identified sequence identical with the nucleotides in the subject sequence (or specified portion thereof), after aligning the sequences and introducing gaps, if necessary to achieve the maximum percent sequence identity.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “vector,” as used herein, refers to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked.

The term “host cell,” refers to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual is a human.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication, delaying the progression of the disease, and/or prolonging survival. An effective amount of drug, compound, or composition is an amount sufficient to accomplish prophylactic or therapeutic treatment either directly or indirectly. As is understood in the clinical context, an effective amount of a drug, compound, or composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

### Overview

Certain aspects of the present disclosure relate to immunomodulatory compositions comprising RNA ligands that selectively activate TLR7.

### Difficulties in the Development of TLR7-Selective Ligands

While multiple TLR8-selective ligands have been described, TLR7-selective RNA ligands are very rare. Some approaches have been attempted to generate TLR7-selective ligands. For example, secondary structures, such as hairpins or G:U wobble base pairs within short double stranded RNA (dsRNA) sequences have been described as selectively activating TLR7. However, the mechanism leading to the TLR7-selectivity remains unclear and cannot be explained by TLR7/8 recognition motifs alone. See, e.g., WO2010/105819. It is possible, however, that such secondary structures favor RNase degradation patterns that are unfavorable for TLR8 activation. However, Applicants recognized that ligands that rely solely on hairpin structures and/or G:U wobble base pairs would require such structures as fixed secondary structures and could also have variable activity with different delivery methods.

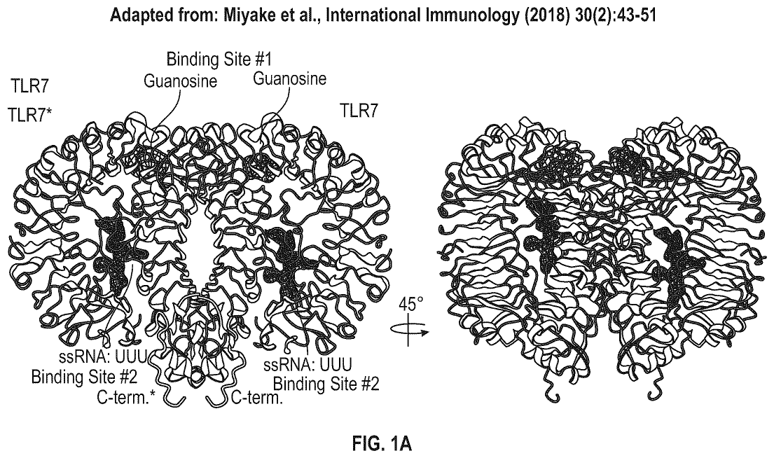

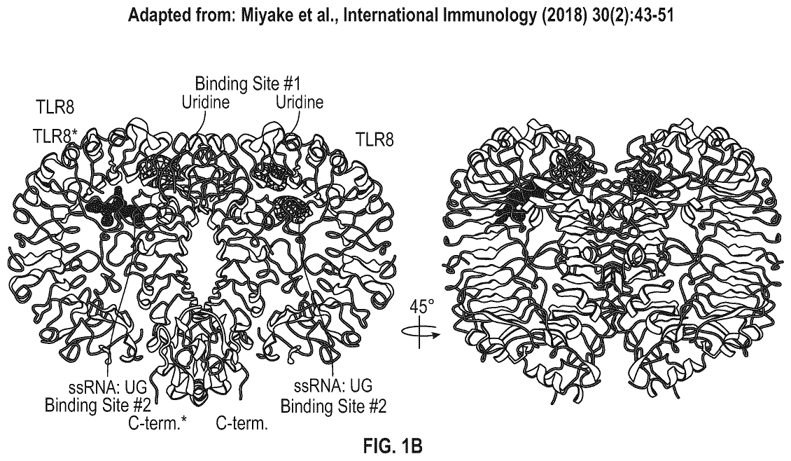

Applicants recognized that a possible reason for the lack of TLR7-selective ligands could be the specificity of the two nucleotide binding sites in TLR7 and TLR8, termed Binding Site #1 and Binding Site #2 (FIGS. 1A-1B). Without wishing to be bound by theory, Applicants considered this property when designing the TLR7-selective ligands of this disclosure.

Binding Site #1 is of higher importance and binds monomeric nucleosides or potentially 3′2′cyclophospo-nucleosides, which are typical products of RNase degradation. As depicted in FIGS. 1A-1B, TLR7 Binding Site #1 binds to guanosines (FIG. 1A), whereas TLR8 Binding Site #1 binds to uridines (FIG. 1B). Binding Site #1 is also bound by a range of synthetic small molecule activators such as resiquimod, gardiquimod or imiquimod. Due to their high affinities, binding of synthetic small molecules to Binding Site #1 alone is sufficient for activation. However, full activation by RNA ligands also requires binding at Binding Site #2.

Binding Site #2 is both qualitatively and spatially distinct in TLR7 and TLR8 (FIGS. 1A-1B). TLR7 Binding Site #2 appears to strictly require a uridine flanked by two nucleotides, preferentially pyrimidines that may be embedded in a longer RNA oligoribonucleotide (FIG. 1A). On the other hand, TLR8 binds UG dimers at Binding Site #2, but is also more promiscuous and can be occupied by UA or UU dimers, or potentially CG dimers (FIG. 1B). The 3′end is defined by a 3′2′ cyclic phosphate, while the 5′ end is less well defined and may be part of a trimer or even a longer oligo.

Therefore, the difficulty in generating TLR7-selective ligands could be due, at least in part, to the fact that TLR7 appears to strictly require both guanosines (for Binding Site #1) and uridines (for Binding Site #2) for activation, while TLR8 only requires uridines. Therefore, any U- or UA-restricted sequence may activate TLR8 but not TLR7, while the requirement of uridines for TLR7 Binding Site #2 means that sequences activating TLR7 will harbor the potential to generate TLR8 ligands if relying only on sequence for selectivity.

### Development of TLR7-Selective Ligands Using RNA Modifications

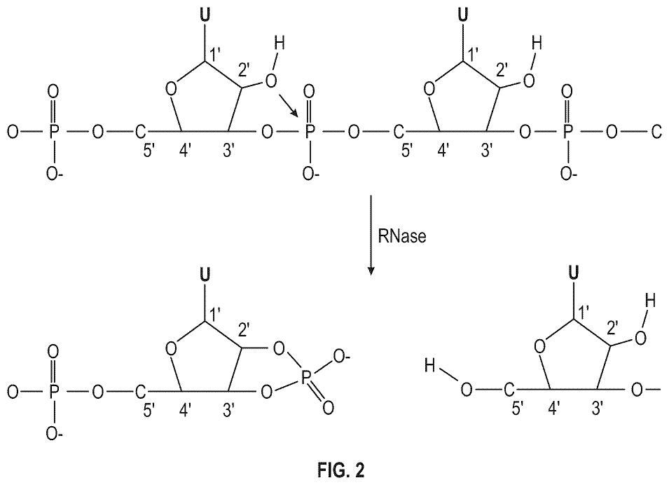

RNases are enzymes that cleave RNA and can generate RNA degradation products that can act as ligands for TLR7 and/or TLR8 activation. As depicted in FIG. 2, RNases cleave RNA molecules 3′ of a nucleotide by a nucleophilic attack of the 2′ oxygen at the 3′ phosphorus, resulting in the release of the 3′ nucleotide with a 5′OH and the generation of a 2′-3′ cyclophosphate, which is a widely used marker of RNase activity.

Certain RNA modifications interfere with the activity of RNases. For example, phosphorothioate modifications introduce chirality to the phosphate of the backbone, and have been shown to slow down RNA degradation and enhance TLR activation. In addition, modification of the 2′ position on the ribose may deprive the RNA backbone of the 2′OH required for RNase activity and/or block binding of any nucleases.

Applicants recognized that introduction of certain RNA modifications at specific sites within oligoribonucleotide sequences could interfere with RNase activity and prevent the formation of RNA degradation products that activate TLR8, while allowing the release of ligands that activate TLR7.

Accordingly, certain aspects of the present disclosure are based, at least in part, on the discovery of RNA ligands with specific RNA modifications at specific sites (see, e.g., Example 1) that enhance TLR7-selectivity and result in high levels of TLR7 activity (see, e.g., Example 2), thereby overcoming the difficulties with generating TLR7-selective ligands.

### Compositions of the Disclosure

In some aspects, provided herein are immunomodulatory compositions that selectively activate TLR7. In some embodiments, the immunomodulatory compositions of the disclosure comprise an RNA ligand, e.g., an RNA ligand described herein. In some embodiments, the RNA ligand comprises one or more TLR7-selective motifs, such as a TLR7-selective motif described herein, comprising one or more RNA modifications, e.g. one or more of the RNA modifications described herein. In some embodiments, the immunomodulatory compositions of the disclosure further comprise a carrier, such as a pharmaceutically acceptable carrier, e.g., as described herein.

### RNA Ligands

In some aspects, provided herein are RNA ligands comprising one or more TLR7-selective motifs, wherein the TLR7-selective motifs comprise one or more of the RNA modifications described herein.

RNA Ligands Comprising TLR7-Selective Motif of Formula I

In one aspect, provided herein are RNA ligands comprising a TLR7-selective motif comprising Formula I:

N-/*U (Formula I),

wherein N is any nucleoside comprising a modification at the 2′ position of the ribose, U is a uridine nucleoside, and “-/*” represents a phosphodiester linkage or a phosphorothioate linkage.

In some embodiments, N comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′ deoxy modification, or a 2′methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′ deoxy modification. In some embodiments, the modification is a 2′methoxyethoxy modification.

In some embodiments, the RNA ligand further comprises additional nucleotide sequences that flank the 5′ end of the TLR7-selective motif, the 3′ end of the TLR7-selective motif, or both. In some embodiments, the additional nucleotide sequences do not comprise a uridine nucleoside.

In some embodiments, the RNA ligand further comprises a first nucleotide sequence that is linked to the 5′ end of the TLR7-selective motif. In some embodiments, the first nucleotide sequence comprises at least one nucleoside. In some embodiments, the first nucleotide sequence comprises at least two nucleosides. In some embodiments, the nucleosides of the first nucleotide sequence are linked by phosphodiester linkages or phosphorothioate linkages. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence is linked to the 5′ end of the TLR7-selective motif by a phosphodiester linkage or a phosphorothioate linkage. In some embodiments, the first nucleotide sequence does not comprise a uridine nucleoside.

In some embodiments, the RNA ligand further comprises a second nucleotide sequence that is linked to the 3′ end of the TLR7-selective motif. In some embodiments, the second nucleotide sequence comprises at least one nucleoside. In some embodiments, the second nucleotide sequence comprises at least two nucleosides. In some embodiments, the nucleosides of the second nucleotide sequence are linked by phosphodiester linkages or phosphorothioate linkages. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence is linked to the 3′ end of the TLR7-selective motif by a phosphodiester linkage or a phosphorothioate linkage. In some embodiments, the second nucleotide sequence does not comprise a uridine nucleoside.

In some embodiments, the sequence of the first nucleotide sequence is identical to the sequence of the second nucleotide sequence. In some embodiments, the sequence of the first nucleotide sequence is different from the sequence of the second nucleotide sequence.

In some embodiments, the RNA ligand comprises two or more TLR7-selective motifs comprising Formula I. In some embodiments, the RNA ligand comprises two or more TLR7-selective motifs comprising Formula I and at least two additional nucleotide sequences, wherein the additional nucleotide sequences comprises at least one nucleoside. The TLR7-selective motifs and the at least two additional nucleotide sequences may be arranged in any order on the RNA ligand. In one non-limiting example, an RNA ligand may comprise, in the 5′ to 3′ direction, a first nucleotide sequence, a TLR7-selective motif of Formula I, one or more additional TLR7-selective motifs of Formula I (e.g., in tandem), a second nucleotide sequence, one or more additional TLR7-selective motifs of Formula I (e.g., in tandem), and a third nucleotide sequence. In some embodiments, the first, second, and/or third nucleotide sequences comprise at least one nucleoside. In some embodiments, the first, second, and/or third nucleotide sequences comprise at least two nucleosides. In some embodiments, the nucleosides of the first, second, and/or third nucleotide sequences are linked by phosphodiester linkages or phosphorothioate linkages. In some embodiments, the first, second, and/or third nucleotide sequences comprise any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first, second, and/or third nucleotide sequences are linked to adjacent TLR7-selective motifs by phosphodiester linkages or phosphorothioate linkages. In some embodiments, the first, second, and/or third nucleotide sequences do not comprise a uridine nucleoside. Those skilled in the art can arrive at other RNA ligands that comprise two or more TLR7-selective motifs comprising Formula I and at least two additional nucleotide sequences (e.g., as described above), wherein the TLR7-selective motifs and the at least two additional nucleotide sequences are arranged in any order on the RNA ligand.

RNA Ligands Comprising TLR7-Selective Motif of Formula II

In one aspect, provided herein are RNA ligands comprising a TLR7-selective motif comprising Formula II:

C₁*U₂-U₃*U₄-U₅*C₆ (Formula II),

wherein C₁ and C₆ are cytidine nucleosides, and U₂, U₃, U₄, and U₅ are uridine nucleosides, wherein “*” represents a phosphorothioate linkage and “-” represents a phosphodiester linkage.

In some embodiments, U₂ comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification.

In some embodiments, U₄ comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification.

In some embodiments, the ribose modification of U₂ is a 2′O-methyl modification, and the ribose modification of U₄ is a 2′O-methyl modification. In some embodiments, the ribose modification of U₂ is a 2′O-methyl modification, and the ribose modification of U₄ is a 2′-fluoro modification. In some embodiments, the ribose modification of U₂ is a 2′O-methyl modification, and the ribose modification of U₄ is a 2′-amino modification. In some embodiments, the ribose modification of U₂ is a 2′O-methyl modification, and the ribose modification of U₄ is a 2′-deoxy modification. In some embodiments, the ribose modification of U₂ is a 2′O-methyl modification, and the ribose modification of U₄ is a 2′-methoxyethoxy modification. In some embodiments, the ribose modification of U₂ is a 2′-fluoro modification, and the ribose modification of U₄ is a 2′O-methyl modification. In some embodiments, the ribose modification of U₂ is a 2′-fluoro modification, and the ribose modification of U₄ is a 2′-fluoro modification. In some embodiments, the ribose modification of U₂ is a 2′-fluoro modification, and the ribose modification of U₄ is a 2′-amino modification. In some embodiments, the ribose modification of U₂ is a 2′-fluoro modification, and the ribose modification of U₄ is a 2′-deoxy modification. In some embodiments, the ribose modification of U₂ is a 2′-fluoro modification, and the ribose modification of U₄ is a 2′-methoxyethoxy modification. In some embodiments, the ribose modification of U₂ is a 2′-amino modification, and the ribose modification of U₄ is a 2′O-methyl modification. In some embodiments, the ribose modification of U₂ is a 2′-amino modification, and the ribose modification of U₄ is a 2′-fluoro modification. In some embodiments, the ribose modification of U₂ is a 2′-amino modification, and the ribose modification of U₄ is a 2′-amino modification. In some embodiments, the ribose modification of U₂ is a 2′-amino modification, and the ribose modification of U₄ is a 2′-deoxy modification. In some embodiments, the ribose modification of U₂ is a 2′-amino modification, and the ribose modification of U₄ is a 2′-methoxyethoxy modification. In some embodiments, the ribose modification of U₂ is a 2′-deoxy modification, and the ribose modification of U₄ is a 2′O-methyl modification. In some embodiments, the ribose modification of U₂ is a 2′-deoxy modification, and the ribose modification of U₄ is a 2′-fluoro modification. In some embodiments, the ribose modification of U₂ is a 2′-deoxy modification, and the ribose modification of U₄ is a 2′-amino modification. In some embodiments, the ribose modification of U₂ is a 2′-deoxy modification, and the ribose modification of U₄ is a 2′-deoxy modification. In some embodiments, the ribose modification of U₂ is a 2′-deoxy modification, and the ribose modification of U₄ is a 2′-methoxyethoxy modification. In some embodiments, the ribose modification of U₂ is a 2′-methoxyethoxy modification, and the ribose modification of U₄ is a 2′O-methyl modification. In some embodiments, the ribose modification of U₂ is a 2′-methoxyethoxy modification, and the ribose modification of U₄ is a 2′-fluoro modification. In some embodiments, the ribose modification of U₂ is a 2′-methoxyethoxy modification, and the ribose modification of U₄ is a 2′-amino modification. In some embodiments, the ribose modification of U₂ is a 2′-methoxyethoxy modification, and the ribose modification of U₄ is a 2′-deoxy modification. In some embodiments, the ribose modification of U₂ is a 2′-methoxyethoxy modification, and the ribose modification of U₄ is a 2′-methoxyethoxy modification.

In some embodiments, the TLR7-selective motif comprises the nucleotide sequence of SEQ ID NO: 44. In some embodiments, the TLR7-selective motif comprises the nucleotide sequence of SEQ ID NO: 45. In some embodiments, the TLR7-selective motif comprises the nucleotide sequence of SEQ ID NO: 46. In some embodiments, the TLR7-selective motif comprises the nucleotide sequence of SEQ ID NO: 47.

In some embodiments, the RNA ligand further comprises additional nucleotide sequences that flank the 5′ end of the TLR7-selective motif, the 3′ end of the TLR7-selective motif, or both. In some embodiments, the additional nucleotide sequences do not comprise a uridine nucleoside.

In some embodiments, the RNA ligand comprises a first nucleotide sequence that is linked to the 5′ end of the TLR7-selective motif. In some embodiments, the first nucleotide sequence comprises at least one nucleoside. In some embodiments, the first nucleotide sequence comprises at least two nucleosides. In some embodiments, the nucleosides of the first nucleotide sequence are linked by phosphodiester linkages or phosphorothioate linkages. In some embodiments, the nucleosides of the first nucleotide sequence are linked by phosphorothioate linkages. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises 8 nucleosides. In some embodiments, the first nucleotide sequence comprises the nucleotide sequence of SEQ ID NO: 58, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 58. In some embodiments, the first nucleotide sequence is linked to the 5′ end of the TLR7-selective motif by a phosphodiester linkage or a phosphorothioate linkage. In some embodiments, the first nucleotide sequence is linked to the 5′ end of the TLR7-selective motif by a phosphorothioate linkage.

In some embodiments, the RNA ligand comprises a second nucleotide sequence that is linked to the 3′ end of the TLR7-selective motif. In some embodiments, the second nucleotide sequence comprises at least one nucleoside. In some embodiments, the second nucleotide sequence comprises at least two nucleosides. In some embodiments, the nucleosides of the second nucleotide sequence are linked by phosphodiester linkages or phosphorothioate linkages.

In some embodiments, the nucleosides of the second nucleotide sequence are linked by phosphorothioate linkages. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 2 nucleosides. In some embodiments, the second nucleotide sequence comprises the nucleotide sequence of SEQ ID NO: 59, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 59. In some embodiments, the second nucleotide sequence is linked to the 3′ end of the TLR7-selective motif by a phosphodiester linkage or a phosphorothioate linkage. In some embodiments, the second nucleotide sequence is linked to the 3′ end of the TLR7-selective motif by a phosphorothioate linkage.

In another aspect, provided herein is an RNA ligand comprising a TLR7-selective motif comprising a nucleotide sequence of C*mU-U*mU-U*C, C*fU-U*fU-U*C, C*mU-U*fU-U*C, and C*fU-U*mU-U*C. In some embodiments, the RNA ligand comprises a TLR7-selective motif comprising a nucleotide sequence of SEQ ID NO: 44. In some embodiments, the RNA ligand comprises a TLR7-selective motif comprising a nucleotide sequence of SEQ ID NO: 45. In some embodiments, the RNA ligand comprises a TLR7-selective motif comprising a nucleotide sequence of SEQ ID NO: 46. In some embodiments, the RNA ligand comprises a TLR7-selective motif comprising a nucleotide sequence of SEQ ID NO: 47. In some embodiments, the RNA ligand further comprises additional nucleotide sequences that flank the 5′ end of the TLR7-selective motif, the 3′ end of the TLR7-selective motif, or both. In some embodiments, the additional nucleotide sequences do not comprise a uridine nucleoside. In some embodiments, the RNA ligand further comprises a first nucleotide sequence that is linked to the 5′ end of the TLR7-selective motif. In some embodiments, the RNA ligand further comprises a second nucleotide sequence that is linked to the 3′ end of the TLR7-selective motif. In some embodiments, the first nucleotide sequence comprises at least one nucleoside. In some embodiments, the first nucleotide sequence comprises at least two nucleosides. In some embodiments, the nucleosides of the first nucleotide sequence are linked by phosphodiester linkages or phosphorothioate linkages. In some embodiments, the nucleosides of the first nucleotide sequence are linked by phosphorothioate linkages. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises 8 nucleosides. In some embodiments, the first nucleotide sequence comprises the nucleotide sequence of SEQ ID NO: 58, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 58. In some embodiments, the first nucleotide sequence is linked to the 5′ end of the TLR7-selective motif by a phosphodiester linkage or a phosphorothioate linkage. In some embodiments, the first nucleotide sequence is linked to the 5′ end of the TLR7-selective motif by a phosphorothioate linkage. In some embodiments, the second nucleotide sequence comprises at least one nucleoside. In some embodiments, the second nucleotide sequence comprises at least two nucleosides. In some embodiments, the nucleosides of the second nucleotide sequence are linked by phosphodiester linkages or phosphorothioate linkages. In some embodiments, the nucleosides of the second nucleotide sequence are linked by phosphorothioate linkages. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 2 nucleosides. In some embodiments, the second nucleotide sequence comprises the nucleotide sequence of SEQ ID NO: 59, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 59. In some embodiments, the second nucleotide sequence is linked to the 3′ end of the TLR7-selective motif by a phosphodiester linkage or a phosphorothioate linkage. In some embodiments, the second nucleotide sequence is linked to the 3′ end of the TLR7-selective motif by a phosphorothioate linkage.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 12 and comprising the TLR7-selective motif of SEQ ID NO: 44. In some embodiments, the RNA ligand comprises the nucleotide sequence of SEQ ID NO: 12.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 13 and comprising the TLR7-selective motif of SEQ ID NO: 45. In some embodiments, the RNA ligand comprises the nucleotide sequence of SEQ ID NO: 13.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 14 and comprising the TLR7-selective motif of SEQ ID NO: 46. In some embodiments, the RNA ligand comprises the nucleotide sequence of SEQ ID NO: 14.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 15 and comprising the TLR7-selective motif of SEQ ID NO: 47. In some embodiments, the RNA ligand comprises the nucleotide sequence of SEQ ID NO: 15.

RNA Ligands Comprising TLR7-Selective Motif of Formula III

In one aspect, provided herein are RNA ligands comprising a TLR7-selective motif comprising Formula III:

C₁—U₂-C₃ (Formula III),

wherein C₁ and C₃ are cytidine nucleosides and U₂ is a uridine nucleoside, and wherein “-” represents a phosphodiester linkage.

In some embodiments, C₁ comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification.

In some embodiments, the TLR7-selective motif comprises the nucleotide sequence of SEQ ID NO: 42. In some embodiments, the TLR7-selective motif comprises the nucleotide sequence of SEQ ID NO: 43.

In some embodiments, the RNA ligand further comprises additional nucleotide sequences that flank the 5′ end of the TLR7-selective motif, the 3′ end of the TLR7-selective motif, or both. In some embodiments, the additional nucleotide sequences do not comprise a uridine nucleoside.

In some embodiments, the RNA ligand further comprises a first nucleotide sequence linked to the 5′ end of the TLR7-selective motif. In some embodiments, the first nucleotide sequence does not comprise a uridine nucleoside. In some embodiments, the first nucleotide sequence is linked to the TLR7-selective motif by a phosphodiester linkage. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises three nucleosides. In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises the nucleotide sequence C-G-G, wherein “-” represents a phosphodiester linkage.

In some embodiments, the RNA ligand further comprises a second nucleotide sequence linked to the 3′ end of the TLR7-selective motif. In some embodiments, the second nucleotide sequence does not comprise a uridine nucleoside. In some embodiments, the second nucleotide sequence comprises a nucleotide sequence that is capable of hybridizing to all or a part of the first nucleotide sequence, and/or all or a part of the TLR7-selective motif. In some embodiments, the second nucleotide sequence comprises a nucleotide sequence that is capable of hybridizing to all or a part of the first nucleotide sequence, and/or all or a part of the TLR7-selective motif, wherein the hybridized sequences form a G:U wobble base pair. In some embodiments, the second nucleotide sequence comprises the nucleotide sequence G-G-G. In some embodiments, the nucleotide sequence G-G-G is capable of base pairing with the TLR7-selective motif. In some embodiments, the nucleotide sequence G-G-G hybridizes to the TLR7-selective motif and forms a G:U wobble base pair. In some embodiments, the second nucleotide sequence is linked to the TLR7-selective motif by a phosphodiester linkage. In some embodiments, the second nucleotide sequence comprises at least three nucleosides. In some embodiments, the second nucleotide sequence comprises any of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 15 nucleosides. In some embodiments, the nucleosides within the second nucleotide sequence are linked by phosphodiester linkages or phosphorothioate linkages. In some embodiments, the nucleosides within the second nucleotide sequence are linked by phosphodiester linkages. In some embodiments, the second nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 61 and comprising the nucleotide sequence of SEQ ID NO: 69, wherein the nucleotide sequence of SEQ ID NO: 69 is capable of base pairing with the TLR7-selective motif. In some embodiments, the second nucleotide sequence comprises the nucleotide sequence of SEQ ID NO: 61.

In some embodiments, the RNA ligand is capable of adopting a double-stranded RNA hairpin structure. In some embodiments, the RNA ligand comprises a G:U wobble base pair.

In some embodiments, the RNA ligand comprises the nucleotide sequence of SEQ ID NO: 23. In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 23 and comprising the TLR7-selective motif of SEQ ID NO: 42 and the nucleotide sequence of SEQ ID NO: 69, wherein the nucleotide sequence of SEQ ID NO: 69 is capable of base pairing with the TLR7-selective motif.

In some embodiments, the RNA ligand comprises the nucleotide sequence of SEQ ID NO: 24. In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 24 and comprising the TLR7-selective motif of SEQ ID NO: 43 and the nucleotide sequence of SEQ ID NO: 69, wherein the nucleotide sequence of SEQ ID NO: 69 is capable of base pairing with the TLR7-selective motif.

In another aspect, provided herein is an RNA ligand comprising a TLR7-selective motif comprising the nucleotide sequence of fC-U-C or mC-U-C. In some embodiments, the RNA ligand further comprises a first nucleotide sequence linked to the 5′ end of the TLR7-selective motif. In some embodiments, the RNA ligand further comprises a second nucleotide sequence linked to the 3′ end of the TLR7-selective motif. In some embodiments, the first nucleotide sequence does not comprise a uridine nucleoside. In some embodiments, the first nucleotide sequence is linked to the TLR7-selective motif by a phosphodiester linkage. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises three nucleosides. In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises the nucleotide sequence C-G-G, wherein “-” represents a phosphodiester linkage. In some embodiments, the second nucleotide sequence does not comprise a uridine nucleoside. In some embodiments, the second nucleotide sequence comprises a nucleotide sequence that is capable of hybridizing to all or a part of the first nucleotide sequence, and/or all or a part of the TLR7-selective motif. In some embodiments, the second nucleotide sequence comprises a nucleotide sequence that is capable of hybridizing to all or a part of the first nucleotide sequence, and/or all or a part of the TLR7-selective motif, wherein the hybridized sequences form a G:U wobble base pair. In some embodiments, the second nucleotide sequence comprises the nucleotide sequence G-G-G. In some embodiments, the nucleotide sequence G-G-G is capable of base pairing with the TLR7-selective motif. In some embodiments, the nucleotide sequence G-G-G hybridizes to the TLR7-selective motif and forms a G:U wobble base pair. In some embodiments, the second nucleotide sequence is linked to the TLR7-selective motif by a phosphodiester linkage. In some embodiments, the second nucleotide sequence comprises at least three nucleosides. In some embodiments, the second nucleotide sequence comprises any of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 15 nucleosides. In some embodiments, the nucleosides within the second nucleotide sequence are linked by phosphodiester linkages or phosphorothioate linkages. In some embodiments, the nucleosides within the second nucleotide sequence are linked by phosphodiester linkages. In some embodiments, the second nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 61 and comprising the nucleotide sequence of SEQ ID NO: 69, wherein the nucleotide sequence of SEQ ID NO: 69 is capable of base pairing with the TLR7-selective motif. In some embodiments, the second nucleotide sequence comprises the nucleotide sequence of SEQ ID NO: 61. In some embodiments, the RNA ligand is capable of adopting a double-stranded RNA hairpin structure.

In some embodiments, the RNA ligand comprises a G:U wobble base pair.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 23 and comprising the TLR7-selective motif of SEQ ID NO: 42 and the nucleotide sequence of SEQ ID NO: 69, wherein the nucleotide sequence of SEQ ID NO: 69 is capable of base pairing with the TLR7-selective motif. In some embodiments, the RNA ligand is capable of adopting a double-stranded RNA hairpin structure.

In some embodiments, the RNA ligand comprises a G:U wobble base pair.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 24 and comprising the TLR7-selective motif of SEQ ID NO: 43 and the nucleotide sequence of SEQ ID NO: 69, wherein the nucleotide sequence of SEQ ID NO: 69 is capable of base pairing with the TLR7-selective motif. In some embodiments, the RNA ligand is capable of adopting a double-stranded RNA hairpin structure.

In some embodiments, the RNA ligand comprises a G:U wobble base pair.

In another aspect, provided herein is an RNA ligand comprising the nucleotide sequence of SEQ ID NOs: 23 or 24. In some embodiments, the RNA ligand is capable of adopting a double-stranded RNA hairpin structure. In some embodiments, the RNA ligand comprises a G:U wobble base pair.

RNA Ligands Comprising TLR7-selective Motif of Formula IV

In one aspect, provided herein are RNA ligands comprising one or more TLR7-selective motifs comprising Formula IV: C₁—U₂-U₃-C₄ (Formula IV), wherein C₁ and C₄ are cytidine nucleosides and U₂ and U₃ are uridine nucleosides, and “-” represents a phosphodiester linkage.

In some embodiments, the RNA ligand comprises a first and a second TLR7-selective motif comprising Formula IV.

In some embodiments, U₂ of the first TLR7-selective motif comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification.

In some embodiments, U₂ of the second TLR7-selective motif comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification.

In some embodiments, U₂ of the first TLR7-selective motif comprises a 2′O-methyl modification, and U₂ of the second TLR7-selective motif comprises a 2′O-methyl modification. In some embodiments, U₂ of the first TLR7-selective motif comprises a 2′O-methyl modification, and U₂ of the second TLR7-selective motif comprises a 2′-fluoro modification. In some embodiments, U₂ of the first TLR7-selective motif comprises a 2′O-methyl modification, and U₂ of the second TLR7-selective motif comprises a 2′-amino modification. In some embodiments, U₂ of the first TLR7-selective motif comprises a 2′O-methyl modification, and U₂ of the second TLR7-selective motif comprises a 2′-deoxy modification. In some embodiments, U₂ of the first TLR7-selective motif comprises a 2′O-methyl modification, and U₂ of the second TLR7-selective motif comprises a 2′-methoxyethoxy modification. In some embodiments, U₂ of the first TLR7-selective motif comprises a 2′-fluoro modification, and U₂ of the second TLR7-selective motif comprises a 2′O-methyl modification. In some embodiments, U₂ of the first TLR7-selective motif comprises a 2′-fluoro modification, and U₂ of the second TLR7-selective motif comprises a 2′-fluoro modification. In some embodiments, U₂ of the first TLR7-selective motif comprises a 2′-fluoro modification, and U₂ of the second TLR7-selective motif comprises a 2′-amino modification. In some embodiments, U₂ of the first TLR7-selective motif comprises a 2′-fluoro modification, and U₂ of the second TLR7-selective motif comprises a 2′-deoxy modification. In some embodiments, U₂ of the first TLR7-selective motif comprises a 2′-fluoro modification, and U₂ of the second TLR7-selective motif comprises a 2′-methoxyethoxy modification. In some embodiments, U₂ of the first TLR7-selective motif comprises a 2′-amino modification, and U₂ of the second TLR7-selective motif comprises a 2′O-methyl modification. In some embodiments, U₂ of the first TLR7-selective motif comprises a 2′-amino modification, and U₂ of the second TLR7-selective motif comprises a 2′-fluoro modification. In some embodiments, U₂ of the first TLR7-selective motif comprises a 2′-amino modification, and U₂ of the second TLR7-selective motif comprises a 2′-amino modification. In some embodiments, U₂ of the first TLR7-selective motif comprises a 2′-amino modification, and U₂ of the second TLR7-selective motif comprises a 2′-deoxy modification. In some embodiments, U₂ of the first TLR7-selective motif comprises a 2′-amino modification, and U₂ of the second TLR7-selective motif comprises a 2′-methoxyethoxy modification. In some embodiments, U₂ of the first TLR7-selective motif comprises a 2′-deoxy modification, and U₂ of the second TLR7-selective motif comprises a 2′O-methyl modification. In some embodiments, U₂ of the first TLR7-selective motif comprises a 2′-deoxy modification, and U₂ of the second TLR7-selective motif comprises a 2′-fluoro modification. In some embodiments, U₂ of the first TLR7-selective motif comprises a 2′-deoxy modification, and U₂ of the second TLR7-selective motif comprises a 2′-amino modification. In some embodiments, U₂ of the first TLR7-selective motif comprises a 2′-deoxy modification, and U₂ of the second TLR7-selective motif comprises a 2′-deoxy modification. In some embodiments, U₂ of the first TLR7-selective motif comprises a 2′-deoxy modification, and U₂ of the second TLR7-selective motif comprises a 2′-methoxyethoxy modification. In some embodiments, U₂ of the first TLR7-selective motif comprises a 2′-methoxyethoxy modification, and U₂ of the second TLR7-selective motif comprises a 2′O-methyl modification. In some embodiments, U₂ of the first TLR7-selective motif comprises a 2′-methoxyethoxy modification, and U₂ of the second TLR7-selective motif comprises a 2′-fluoro modification. In some embodiments, U₂ of the first TLR7-selective motif comprises a 2′-methoxyethoxy modification, and U₂ of the second TLR7-selective motif comprises a 2′-amino modification. In some embodiments, U₂ of the first TLR7-selective motif comprises a 2′-methoxyethoxy modification, and U₂ of the second TLR7-selective motif comprises a 2′-deoxy modification. In some embodiments, U₂ of the first TLR7-selective motif comprises a 2′-methoxyethoxy modification, and U₂ of the second TLR7-selective motif comprises a 2′-methoxyethoxy modification.

In some embodiments, the first TLR7-selective motif comprises the nucleotide sequence of SEQ ID NO: 36. In some embodiments, the second TLR7-selective motif comprises the nucleotide sequence of SEQ ID NO: 36. In some embodiments, the first TLR7-selective motif comprises the nucleotide sequence of SEQ ID NO: 37. In some embodiments, the second TLR7-selective motif comprises the nucleotide sequence of SEQ ID NO: 37. In some embodiments, the first TLR7-selective motif comprises the nucleotide sequence of SEQ ID NO: 36 and the second TLR7-selective motif comprises the nucleotide sequence of SEQ ID NO: 37. In some embodiments, the first TLR7-selective motif comprises the nucleotide sequence of SEQ ID NO: 37 and the second TLR7-selective motif comprises the nucleotide sequence of SEQ ID NO: 36.

In some embodiments, the RNA ligand further comprises one or more additional nucleotide sequences that flank the 5′ end of the TLR7-selective motifs, the 3′ end of the TLR7-selective motifs, or both. In some embodiments, the additional nucleotide sequences do not comprise a uridine nucleoside.

In some embodiments, the RNA ligand further comprises a first nucleotide sequence linked to the 5′ end of the first TLR7-selective motif. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises five nucleosides. In some embodiments, the first nucleotide sequence does not comprise a uridine nucleoside. In some embodiments, the first nucleotide sequence is linked to the first TLR7-selective motif by a phosphodiester linkage. In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises the nucleotide sequence C-A-G-A-C, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 67, wherein “-” represents a phosphodiester linkage.

In some embodiments, the RNA ligand further comprises a second nucleotide sequence linked to the 3′ end of the first TLR7-selective motif and to the 5′ end of the second TLR7-selective motif. In some embodiments, the second nucleotide sequence comprises at least one nucleoside. In some embodiments, the second nucleotide sequence comprises at least two nucleosides. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises five nucleosides. In some embodiments, the second nucleotide sequence does not comprise a uridine nucleoside. In some embodiments, the second nucleotide sequence is linked to the first and/or to the second TLR7-selective motif by a phosphodiester linkage. In some embodiments, the nucleosides within the second nucleotide sequence are linked by phosphodiester linkages or phosphorothioate linkages. In some embodiments, the nucleosides within the second nucleotide sequence are linked by phosphodiester linkages. In some embodiments, the second nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 67.

In some embodiments, the second nucleotide sequence comprises the nucleotide sequence of SEQ ID NO: 67.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 33 and comprising a first TLR7-selective motif of SEQ ID NO: 36 and/or a second TLR7-selective motif of SEQ ID NO: 36. In some embodiments, the RNA ligand comprises the nucleotide sequence of SEQ ID NO: 33. In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 34 and comprising a first TLR7-selective motif of SEQ ID NO: 37 and/or a second TLR7-selective motif of SEQ ID NO: 37.

In another aspect, provided herein is an RNA ligand comprising a first TLR7-selective motif comprising the nucleotide sequence of SEQ ID NO: 36 or 37. In some embodiments, the RNA ligand further comprises a second TLR7-selective motif comprising the nucleotide sequence of SEQ ID NO: 36 or 37. In some embodiments, the RNA ligand further comprises a first nucleotide sequence linked to the 5′ end of the first TLR7-selective motif. In some embodiments, the RNA ligand further comprises and a second nucleotide sequence linked to the 3′ end of the first TLR7-selective motif and to the 5′ end of the second TLR7-selective motif. In some embodiments, the RNA ligand comprises a first TLR7-selective motif comprising the nucleotide sequence of SEQ ID NO: 36 and a second TLR7-selective motif comprising the nucleotide sequence of SEQ ID NO: 36. In some embodiments, the RNA ligand comprises a first TLR7-selective motif comprising the nucleotide sequence of SEQ ID NO: 36 and a second TLR7-selective motif comprising the nucleotide sequence of SEQ ID NO: 37. In some embodiments, the RNA ligand comprises a first TLR7-selective motif comprising the nucleotide sequence of SEQ ID NO: 37 and a second TLR7-selective motif comprising the nucleotide sequence of SEQ ID NO: 37. In some embodiments, the RNA ligand comprises a first TLR7-selective motif comprising the nucleotide sequence of SEQ ID NO: 37 and a second TLR7-selective motif comprising the nucleotide sequence of SEQ ID NO: 36. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises five nucleosides. In some embodiments, the first nucleotide sequence does not comprise a uridine nucleoside. In some embodiments, the first nucleotide sequence is linked to the first TLR7-selective motif by a phosphodiester linkage.

In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises the nucleotide sequence C-A-G-A-C, or a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 67, wherein “-” represents a phosphodiester linkage. In some embodiments, the second nucleotide sequence comprises at least one nucleoside. In some embodiments, the second nucleotide sequence comprises at least two nucleosides. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises five nucleosides. In some embodiments, the second nucleotide sequence does not comprise a uridine nucleoside. In some embodiments, the second nucleotide sequence is linked to the first and/or to the second TLR7-selective motif by a phosphodiester linkage. In some embodiments, the nucleosides within the second nucleotide sequence are linked by phosphodiester linkages or phosphorothioate linkages. In some embodiments, the nucleosides within the second nucleotide sequence are linked by phosphodiester linkages. In some embodiments, the second nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 67. In some embodiments, the second nucleotide sequence comprises the nucleotide sequence of SEQ ID NO: 67.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 33 and comprising a first and a second TLR7-selective motif of SEQ ID NO: 36.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 34 and comprising a first and a second TLR7-selective motif of SEQ ID NO: 37

In another aspect, provided herein is an RNA ligand comprising a nucleotide sequence of SEQ ID NO: 33. In another aspect, provided herein is an RNA ligand comprising a nucleotide sequence of SEQ ID NO: 34.

RNA Ligands Comprising TLR7-selective Motif of Formula V

In one aspect, provided herein are RNA ligands comprising one or more TLR7-selective motifs comprising Formula V: U₁—U₂-C₃ (Formula V), wherein U₁ and U₂ are uridine nucleosides and C₃ is a cytidine nucleoside, and “-” represents a phosphodiester linkage.

In some embodiments, the RNA ligand comprises a first TLR7-selective motif comprising Formula V and one or more additional TLR7-selective motifs comprising Formula V. In some embodiments, the RNA ligand comprises a first TLR7-selective motif comprising Formula V and 1, 2, 3, 4, 5, 6, 7, 8, 9, 10 or more additional TLR7-selective motifs comprising Formula V. In some embodiments, the RNA ligand comprises a first TLR7-selective motif comprising Formula V and five additional TLR7-selective motifs comprising Formula V.

In some embodiments, the 3′ end of the first TLR7-selective motif is linked to the 5′ end of a second TLR7-selective motif; the 3′ of the second TLR7-selective motif is linked to the 5′ end of a third TLR7-selective motif; the 3′ of the third TLR7-selective motif is linked to the 5′ end of a fourth TLR7-selective motif; the 3′ of the fourth TLR7-selective motif is linked to the 5′ end of a fifth TLR7-selective motif; and the 3′ of the fifth TLR7-selective motif is linked to the 5′ end of a sixth TLR7-selective motif.

In some embodiments, U₁ of the one or more TLR7-selective motifs comprising Formula V comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′-methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′-methoxyethoxy modification. In some embodiments, each TLR7-selective motif comprising Formula V within the RNA ligand comprises the same modification at the 2′ position of the ribose. In some embodiments, each TLR7-selective motif comprising Formula V within the RNA ligand may comprise any of the modifications at 2′ position of the ribose described herein or known in the art in any combination. In some embodiments, U₁ of the first TLR7-selective motif comprises any modification at the 2′ position of the ribose known in the art or described herein. In some embodiments, U₁ of the first TLR7-selective motif comprises a 2′O-methyl modification, a 2′-fluoro modification, a 2′-amino modification, a 2′-deoxy modification, or a 2′-methoxyethoxy modification. In some embodiments, U₁ of a second TLR7-selective motif and of any one or more additional TLR7-selective motifs within the RNA ligand comprise any modification at the 2′ position of the ribose known in the art or described herein. In some embodiments, U₁ of a second TLR7-selective motif and of any one or more additional TLR7-selective motifs within the RNA ligand comprise a 2′O-methyl modification, a 2′-fluoro modification, a 2′-amino modification, a 2′-deoxy modification, or a 2′-methoxyethoxy modification.

In some embodiments, the RNA ligand comprises six TLR7-selective motifs comprising Formula V. In some embodiments, U₁ of the first TLR7-selective motif comprises any modification at the 2′ position of the ribose known in the art or described herein. In some embodiments, U₁ of the first TLR7-selective motif comprises a 2′O-methyl modification, a 2′-fluoro modification, a 2′-amino modification, a 2′-deoxy modification, or a 2′-methoxyethoxy modification. In some embodiments, U₁ of the second TLR7-selective motif comprises any modification at the 2′ position of the ribose known in the art or described herein. In some embodiments, U₁ of the second TLR7-selective motif comprises a 2′O-methyl modification, a 2′-fluoro modification, a 2′-amino modification, a 2′-deoxy modification, or a 2′-methoxyethoxy modification. In some embodiments, U₁ of the third TLR7-selective motif comprises any modification at the 2′ position of the ribose known in the art or described herein. In some embodiments, U₁ of the third TLR7-selective motif comprises a 2′O-methyl modification, a 2′-fluoro modification, a 2′-amino modification, a 2′-deoxy modification, or a 2′-methoxyethoxy modification. In some embodiments, U₁ of the fourth TLR7-selective motif comprises any modification at the 2′ position of the ribose known in the art or described herein. In some embodiments, U₁ of the fourth TLR7-selective motif comprises a 2′O-methyl modification, a 2′-fluoro modification, a 2′-amino modification, a 2′-deoxy modification, or a 2′-methoxyethoxy modification. In some embodiments, U₁ of the fifth TLR7-selective motif comprises any modification at the 2′ position of the ribose known in the art or described herein. In some embodiments, U₁ of the fifth TLR7-selective motif comprises a 2′O-methyl modification, a 2′-fluoro modification, a 2′-amino modification, a 2′-deoxy modification, or a 2′-methoxyethoxy modification. In some embodiments, U₁ of the sixth TLR7-selective motif comprises any modification at the 2′ position of the ribose known in the art or described herein. In some embodiments, U₁ of the sixth TLR7-selective motif comprises a 2′O-methyl modification, a 2′-fluoro modification, a 2′-amino modification, a 2′-deoxy modification, or a 2′-methoxyethoxy modification.

In some embodiments, the RNA ligand comprises six TLR7-selective motifs comprising Formula V. In some embodiments, U₁ of the first TLR7-selective motif comprises a 2′O-methyl modification, U₁ of the second TLR7-selective motif comprises a 2′O-methyl modification, U₁ of the third TLR7-selective motif comprises a 2′O-methyl modification, U₁ of the fourth TLR7-selective motif comprises a 2′O-methyl modification, U₁ of the fifth TLR7-selective motif comprises a 2′O-methyl modification, and U₁ of the sixth TLR7-selective motif comprises a 2′O-methyl modification.

In some embodiments, the RNA ligand comprises six TLR7-selective motifs comprising Formula V. In some embodiments, U₁ of the first TLR7-selective motif comprises a 2′-fluoro modification, U₁ of the second TLR7-selective motif comprises a 2′-fluoro modification, U₁ of the third TLR7-selective motif comprises a 2′-fluoro modification, U₁ of the fourth TLR7-selective motif comprises a 2′-fluoro modification, U₁ of the fifth TLR7-selective motif comprises a 2′-fluoro modification, and U₁ of the sixth TLR7-selective motif comprises a 2′-fluoro modification.

In some embodiments, the first TLR7-selective motif comprises the nucleotide sequence of SEQ ID NO: 40. In some embodiments, at least one of one or more additional TLR7-selective motifs (e.g., any of 1, 2, 3, 4, 5, 6, 7, 8, 9, 10 or more additional TLR7-selective motifs), or all of one or more additional TLR7-selective motifs (e.g., any of 1, 2, 3, 4, 5, 6, 7, 8, 9, 10 or more additional TLR7-selective motifs) comprise the nucleotide sequence of SEQ ID NO: 40.

In some embodiments, the first TLR7-selective motif comprises the nucleotide sequence of SEQ ID NO: 41. In some embodiments, at least one of one or more additional TLR7-selective motifs (e.g., any of 1, 2, 3, 4, 5, 6, 7, 8, 9, 10 or more additional TLR7-selective motifs), or all of one or more additional TLR7-selective motifs (e.g., any of 1, 2, 3, 4, 5, 6, 7, 8, 9, 10 or more additional TLR7-selective motifs) comprise the nucleotide sequence of SEQ ID NO: 41.

In some embodiments, the first TLR7-selective motif comprises the nucleotide sequence of SEQ ID NO: 40. In some embodiments, the RNA ligand comprises one or more additional TLR7-selective motifs (e.g., any of 1, 2, 3, 4, 5, 6, 7, 8, 9, 10 or more additional TLR7-selective motifs) comprising any combination of the nucleotide sequence of SEQ ID NO: 40 and SEQ ID NO: 41.

In some embodiments, the first TLR7-selective motif comprises the nucleotide sequence of SEQ ID NO: 41. In some embodiments, the RNA ligand comprises one or more additional TLR7-selective motifs (e.g., any of 1, 2, 3, 4, 5, 6, 7, 8, 9, 10 or more additional TLR7-selective motifs) comprising any combination of the nucleotide sequence of SEQ ID NO: 40 and SEQ ID NO: 41.

In some embodiments, the RNA ligand further comprises one or more additional nucleotide sequences that flank the 5′ end of one or more of the TLR7-selective motifs, the 3′ end of one or more of the TLR7-selective motifs, or both. In some embodiments, the additional nucleotide sequences do not comprise a uridine nucleoside.

In some embodiments, the RNA ligand comprises a first nucleotide sequence linked to the 5′ end of the first TLR7-selective motif. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises two nucleosides. In some embodiments, the first nucleotide sequence does not comprise a uridine nucleoside. In some embodiments, the first nucleotide sequence is linked to the first TLR7-selective motif by a phosphodiester linkage. In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises two cytidine nucleosides.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 29 and comprising a first TLR7-selective motif of SEQ ID NO: 40 and five additional TLR7-selective motifs of SEQ ID NO: 40. In some embodiments, the RNA ligand comprises the nucleotide sequence of SEQ ID NO: 29.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 30 and comprising a first TLR7-selective motif of SEQ ID NO: 41 and five additional TLR7-selective motifs of SEQ ID NO: 41. In some embodiments, the RNA ligand comprises the nucleotide sequence of SEQ ID NO: 30.

In another aspect, provided herein is an RNA ligand comprising a first TLR7-selective motif comprising the nucleotide sequence of SEQ ID NO: 40 or 41. In some embodiments, the RNA ligand comprises one or more additional TLR7-selective motifs, wherein at least one of the one or more additional TLR7-selective motifs, or all of the one or more additional TLR7-selective motifs comprise the nucleotide sequence of SEQ ID NO: 40 or 41. In some embodiments, the RNA ligand comprises a first nucleotide sequence linked to the 5′ end of the first TLR7-selective motif. In some embodiments, the RNA ligand comprises a first TLR7-selective motif and 1, 2, 3, 4, 5, 6, 7, 8, 9, 10 or more additional TLR7-selective motifs. In some embodiments, the RNA ligand comprises a first TLR7-selective motif and five additional TLR7-selective motifs. In some embodiments, the TLR7-selective motifs within the RNA ligand comprise any combination of the nucleotide sequence of SEQ ID NO: 40 and SEQ ID NO: 41. In some embodiments, the TLR7-selective motifs within the RNA ligand comprise the nucleotide sequence of SEQ ID NO: 40. In some embodiments, the TLR7-selective motifs within the RNA ligand comprise the nucleotide sequence of SEQ ID NO: 41. In some embodiments, the first nucleotide sequence comprises at least one nucleoside. In some embodiments, the first nucleotide sequence comprises at least two nucleosides. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises two nucleosides. In some embodiments, the first nucleotide sequence does not comprise a uridine nucleoside. In some embodiments, the first nucleotide sequence is linked to the first TLR7-selective motif by a phosphodiester linkage. In some embodiments, the nucleosides within the first nucleotide sequence are linked by phosphodiester linkages or phosphorothioate linkages. In some embodiments, the nucleosides within the first nucleotide sequence are linked by phosphodiester linkages. In some embodiments, the first nucleotide sequence comprises two cytidine nucleosides.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 29 and comprising six TLR7-selective motifs of SEQ ID NO: 40.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 30 and comprising six TLR7-selective motifs of SEQ ID NO: 41.

In another aspect, provided herein is an RNA ligand comprising a nucleotide sequence of SEQ ID NOs: 29 or 30. In some embodiments, the RNA ligand comprises the nucleotide sequence of SEQ ID NO: 29. In some embodiments, the RNA ligand comprises the nucleotide sequence of SEQ ID NO: 30.

RNA Ligands Comprising TLR7-Selective Motif of Formula VI

In one aspect, provided herein are RNA ligands comprising a TLR7-selective motif comprising Formula VI:

C₁-U₂-U₃*U₄-U₅*C₆ (Formula VI),

wherein C₁ and C₆ are cytidine nucleosides, and U₂, U₃, U₄, and U₅ are uridine nucleosides, wherein “*” represents a phosphorothioate linkage and “-” represents a phosphodiester linkage.

In some embodiments, U₂ comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′methoxyethoxy modification.

In some embodiments, U₄ comprises a modification at the 2′ position of the ribose. In some embodiments, the modification is any modification at the 2′ position of the ribose known in the art or described herein. In some embodiments, the modification is any modification at the 2′ position of the ribose that inhibits or blocks RNase activity known in the art or described herein. In some embodiments, the modification is a 2′O-methyl modification, a 2′-fluoro modification, a 2′-amino modification, a 2′-deoxy modification, or a 2′methoxyethoxy modification. In some embodiments, the modification is a 2′O-methyl modification. In some embodiments, the modification is a 2′-fluoro modification. In some embodiments, the modification is a 2′-amino modification. In some embodiments, the modification is a 2′-deoxy modification. In some embodiments, the modification is a 2′methoxyethoxy modification.

In some embodiments, the ribose modification of U₂ is a 2′O-methyl modification, and the ribose modification of U₄ is a 2′O-methyl modification. In some embodiments, the ribose modification of U₂ is a 2′O-methyl modification, and the ribose modification of U₄ is a 2′-fluoro modification. In some embodiments, the ribose modification of U₂ is a 2′O-methyl modification, and the ribose modification of U₄ is a 2′-amino modification. In some embodiments, the ribose modification of U₂ is a 2′O-methyl modification, and the ribose modification of U₄ is a 2′-deoxy modification. In some embodiments, the ribose modification of U₂ is a 2′O-methyl modification, and the ribose modification of U₄ is a 2′methoxyethoxy modification. In some embodiments, the ribose modification of U₂ is a 2′-fluoro modification, and the ribose modification of U₄ is a 2′O-methyl modification. In some embodiments, the ribose modification of U₂ is a 2′-fluoro modification, and the ribose modification of U₄ is a 2′-fluoro modification. In some embodiments, the ribose modification of U₂ is a 2′-fluoro modification, and the ribose modification of U₄ is a 2′-amino modification. In some embodiments, the ribose modification of U₂ is a 2′-fluoro modification, and the ribose modification of U₄ is a 2′-deoxy modification. In some embodiments, the ribose modification of U₂ is a 2′-fluoro modification, and the ribose modification of U₄ is a 2′methoxyethoxy modification. In some embodiments, the ribose modification of U₂ is a 2′-amino modification, and the ribose modification of U₄ is a 2′O-methyl modification. In some embodiments, the ribose modification of U₂ is a 2′-amino modification, and the ribose modification of U₄ is a 2′-fluoro modification. In some embodiments, the ribose modification of U₂ is a 2′-amino modification, and the ribose modification of U₄ is a 2′-amino modification. In some embodiments, the ribose modification of U₂ is a 2′-amino modification, and the ribose modification of U₄ is a 2′-deoxy modification. In some embodiments, the ribose modification of U₂ is a 2′-amino modification, and the ribose modification of U₄ is a 2′-methoxyethoxy modification. In some embodiments, the ribose modification of U₂ is a 2′-deoxy modification, and the ribose modification of U₄ is a 2′O-methyl modification. In some embodiments, the ribose modification of U₂ is a 2′-deoxy modification, and the ribose modification of U₄ is a 2′-fluoro modification. In some embodiments, the ribose modification of U₂ is a 2′-deoxy modification, and the ribose modification of U₄ is a 2′-amino modification. In some embodiments, the ribose modification of U₂ is a 2′-deoxy modification, and the ribose modification of U₄ is a 2′-deoxy modification. In some embodiments, the ribose modification of U₂ is a 2′-deoxy modification, and the ribose modification of U₄ is a 2′methoxyethoxy modification. In some embodiments, the ribose modification of U₂ is a 2′methoxyethoxy modification, and the ribose modification of U₄ is a 2′O-methyl modification. In some embodiments, the ribose modification of U₂ is a 2′methoxyethoxy modification, and the ribose modification of U₄ is a 2′-fluoro modification. In some embodiments, the ribose modification of U₂ is a 2′methoxyethoxy modification, and the ribose modification of U₄ is a 2′-amino modification. In some embodiments, the ribose modification of U₂ is a 2′methoxyethoxy modification, and the ribose modification of U₄ is a 2′-deoxy modification. In some embodiments, the ribose modification of U₂ is a 2′methoxyethoxy modification, and the ribose modification of U₄ is a 2′methoxyethoxy modification.

In some embodiments, the TLR7-selective motif comprises the nucleotide sequence of SEQ ID NO: 48. In some embodiments, the TLR7-selective motif comprises the nucleotide sequence of SEQ ID NO: 49. In some embodiments, the TLR7-selective motif comprises the nucleotide sequence of SEQ ID NO: 50. In some embodiments, the TLR7-selective motif comprises the nucleotide sequence of SEQ ID NO: 51.

In some embodiments, the RNA ligand further comprises additional nucleotide sequences that flank the 5′ end of the TLR7-selective motif, the 3′ end of the TLR7-selective motif, or both. In some embodiments, the additional nucleotide sequences do not comprise a uridine nucleoside.

In some embodiments, the RNA ligand further comprises a first nucleotide sequence that is linked to the 5′ end of the TLR7-selective motif. In some embodiments, the first nucleotide sequence comprises at least one nucleoside. In some embodiments, the first nucleotide sequence comprises at least two nucleosides. In some embodiments, the nucleosides of the first nucleotide sequence are linked by phosphodiester linkages or phosphorothioate linkages. In some embodiments, the nucleosides of the first nucleotide sequence are linked by phosphodiester linkages. In some embodiments, the first nucleotide sequence does not comprise a uridine nucleoside. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises 8 nucleosides. In some embodiments, the first nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 68. In some embodiments, the first nucleotide sequence comprises the nucleotide sequence of SEQ ID NO: 68. In some embodiments, the first nucleotide sequence is linked to the 5′ end of the TLR7-selective motif by a phosphodiester linkage or a phosphorothioate linkage. In some embodiments, the first nucleotide sequence is linked to the 5′ end of the TLR7-selective motif by a phosphodiester linkage.

In some embodiments, the RNA ligand further comprises a second nucleotide sequence that is linked to the 3′ end of the TLR7-selective motif. In some embodiments, the second nucleotide sequence comprises at least one nucleoside. In some embodiments, the second nucleotide sequence comprises at least two nucleosides. In some embodiments, the nucleosides of the second nucleotide sequence are linked by phosphodiester linkages or phosphorothioate linkages. In some embodiments, the nucleosides of the second nucleotide sequence are linked by phosphodiester linkages. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 2 nucleosides. In some embodiments, the second nucleotide sequence does not comprise a uridine nucleoside. In some embodiments, the second nucleotide sequence comprises two cytidine nucleosides. In some embodiments, the second nucleotide sequence is linked to the 3′ end of the TLR7-selective motif by a phosphodiester linkage or a phosphorothioate linkage. In some embodiments, the second nucleotide sequence is linked to the 3′ end of the TLR7-selective motif by a phosphodiester linkage.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 17 and comprising the TLR7-selective motif of SEQ ID NO: 48. In some embodiments, the RNA ligand comprises the nucleotide sequence of SEQ ID NO: 17.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 18 and comprising the TLR7-selective motif of SEQ ID NO: 49. In some embodiments, the RNA ligand comprises the nucleotide sequence of SEQ ID NO: 18.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 19 and comprising the TLR7-selective motif of SEQ ID NO: 50. In some embodiments, the RNA ligand comprises the nucleotide sequence of SEQ ID NO: 19.

In some embodiments, the RNA ligand comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 20 and comprising the TLR7-selective motif of SEQ ID NO: 51. In some embodiments, the RNA ligand comprises the nucleotide sequence of SEQ ID NO: 20.

In another aspect, provided herein is an RNA ligand comprising a TLR7-selective motif comprising a nucleotide sequence of C-mU-U*mU-U*C, C-fU-U*fU-U*C, C-mU-U*fU-U*C, and C-fU-U*mU-U*C. In some embodiments, the RNA ligand further comprises a first nucleotide sequence linked to the 5′ end of the TLR7-selective motif. In some embodiments, the RNA ligand further comprises a second nucleotide sequence linked to the 3′ end of the TLR7-selective motif. In some embodiments, the first nucleotide sequence comprises at least one nucleoside. In some embodiments, the first nucleotide sequence comprises at least two nucleosides. In some embodiments, the nucleosides of the first nucleotide sequence are linked by phosphodiester linkages or phosphorothioate linkages. In some embodiments, the nucleosides of the first nucleotide sequence are linked by phosphodiester linkages. In some embodiments, the first nucleotide sequence does not comprise a uridine nucleoside. In some embodiments, the first nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the first nucleotide sequence comprises 8 nucleosides. In some embodiments, the first nucleotide sequence comprises a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 68. In some embodiments, the first nucleotide sequence comprises the nucleotide sequence of SEQ ID NO: 68. In some embodiments, the first nucleotide sequence is linked to the 5′ end of the TLR7-selective motif by a phosphodiester linkage or a phosphorothioate linkage. In some embodiments, the first nucleotide sequence is linked to the 5′ end of the TLR7-selective motif by a phosphodiester linkage. In some embodiments, the second nucleotide sequence comprises at least one nucleoside. In some embodiments, the second nucleotide sequence comprises at least two nucleosides. In some embodiments, the nucleosides of the second nucleotide sequence are linked by phosphodiester linkages or phosphorothioate linkages. In some embodiments, the nucleosides of the second nucleotide sequence are linked by phosphodiester linkages. In some embodiments, the second nucleotide sequence comprises any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 94, 96, 97, 98, 99, 100, or more nucleosides, or between any of 100 and 125 nucleosides, 125 and 150 nucleosides, 150 and 175 nucleosides, 175 and 200 nucleosides, 200 and 225 nucleosides, 225 and 250 nucleosides, 250 and 275 nucleosides, or 275 and 300 nucleosides. In some embodiments, the second nucleotide sequence comprises 2 nucleosides. In some embodiments, the second nucleotide sequence does not comprise a uridine nucleoside. In some embodiments, the second nucleotide sequence comprises two cytidine nucleosides. In some embodiments, the second nucleotide sequence is linked to the 3′ end of the TLR7-selective motif by a phosphodiester linkage or a phosphorothioate linkage. In some embodiments, the second nucleotide sequence is linked to the 3′ end of the TLR7-selective motif by a phosphodiester linkage.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 17 and comprising the TLR7-selective motif of SEQ ID NO: 48. In some embodiments, the RNA ligand comprises the nucleotide sequence of SEQ ID NO: 17.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 18 and comprising the TLR7-selective motif of SEQ ID NO: 49. In some embodiments, the RNA ligand comprises the nucleotide sequence of SEQ ID NO: 18.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 19 and comprising the TLR7-selective motif of SEQ ID NO: 50. In some embodiments, the RNA ligand comprises the nucleotide sequence of SEQ ID NO: 19.

In another aspect, provided herein is an RNA ligand comprising a nucleotide sequence having at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to SEQ ID NO: 20 and comprising the TLR7-selective motif of SEQ ID NO: 51. In some embodiments, the RNA ligand comprises the nucleotide sequence of SEQ ID NO: 20.

RNA Modifications

In some aspects, provide herein are RNA ligands that comprise a TLR7-selective motif comprising one or more modifications of at least one nucleotide. The one or more nucleotide modifications may be a modified base, a modified sugar, and/or a modified phosphate. In some embodiments, the one or more nucleotide modifications may be a phosphate-modified linkage. In some embodiments, the one or more modifications interfere with RNase activity, e.g., endoribonuclease or exoribonuclease activity. In some embodiments, the one or more modifications inhibit or block RNase activity or RNase binding to the RNA ligand.

Nucleobase Modifications

In some embodiments, an RNA ligand of the disclosure comprises a TLR7-selective motif comprising at least one modified base.

In some embodiments, the modified base is a modified a purine or a modified pyrimidine. In some embodiments, the modified base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g., 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N⁶-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid and 2-thiocytidine.

In some embodiments, an RNA ligand of the disclosure comprises a TLR7-selective motif comprising at least one abasic nucleotide. In some embodiments, the abasic nucleotide comprises a chemical group other than a base at the 1′ position, e.g., a 3′,3′-linked or 5′,5′-linked deoxyabasic ribose derivative.

Sugar Moiety Modifications

In some embodiments, an RNA ligand of the disclosure comprises a TLR7-selective motif comprising at least one nucleotide with a modified sugar moiety. In some embodiments, the modified sugar moiety is a modified ribose moiety.

In some embodiments, RNA ligands of the disclosure may include analogous forms of ribose that are generally known in the art, such as, without limitation, carbocyclic sugar analogs, a-anomeric sugars, epimeric sugars such as arabinose, xyloses or lyxoses, pyranose sugars, furanose sugars, sedoheptuloses, acyclic analogs, and basic nucleoside analogs such as methyl riboside.

In some embodiments, the modified ribose moiety comprises a modification at the 2′ position. In some embodiments, the 2′-OH of the ribose is substituted with another moiety, such as alkyl, substituted alkyl, alkaryl-, arylalkyl-, —F, —Cl, —Br, —CN, —CF₃, —OCF₃, —OCN, —O-alkyl, —S-alkyl, HS-alkyl-O, —O-alkenyl, —S-alkenyl, —N-alkenyl, —SO-alkyl, -alkyl-OSH, -alkyl-OH, —O— alkyl-OH, —O-alkyl-SH, —S-alkyl-OH, —S-alkyl-SH, -alkyl-S-alkyl, -alkyl-O-alkyl, —ONO₂, —NO₂, —N₃,-NH₂, alkylamino, dialkylamino-, aminoalkyl-, aminoalkoxy, aminoacid, aminoacyl-, —ONH₂, —O-aminoalkyl, —O-aminoacid,-O-aminoacyl, heterocycloalkyl-, heterocycloalkaryl-, aminoalkylamino-, polyalklylamino-, substituted silyl-, methoxyethyl-(MOE), alkenyl or alkynyl. Other exemplary 2′ ribose modifications include C₁ to Ci₀ lower alkyl, substituted lower aralkyl, O-alkaryl or O-aralkyl, SH₁, SCH₃, SOCH₃, SO₂CH₃, polyalkylamino, substituted silyl, a reporter group, or an intercalator. In some embodiments, the modification at the 2′ position of the ribose is a 2′O-methyl modification, a 2′-O-allyl modification, a 2′-azido-ribose modification, a 2′-fluoro modification, a 2′-amino modification, a 2′-deoxy modification, or a 2′-methoxyethoxy modification. In some embodiments, the modification at the 2′ position of the ribose is a 2′O-methyl modification, a 2′-fluoro modification, a 2′-amino modification, a 2′-deoxy modification, or a 2′-methoxyethoxy modification. In some embodiments, the modification at the 2′ position of the ribose is a 2′O-methyl modification or a 2′-fluoro modification.

Phosphate and Linkage Modifications

In some embodiments, the one or more nucleotide modifications comprise a phosphate modification.

In some embodiments, the phosphate modification is a modified phosphodiester linkage modification. For example, one or more phosphodiester linkages may be replaced by alternative linking groups, such as, without limitation, P(O)S (“thioate”), P(S)S (“dithioate”), (O)NR2 (‘amidate”), P(O)R, P(R)OR′, CO or CH₂ (“formacetal”), in which each R or R′ is independently H or substituted or unsubstituted alkyl (1-20 C), optionally containing an ether (—O—) linkage, aryl, alkenyl, cycloaklyl, cycloalkenyl, or araldyl. Other exemplary phosphate modifications include, but are not limited to, methyl phosphonate, phosphorothioate, phosphoamidates, a carbamate, phosphoramidate, phosphotriester, or phosphorodithioate. In some embodiments, the nucleotide modification is phosphorothioate linkage modification.

In some embodiments, the phosphate modification is a 3′-terminal internucleotide phosphodiester linkage modification, such as an alkyl or aryl phosphotriester, an alkyl or aryl phosphonate, a hydrogen phosphonate, a phosphoramidate, and/or a phosphoroselenate linkage modification. In some embodiments, the 3′-terminal internucleotide phophodiester linkage modification is a phosphoramidate modification.

Not all linkages in an RNA ligand of the disclosure need be identical. For example, an RNA ligand of the disclosure may include any combination of phosphodiester, methyl phosphonate, phosphorothioate, phosphoamidates, carbamate, phosphoramidate, phosphotriester, or phosphorodithioate linkages. In some embodiments, an RNA ligand of the disclosure comprises any combination of phosphodiester and phosphorothioate linkages. In some embodiments, an RNA ligand of the disclosure comprises only phosphodiester linkages. In some embodiments, an RNA ligand of the disclosure comprises only phosphorothioate linkages.

Additional RNA Modifications

In some embodiments, RNA ligands of the disclosure may comprise modifications such as RNA caps (e.g., 7-methyl guanosine (m7G) cap, m7Gppp5′N, 2′O methylated m7G cap, m7G(5′)ppp(5′)G, m7G(5′)ppp(5′)A, G(5′)ppp(5′)A, G(5′)ppp(5′)G, 3′-O-Me-m7G(5′)ppp(5′)G, ARCA, or any other suitable RNA cap known in the art), and/or tails (e.g., poly(A) tail); pendant moieties, such as, for example, proteins (e.g., nucleases, toxins, antibodies, signal peptides, poly-L-lysine, etc.); intercalators (e.g., acridine, psoralen, etc.); chelators (e.g., metals, radioactive metals, boron, oxidative metals, etc.); alkylators, and modified linkages (e.g., alpha anomeric nucleic acids, etc.). In some embodiments, RNA ligands of the disclosure may be conjugated to solid or semi-solid supports. In some embodiments, the 5′ and 3′ terminal OH can be phosphorylated or substituted with amines or organic capping group moieties, e.g., of from 1 to 20 carbon atoms. Other hydroxyls may also be derivatized to standard protecting groups.

### Preparation of RNA Ligands

An RNA ligand of the disclosure, e.g., comprising one or more RNA modifications described herein, may be prepared using any suitable method known in the art.

In some embodiments, de novo methods for RNA synthesis may be used, including, but not limited to, chemical synthesis using suitable protecting groups (see, e.g., Masuda et al., (2007) Nucleic Acids Symposium Series 57:3-4); the b-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedronr Lett 27:4055-8; Gaffney B L et al. (1988) Tetrahedron Lett 29:2619-22); or synthetic methods disclosed in Uhlmann et al. (1990) Chem Rev 90:544-84, and Goodchild J (1990) Bioconjugate Chem 1: 165. These and other methods can be performed or adapted for use with automated nucleic acid synthesizers that are commercially available.

RNA ligands of the disclosure may also be prepared using suitable recombinant methods that are well known and conventional in the art, such as cloning, processing, and/or expression of polynucleotides in host cells, e.g., as described herein. Site-directed mutagenesis can be used to alter nucleic acids, for example, to insert new restriction sites, introduce mutations and the like. Suitable methods for transcription of nucleic acid sequences are known and conventional in the art. (See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.)

In some embodiments, RNA ligands of the disclosure may also be prepared using in vitro transcription methods. As is recognized in the art, typical in vitro RNA transcription reactions include an RNA polymerase, e.g., a T7 RNA polymerase; a DNA template; nucleotides (NTPs); ions such as magnesium, e.g., magnesium acetate; a buffer such as, e.g., HEPES or Tris, e.g., at a suitable pH, e.g., 7-8.5. In some embodiments, additional compounds such as dithiothreitol (DTT) and/or spermidine may be included. In some embodiments, an RNase inhibitor is included in the in vitro RNA transcription reaction to ensure no RNase induced degradation during the transcription reaction. In some embodiments a pyrophosphatase is included in the reaction to cleave the inorganic pyrophosphate generated following each nucleotide incorporation into two units of inorganic phosphate. This ensures that magnesium remains in solution and does not precipitate as magnesium pyrophosphate. Products of an in vitro RNA transcription reaction may be purified according to any suitable method known in the art, e.g., as described below.

Preparation of RNA ligands of the disclosure may include one or more steps of purifying or isolating the RNA ligands (e.g., from reaction mixtures, cellular components, and the like). Such purification may be accomplished using any suitable method known in the art, such as by high performance liquid chromatography (HPLC), column-based methods (e.g., mini Quick Spin DNA Columns (Roche)), polyacrylamide gel electrophoresis (PAGE), and/or RNase-Free HPLC purification.

The presence and/or quantity of one or more RNA modifications in an RNA ligand of the disclosure may be determined using any suitable method. For example, an RNA ligand of the disclosure can be digested to monophosphates (e.g., using nuclease P1) and dephosphorylated (e.g., using a suitable phosphatase such as CIAP), and the resulting nucleosides analyzed by reversed phase HPLC. Other methods that may be used include mass spectrometry (MS), e.g., matrix-assisted laser desorption/ionization (MALDI), tandem MS (MS/MS), electrospray ionization mass spectroscopy (ESI-MS), or liquid chromatography-mass spectrometry (LC-MS); chromatography methods, such as thin layer chromatography (TLC), or liquid chromatography (LC); reverse-transcription-based methods; RNase H cleavage-based methods; ligation-based methods; Northern blotting; Demethylase-facilitated RNA-Sequencing; MCi ribonuclease cleavage; direct RNA sequencing; amplification-based sequencing; and bisulfite sequencing.

### Carriers

In some embodiments, the immunomodulatory compositions of the disclosure further comprise a carrier, such as a pharmaceutically acceptable carrier.

In some embodiments, the carrier facilitates the intracellular transport of the RNA ligand into one or more cells. In some embodiments, the carrier is cationic agent, such as a cationic or polycationic compound. In some embodiments, the carrier is any carrier suitable for delivering an RNA ligand of the disclosure into one or more cells. Examples of carriers that may be used in the compositions of the disclosure include, without limitation polyethylenimine (PEI); polyalkylenimines; polyarginines, e.g., poly-L-ariginine; polyamines; protamines; polylysines, e.g., poly-L-lysine or POLL; nucleoline; fusogenic peptides; polyamidoamine dendrimers (PAMAM, Dendritech Inc.); pegylated cationic polymers; cationic polymer conjugates (e.g., PEI-cholesterol, polylysine cholesterol, etc.); spermine or spermidine; chitosans; cationic dextrans; basic polypeptides; cell penetrating peptides (CPPs), such as HIV-binding peptides, HIV-1 Tat (HIV), Tat-derived peptides, Penetratin, VP22 derived or analog peptides, Pestivirus Ems, HSV, VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB(1), pVEC, hCT-derived peptides, SAP, or histones; cationic polysaccharides, for example, chitosan; cationic cyclodextrins; polybrene; cationic or polycationic polymers, such as polyamino acids (e.g., poly-L-arginine, poly-L-lysine, poly-L-ornithine); modified polyamino acids, such as j-amino acid-polymers or reversed polyamides; modified polyethylenes, such as PVP (poly(N-ethyl-4-vinylpyridinium bromide)); modified acrylates, such as pDMAEMA (poly(dimethylaminoethyl methylacrylate)); modified amidoamines such as pAMAM (poly(amidoamine)); modified polybetaminoester (PBAE), such as diamine end modified 1,4 butanediol diacrylate-co-5-amino-1-pentanol polymers; dendrimers, such as polypropylamine dendrimers or pAMAM based dendrimers; polyimine, e.g., PEI, poly(propyleneimine); polyallylamine; sugar backbone based polymers, such as cyclodextrin based polymers, dextran based polymers, chitosan; silan backbone based polymers, such as PMOXA-PDMS copolymers; block polymers comprising a combination of one or more cationic blocks (e.g., selected from a cationic polymer as described above) and one or more hydrophilic or hydrophobic blocks (e.g., polyethylene glycol).

In some embodiments, the carrier is a lipid.

In some embodiments, the carrier is a cationic lipid. Examples of cationic lipids that may be used as carriers include, without limitation, dioleoyltrimethyl-ammonium propane (DOTAP), N-[1(-2,3-dioleoyloxy)propyl]-N,N,N-trimethylammonium chloride (DOTMA), N,N-dioleyl-N,N-dimethylammonium chloride (DODAC), Lipofectamine™, Gene Porter™, DMRIE (1,2-dimyristyloxy-propyl-3-dimethyl-hydroxy ethyl ammonium bromide), di-C₁₄-amidine, DOTIM (1-[2-(oleoyloxy)ethyl]-2-oleyl-3-(2-hydroxyeth-yl)imidazolinium chloride), dimethyldioctadecyl ammonium bromide (DDAB), DOGS, 1,2-dioleoyloxypropyl-3-dimethyl-hydroxyethylammonium bromide (DORI), N-(3-aminopro-pyl)-N,N-dimethyl-2,3-bis(dodecyloxy)-1-propanammonium bromide (GAP-DLRIE), SAINT, DC-Chol, bis-guanidinium-tren-cholesterol (BGTC), CTAP, 1,2-Dioleoyl-sn-glycero-3-phosphocholine (DOPC), 1,2-dioleoyl-3-dimethylammonium-propane (DODAP), Dioleyl phosphatidylethanol-amine (DOPE), 2,3-dioleyloxy-N-[2(sperminecarboxamido)ethyl]-N,N-dimethyl-1-propanamin-iumtrifluoro-acetate (DOSPA), Dioctadecyldimethylammonium bromide (DODAB), DOIC, DMEPC, Dimyristooxypropyl dimethyl hydroxyethyl ammonium bromide (DIMRI), O,O-ditetradecanoyl-N-a-trimethylammonioacetyl)diethanolamine chloride (DC-6-14), rac-[(2,3-dioctadecyloxypropyl)(2-hydroxyethyl)]-dimethylammonium chloride (CLIP1), rac-[2(2,3-dihexadecyloxypropyloxymethyloxy)ethyl]-trimethylammonium (CLIP6), rac-[2(2,3-dihexadecyloxypropyloxysuccinyloxy)ethyl]-trimethylammonium (CLIP9), SM-102, or oligofectamine. Other lipids that may be used as carriers include, without limitation, those described in US 2008/0085870 and US 2008/0057080, which are incorporated herein by reference.

In some embodiments, the carrier is jetPEI (see, e.g., Ami Sidi et al., The Journal of Urology (2008) 180(6):2379-2383).

In some embodiments, the carrier is an ionizable lipid. Examples of ionizable lipids that may be used as carriers include, without limitation, ALC-0315 ([(4-hydroxybutyl)azanediyl]di(hexane-6,1-diyl) bis(2-hexyldecanoate)) (see, e.g., the website: assets.publishing.service.gov.uk/government/uploads/system/uploads/attachment_data/file/94454 4/COVID-19_mRNA_Vaccine_BNT162b2__UKPAR__PFIZER_BIONTECH__15dec.2020.pdf), SM-102 (heptadecan-9-yl 8-((2-hydroxyethyl) (6-oxo-6-(undecyloxy) hexyl) amino) octanoate) (see, e.g., the website: www.modernatx.com/sites/default/files/mRNA-1273-P301-Protocol.pdf), imidazole cholesterol ester (see, e.g., US20200155691A1), a cysteine-based ionizable lipid (e.g., as disclosed in WO2020214946A1, which is incorporated herein by reference with respect to its disclosure of cysteine-based ionizable lipids), Lipid 2,2 (8,8) 4C CH3 (see, e.g., U.S. Pat. No. 9,670,152B2), Genevant CL1 (see, e.g., WO2020219941A1), Acuitas (A9) (see, e.g, Conway et al., Molecular Therapy (2019) 27(4):866-877), DLin-MC3-DMA (see, e.g., Jayaraman et al., Angew Chem Int Ed Engl (2012) 51(34):8529-8533), Lipid 5 (see, e.g., Sabnis et al., Molecular Therapy (2018) 26(6):1509-1519; and the website: investors.modernatx.com/news-releases/news-release-details/moderna-announces-positive-phase-1-results-first-systemic), C₁₂-200 (see, e.g., Love et al., PNAS (2010) 107(5):1864-1869), 5A2-SC8 (see, e.g., Cheng et al., Advanced Materials (2018) 30(52):1805308), DLin-KC2-DMA (see, e.g., Semple et al., Nature Biotechnology (2010) 28:172-176), OF-02 (see, e.g., Fenton et al., Advanced Materials (2016) 28(15):2939-2943), 3060i10 (see, e.g., Hajj et al., Small (2019) 15(6):1805097), cKK-E12 (see, e.g., Dong et al., PNAS (2014) 111(11):3955-3960), SS-EC, NOF Corporation (see, e.g., the website: www.nofamerica.com/store/index.php?dispatch=products.view&product_id=771), TT3 (see, e.g., Li et al., Nano Lett (2015) 15(12):8099-8107), OF-Deg-Lin (see, e.g., Fenton et al., Angew Chem Int Ed Engl (2018) 57(41):13582-13586), ssPalmE (see, e.g., Akita et al., Journal of Controlled Release (2015) 200:97-105), or L319 (Maier et al., Molecular Therapy (2013) 21(8):1570-1578).

In some embodiments, the carrier is a lipid nanoparticle (LNP). In certain embodiments, the LNP comprises a cationic lipid, e.g. one or more cationic lipids described herein or known in the art. In certain embodiments, the LNP comprises an ionizable lipid, e.g. one or more ionizable lipids described herein or known in the art. In some embodiments, the carrier is poly-L-arginine. In some embodiments, the carrier is DOTAP.

### Activities of the Compositions of the Disclosure

In some embodiments of any of the immunomodulatory compositions described herein, the immunomodulatory composition selectively activates TLR7 in one or more cells contacted and/or transfected with the immunomodulatory composition.

In some embodiments, an immunomodulatory composition of the disclosure induces or increases the expression or secretion of one or more type I interferons (IFNs) by one or more cells contacted with the immunomodulatory composition. Type I IFNs are a large group of interferon proteins that regulate the immune system. Type I IFNs include, without limitation, IFN-α (alpha), IFN-β (beta), IFN-κ (kappa), IFN-δ (delta), IFN-ε (epsilon), IFN-τ (tau), IFN-ω (omega), and IFN-ζ (zeta, also known as limitin).

In some embodiments, an immunomodulatory composition of the disclosure induces or increases the expression or secretion of one or more type I IFNs (e.g., one or more of IFN-α, IFN-β, IFN-κ, IFN-δ, IFN-ε, IFN-τ, IFN-ω, or IFN-ζ) by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-α by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-β by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-κ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-δ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-F by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-τ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-ω by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-ζ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure increases activity of TLR8 in one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, an immunomodulatory composition of the disclosure increases expression or secretion of inflammatory cytokines such as TNFα by one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, an immunomodulatory composition of the disclosure induces or increases the expression or secretion of NFk-B-dependent cytokines by one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, the expression or secretion of type I IFNs, inflammatory cytokines, and/or NFk-B-dependent cytokines by one or more cells may be measured using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the expression or secretion of type I IFNs, inflammatory cytokines, and/or NFk-B-dependent cytokines by one or more cells may assessed based on the mRNA levels of the type I IFNs, inflammatory cytokines, and/or NFk-B-dependent cytokines, e.g., using qPCR, RNA-sequencing, microarray-based methods, or any other suitable method for measuring mRNA known in the art.

In some embodiments, an immunomodulatory composition of the disclosure modulates the activity of one or more B cells, e.g., one or more naïve B cells, activated B cells, and/or memory B cells. In some embodiments, an immunomodulatory composition of the disclosure increases or enhances activation of one or more B cells, e.g., assessed based on expression of B cell activation markers such as CD69, CD80, or CD86. In some embodiments, an immunomodulatory composition of the disclosure increases or enhances B cell proliferation. In some embodiments, an immunomodulatory composition of the disclosure increases or enhances class switch recombination. In some embodiments, an immunomodulatory composition of the disclosure increases or enhances antibody secretion. In some embodiments, an immunomodulatory composition of the disclosure increases or enhances B cell expression or secretion of cytokines. In some embodiments, B cell activation markers and/or cytokines may be measured using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the expression or secretion of B cell activation markers and/or cytokines by one or more B cells may assessed based on the mRNA levels of the markers or cytokines, e.g., using qPCR, RNA-sequencing, microarray-based methods, or any other suitable method for measuring mRNA known in the art.

In some embodiments, an immunomodulatory composition of the disclosure modulates the levels of one or more interferon-stimulated genes (ISGs). In some embodiments, the one or more ISGs include any ISG known in the art. In some embodiments, the one or more ISGs include any of the ISGs described in Shamith et al., Nucleic Acids Research (2009) 37, suppl_1; Schoggins and Rice, CurrOpin Virol (2011) 1(6):519-525; Forster et al., Nucleic Acids Research (2013) (database issue): D1040-D1046; and Liu et al., PNAS (2012) 109(11):4239-4244. Examples of ISGs that may be assessed include, without limitation, IFIT1, CXCL10, CXCL11, ISG15, CCL8, 2′5′OAS, APOBEC3G, APOBEC3A, PKR, ISG56, Mx2, MDA5, IFI44, IRF7, OASL1, ISG20 IFIT2, IFIT3, IFITM3, OAS2, OAS3, IFI16, IRF1, MX1, or IDO.

In some embodiments, the levels of one or more ISGs may assessed using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the levels of one or more ISGs may assessed based on the mRNA levels of the one or more ISGs, e.g., using qPCR, RNA-sequencing, microarray-based methods, or any other suitable method for measuring mRNA known in the art.

### In Vitro Uses

In some aspects, provided herein are methods for selectively activating TLR7 in one or more cells. In some embodiments, the one or more cells are human cells. In some embodiments, the one or more cells are human peripheral blood mononuclear cells. In some embodiments, the one or more cells are plasmacytoid dendritic cells.

In some embodiments, the methods comprise contacting and/or transfecting the one or more cells with an immunomodulatory composition of the disclosure. In some embodiments, the methods further comprise contacting the one or more cells with guanosine or a guanosine derivative. In some embodiments, the guanosine derivative is 2′3′ cyclic GMP, 7-thia-8-oxoguanosine, 7-deazaguanosine, 7-allyl-8-oxoguanosine, 7-deaza-dG, 9-hexyl-guanine, or any combination thereof.

In some embodiments, an immunomodulatory composition of the disclosure, comprising an RNA ligand of the disclosure and a carrier, such as any suitable carrier known in the art or described herein (e.g., in the “Carriers” section), may be introduced into one or more cells using any suitable method known in the art, such as direct injection, microinjection, electroporation, lipofection, biolistics, and the like. In some embodiments, the carrier is a lipid nanoparticle (LNP). In certain embodiments, the LNP comprises a cationic lipid. In certain embodiments, the LNP comprises an ionizable lipid. In some embodiments, the carrier is poly-L-arginine. In some embodiments, the carrier is DOTAP.

TLR7 Activation

In some embodiments, an immunomodulatory composition of the disclosure selectively activates TLR7 in one or more cells.

In some embodiments, an immunomodulatory composition of the disclosure induces or increases the expression or secretion of one or more type I interferons (IFNs) by one or more cells.

In some embodiments, an immunomodulatory composition of the disclosure induces or increases the expression or secretion of one or more type I IFNs (e.g., one or more of IFN-α, IFN-β, IFN-κ, IFN-δ, IFN-ε, IFN-τ, IFN-ω, or IFN-ζ) by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-α by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-β by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-κ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-δ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-F by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-τ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-ω by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure induces or increases the expression or secretion of IFN-ζ by one or more cells contacted and/or transfected with the immunomodulatory composition of the disclosure, for example, as compared to corresponding one or more cells that are not contacted and/or transfected with the immunomodulatory composition (e.g., as compared to corresponding one or more cells treated with a negative control or with cell culture medium only). In some embodiments, the induction or increase is of at least about any of 1%, 5%, 10%, 20%, 30%, 40%, 50%, 60%, 70%, 80%, 90%, 100%, 150%, 200%, 300%, 350%, 400%, 450%, 500%, 550%, 600%, 650%, 700%, 750%, 800%, 850%, 900%, 950%, 1000% or more. In some embodiments, the one or more cells are human cells. In some embodiments, the one or more cells are human peripheral blood mononuclear cells. In some embodiments, the one or more cells are plasmacytoid dendritic cells.

In some embodiments, an immunomodulatory composition of the disclosure increases activity of TLR8 in one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, an immunomodulatory composition of the disclosure increases expression or secretion of inflammatory cytokines such as TNFα by one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, an immunomodulatory composition of the disclosure induces or increases the expression or secretion of NFk-B-dependent cytokines by one or more cells contacted and/or transfected with the immunomodulatory composition of the disclosure by less than about 50%, less than about 40%, less than about 30%, less than about 20%, less than about 15%, less than about 10%, less than about 5%, less than about 2%, or less than about 1%, for example, as compared to corresponding one or more cells that are not contacted and/or transfected with the immunomodulatory composition (e.g., as compared to corresponding one or more cells treated with a negative control or with cell culture medium only). In some embodiments, the one or more cells are human cells. In some embodiments, the one or more cells are human peripheral blood mononuclear cells. In some embodiments, the one or more cells are monocytes.

In some embodiments, the expression or secretion of type I IFNs, inflammatory cytokines, and/or NFk-B-dependent cytokines by one or more cells may be measured using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the expression or secretion of type I IFNs, inflammatory cytokines, and/or NFk-B-dependent cytokines by one or more cells may assessed based on the mRNA levels of the type I IFNs, inflammatory cytokines, and/or NFk-B-dependent cytokines, e.g., using qPCR, RNA-sequencing, microarray-based methods, or any other suitable method for measuring mRNA known in the art.

In some embodiments, an immunomodulatory composition of the disclosure modulates the activity of one or more B cells, e.g., one or more naïve B cells, activated B cells, and/or memory B cells. In some embodiments, an immunomodulatory composition of the disclosure increases or enhances activation of one or more B cells, e.g., assessed based on expression of B cell activation markers such as CD69, CD80, or CD86. In some embodiments, an immunomodulatory composition of the disclosure increases or enhances B cell proliferation. In some embodiments, an immunomodulatory composition of the disclosure increases or enhances class switch recombination. In some embodiments, an immunomodulatory composition of the disclosure increases or enhances antibody secretion. In some embodiments, an immunomodulatory composition of the disclosure increases or enhances B cell expression or secretion of cytokines. In some embodiments, B cell activation markers and/or cytokines may be measured using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the expression or secretion of B cell activation markers and/or cytokines by one or more B cells may assessed based on the mRNA levels of the markers or cytokines, e.g., using qPCR, RNA-sequencing, microarray-based methods, or any other suitable method for measuring mRNA known in the art.

In some embodiments, an immunomodulatory composition of the disclosure modulates the levels of one or more interferon-stimulated genes (ISGs). In some embodiments, the one or more ISGs include any ISG known in the art. In some embodiments, the one or more ISGs include any of the ISGs described in Shamith et al., Nucleic Acids Research (2009) 37, suppl_1; Schoggins and Rice, CurrOpin Virol (2011) 1(6):519-525; Forster et al., Nucleic Acids Research (2013) (database issue): D1040-D1046; Liu et al., PNAS (2012) 109(11):4239-4244. Examples of ISGs that may be assessed include, without limitation, IFIT1, CXCL10, CXCL11, ISG15, CCL8, 2′5′OAS, APOBEC3G, APOBEC3A, PKR, ISG56, Mx2, MDA5, IFI44, IRF7, OASL1, ISG20 IFIT2, IFIT3, IFITM3, OAS2, OAS3, IFI16, IRF1, MX1, or IDO.

In some embodiments, the levels of one or more ISGs may assessed using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the levels of one or more ISGs may assessed based on the mRNA levels of the one or more ISGs, e.g., using qPCR, RNA-sequencing, microarray-based methods, or any other suitable method for measuring mRNA known in the art.

Pharmaceutical Compositions

In some aspects, providing herein are pharmaceutical compositions that comprise an immunomodulatory composition of the disclosure. Such pharmaceutical compositions may include one or more optional pharmaceutically acceptable excipients (Remington's Pharmaceutical Sciences 16th edition, Osol, A. Ed. (1980)), and may be in the form of lyophilized formulations or aqueous solutions.

Pharmaceutically acceptable excipients are generally nontoxic to recipients at the dosages and concentrations employed. Exemplary pharmaceutically acceptable excipients that may be used in the pharmaceutical compositions of the disclosure include one or more of buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or surfactants.

Exemplary pharmaceutically acceptable excipients that may be used in the pharmaceutical compositions of the disclosure further include one or more interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Active ingredients of the pharmaceutical compositions of the disclosure may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules), or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers, which matrices are in the form of shaped articles, e.g. films, or microcapsules.

In some embodiments, a pharmaceutical composition of the disclosure comprises guanosine or a guanosine derivative. In some embodiments, the guanosine derivative is 2′3′ cyclic GMP, 7-thia-8-oxoguanosine, 7-deazaguanosine, 7-allyl-8-oxoguanosine, 7-deaza-dG, 9-hexyl-guanine, or any combination thereof.

The formulations to be used for in vivo administration are generally sterile. Sterility may be readily accomplished, e.g., by filtration through sterile filtration membranes.

In some embodiments, the pharmaceutical compositions of the disclosure are vaccine compositions. Vaccine compositions refer to pharmaceutical compositions that, upon administration, induce an immune response, e.g., a cellular immune response, which recognizes and attacks a pathogen or a diseased cell such as a cancer cell. A vaccine may be used for the prevention or treatment of a disease. Vaccine compositions of the disclosure may include an RNA ligand of the disclosure, a pharmaceutically acceptable carrier, e.g., poly-L-arginine or DOTAP, and one or more of a pharmaceutically acceptable excipient, any additional agent, compound, molecule or ingredient described herein, and/or any suitable additional agent known in the art.

In some embodiments, the pharmaceutical compositions of the disclosure are adjuvant compositions. Adjuvant compositions refer to any substance or combination of substances that, upon administration, enhance an immune response, e.g., a cellular immune response, which recognizes and attacks a pathogen or a diseased cell such as a cancer cell. An adjuvant may be used for the prevention or treatment of a disease. Adjuvant compositions of the disclosure may include an RNA ligand of the disclosure, a pharmaceutically acceptable carrier, e.g., poly-L-arginine or DOTAP, and one or more of a pharmaceutically acceptable excipient, any additional agent, compound, molecule or ingredient described herein, and/or any suitable additional agent known in the art. In some embodiments, an immunomodulatory composition of the disclosure, or a pharmaceutical composition of the disclosure may be used as an adjuvant for another treatment, such as a vaccine, e.g., an anti-viral vaccine, an anti-bacterial vaccine, or an anti-cancer vaccine.

Additional Therapeutic Agents

In some embodiments, the pharmaceutical compositions of the disclosure may contain more than one active component as necessary for the particular indication being treated, preferably those with complementary activities that do not adversely affect each other. For example, in addition to an immunomodulatory composition of the disclosure (i.e., comprising an RNA ligand of the disclosure), it may be desirable to include in the pharmaceutical composition, an additional agent, such as another immunomodulatory composition, a nucleic acid, a protein or polypeptide (e.g., an antibody or fragments thereof), a vaccine or vaccine composition, an adjuvant, and/or one or more drugs, e.g., a chemotherapeutic agent, cytotoxic agent, cytokine, growth inhibitory agent, anti-hormonal agent, and/or cardioprotectant. In some embodiments, a pharmaceutical composition of the disclosure comprises the immunomodulatory composition of the disclosure and one or more of an immunostimulatory agent, an anti-viral agent, an antibiotic, an anti-fungal agent, an anti-parasitic agent, an anti-bacterial agent, an anti-tumor agent, a chemokine, a growth factor, an anti-angiogenic factor, a chemotherapeutic agent, an antibody, a gene-silencing agent, or a cytokine (e.g., a type I IFN such as IFN-α and/or IFN-β). Such molecules or agents are suitably present in combination in amounts that are effective for the purpose intended.

Nucleic Acids, Vectors and Host Cells

In some embodiments, isolated nucleic acids having a nucleotide sequence comprising any of the RNA ligands of the present disclosure are provided. In some embodiments, one or more vectors (e.g., expression vectors) comprising such nucleic acids are provided. In some embodiments, a host cell comprising such nucleic acids or vectors is also provided. In some embodiments, the host cell is eukaryotic or prokaryotic. Host cells of the present disclosure also include, without limitation, isolated cells, in vitro cultured cells, and ex vivo cultured cells.

In some embodiments, methods of making an RNA ligand of the present disclosure are provided. In some embodiments, the method includes culturing a host cell of the present disclosure comprising a nucleic acid or vector comprising the RNA ligand, under conditions suitable for expression of the RNA ligand. In some embodiments, the RNA ligand is subsequently recovered from the host cell (or host cell culture medium). For recombinant production of an RNA ligand of the present disclosure, a nucleic acid comprising the RNA ligand is isolated and inserted into one or more vectors for further cloning and/or expression in a host cell. Such nucleic acid may be readily isolated and sequenced using conventional procedures.

Suitable vectors comprising a nucleic acid sequence comprising an RNA ligand of the present disclosure include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript {e.g, pBS SK+) and its derivatives, mpl8, mpl9, pBR322, pMB9, ColEl, pCRl, RP4, phage DNAs, and shuttle vectors such as pSA3 and pAT28. These and many other cloning vectors are available from commercial vendors such as BioRad, Strategene, and Invitrogen.

Suitable host cells for cloning or expression of an RNA ligand, nucleic acid or vector of the disclosure include prokaryotic or eukaryotic cells. For example, eukaryotic microorganisms, such as filamentous fungi or yeast may be used. Cells from plants and insects may also be used as host cells. Numerous baculoviral strains have been identified which may be used in conjunction with insect cells. Plant cell cultures can also be utilized as hosts. Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N. Y. Acad. Sci. 383:44-68 (1982); MRC 5 cells; and FS4 cells. Other useful mammalian host cell lines include Chinese hamster ovary (CHO) cells and myeloma cell lines.

### Therapeutic Methods

Provided herein are in vivo therapeutic uses of the immunomodulatory compositions and/or pharmaceutical compositions of the disclosure.

In some aspects, provided herein are methods for selectively activating TLR7 in an individual using the immunomodulatory compositions and/or pharmaceutical compositions of the disclosure. In some embodiments, the methods comprise administering to the individual a therapeutically effective amount of an immunomodulatory composition and/or pharmaceutical compositions of the disclosure. In some embodiments, the immunomodulatory compositions and/or pharmaceutical compositions of the disclosure are administered as single agents, or in combination with one or more additional therapeutic agents, e.g., as described herein. The methods for selectively activating TLR7 in an individual provided herein may find use in the prevention or treatment of certain diseases, disorders, or conditions such as infections, immune disorders, cancers; and/or in the modulation of the immune system in an individual.

As used herein, “preventing,” “prevention,” “prevent,” and the like, include providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition. An individual “at risk” of developing a disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, “treat,” “treating,” “treatment,” and the like, refer to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

As used herein, a “therapeutically effective amount” is at least the minimum amount of an agent, such as an immunomodulatory composition or pharmaceutical composition of the disclosure, required to effect a measurable improvement of a particular disease, disorder, or condition. A therapeutically effective amount herein may vary according to factors such as the disease state, age, sex, and weight of the patient, and the ability of the agent to elicit a desired response in the individual. A therapeutically effective amount is also one in which any toxic or detrimental effects of the agent are outweighed by the therapeutically beneficial effects.

### Infections

Toll-like receptors such as TLR7, as well as Type I IFNs have several known roles in regulating the innate and adaptive immune system responses to infections by pathogens such as viruses, bacteria, parasites, and fungi. See, e.g., Lester and Li, J Mol Biol (2014) 426(6):1246-1264; MacNab et al., Nat Rev Immunol (2020) 15(2):87-103. Accordingly, immunomodulatory compositions and/or pharmaceutical compositions of the disclosure that selectively activate TLR7 may find use in the prevention or treatment of viral infections, bacterial infections, parasitic infections, and/or fungal infections.

In some aspects, provided herein are methods for preventing or treating a viral infection in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the viral infection. Examples of viral infections that may be prevented or treated using the immunomodulatory compositions and/or pharmaceutical compositions of the disclosure include, without limitation, respiratory viruses such as adenoviruses, influenza viruses, parainfluenza viruses, respiratory syncytial viruses (RSV), coronaviruses, and rhinoviruses; mumps, measles, and other childhood viruses, such as paramyxoviruses, rubella virus, rubeola, parvoviruses, and varicella; poxviruses such as smallpox (variola); enteroviruses such as poliovirus, coxsackieviruses A and B, echovirus, echoviruses, and hepatitis A; hepatitis viruses, such as hepatitis A, B, C, D, and E; herpes viruses, such as human herpes simplex virus 1 (HHSV1, HSV-1), human herpes simplex virus 2 (HHSV2, HSV-2), human cytomegalovirus (HCMV), Epstein-Barr virus (EBV), varicella-zoster virus (VZV), human herpesvirus 6 (HHV6), Human herpesvirus 7 (HHSV7, HHV7), and human herpesvirus 8 (HHSV8, HHV8); rhabdoviruses such as lyssaviruses; papovaviruses such as human papillomaviruses (HPVs), and JC and BK viruses; retroviruses, such as HIV-1, HIV-2, and human T lymphocyte viruses (HTLV); arboviruses, such as dengue virus, yellow fever, Japanese encephalitis virus, West Nile virus, tick-borne encephalitis, vesiculovirus, Ross River virus, Syndvis virus, and Phlebovirus; prions; as well as other viral infections, including, without limitation, Ebola virus, Marburg virus, Lassa virus, African swine fever virus, Morbillivirus, Hantavirus, Lyssavirus, and severe acute respiratory syndrome (SARS).

In some aspects, provided herein are methods for preventing or treating a bacterial infection in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the bacterial infection. Examples of bacterial infections that may be prevented or treated using the immunomodulatory compositions and/or pharmaceutical compositions of the disclosure include, without limitation, Streptococcus agalactiae, Escherichia coli, Listeria monocytogenes, Streptococcus pneumonia, Haemophilus influenza, Neisseria meningitides, Klebsiella spp., Staphylococcus aureus, Streptococcus pneumonia, Mycobacterium tuberculosis, Cryptococcus neoformans, or Clostridium tetanus.

Other examples of bacterial infections that may be prevented or treated using the immunomodulatory compositions and/or pharmaceutical compositions of the disclosure include, without limitation, skin infections, such as Staphylococcus aureus, Streptococcus pyogenes, or Propionibacterium acne; ear infections, such as Streptococcus pneumonia, Moraxella catarrhalis, Haemophilus influenza, Pseudomonas aeruginosa, Staphylococcus aureus; eye infections, such as Staphylococcus aureus, Streptococcus pneumonia, Streptococcus pyogenes, Haemophilus influenza, enterococci, Enterobacteriaceae, Pseudomonas aeruginosa, Moraxella catarrhalis, Peptostreptococcus, Streptococcus viridans, Corynebacterium diphtheriae, Proteus mirabilis, Klebsiella pneumoniae, Serratia marcescens, Escherichia coli, Aeromonas hydrophila, Moraxella, Pasteurella multocida, Acinetobacter, Bacillus, Chlamydia trachomatis, Clostridium, Neisseria gonorrhoeae, Neisseria meningitides, or Staphylococcus epidermidis; respiratory tract infections, such as Streptococcus pneumonia, Staphylococcus aureus, Mycoplasma pneumonia, Chlamydophila pneumonia, Streptococcus pyogenes, Escherichia coli, Streptococcus agalactiae, Chlamydia trachomatis, Mycoplasma pneumonia, or Haemophilus influenza; or gastrointestinal infections, such as Streptococcus mutans, Streptococcus sobrinus, Streptococcus spp., Lactobacillus casei, Actinomyces spp., Fusobacterium spp., Prevotella intermedia, Treponema denticola, or Bifidobacterium spp.

In some aspects, provided herein are methods for preventing or treating a fungal infection in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the fungal infection. Examples of fungal infections that may be prevented or treated using the immunomodulatory compositions and/or pharmaceutical compositions of the disclosure include, without limitation, Absidia corymbifera, Acremoniumfalciforme, A. kiliense, A. recifei, Ajellomyces dermatitidis, A. capsulata, Aspergillus spp., (e.g., A. flavus, A. fumigatus, A. nidulans, A. niger, A. terreus), Candida spp. (e.g., C. albicans, C. glabrata, C. guillermondii, C. krusei, C. parapsilosis, C. ke yr, C. tropicalis), C. neoformans, Cunninghamella elegans, Emmonsia parva, Epidermophyton floccosum, Exophialia dermitidis, E. werneckii, E. jeanselmei, E. spinifera, E. richardsiae, Filobasidiella neoformans, Fonsecaea compacta, F. pedrosoi, Histoplasma capsulatum, Leptoshaeria senegarlensis, Madurella mycetomatis, M. grisea, Malassezia furfur, Microsporum spp, Neotestudina rosatii, Paracoccidioides brasiliensis, Penicillium marneffei, Phialophora verrucosa, Piedraia hortae, Pneumocystis carinii, Pseudallescheria boydii, Pyrenochaeta romeroi, Rhizomucor pusillus, Sporothrix schenckii, Trichophyton spp, Trichosporon beigelii, or Xylohypha bantiana.

In some aspects, provided herein are methods for preventing or treating a parasitic infection in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the parasitic infection. Examples of parasitic infections that may be prevented or treated using the immunomodulatory compositions and/or pharmaceutical compositions of the disclosure include, without limitation, worm infections, such as intestinal worm infections. Other examples of parasitic infections that may be prevented or treated using the immunomodulatory compositions and/or pharmaceutical compositions of the disclosure include, without limitation, Acanthamoeba, African Sleeping Sickness (African trypanosomiasis), alveolar echinococcosis (Echinococcosis, Hydatid Disease), amebiasis (Entamoeba histolytica), American trypanosomiasis (Chagas Disease), ancylostomiasis (Hookworm), angiostrongyliasis (Angiostrongylus), anisakiasis (Anisakis, Pseudoterranova), ascariasis (Ascaris, intestinal roundworms), babesiosis (Babesia), balantidiasis (Balantidium), balamuthia, Baylisascariasis (Baylisascaris, Raccoon Roundworm), bilharzia (Schistosomiasis), Blastocystis hominis infection, capillariasis (Capillaria), cercarial dermatitis (Swimmer's Itch), Chagas Disease (American trypanosomiasis), Chilomastix mesnili, clonorchiasis (Clonorchis), Cutaneous larva migrans, ancylostomiasis, cryptosporidiosis (Cryptosporidium), cyclosporiasis (Cyclospora), cysticercosis (Neurocysticercosis), cystoisospora (Cystoisosporiasis), isospora, Dientamoeba fragilis, diphyllobothriasis (Diphyllobothrium), dipylidium caninum infection, dirofilariasis (Dirofilaria), DPDx, dracunculiasis (Guinea Worm Disease), Dipylidium caninum, echinococcosis (Cystic, Alveolar Hydatid Disease), elephantiasis (Filariasis, Lymphatic Filariasis), Endolimax nana, entamoeba coli, entamoeba dispar, entamoeba hartmanni, entamoeba histolytica (Amebiasis), entamoeba polecki, enterobiasis (Pinworm), fascioliasis (Fasciola), fasciolopsiasis (Fasciolopsis), filariasis, giardiasis (Giardia), gnathostomiasis (Gnathostoma), heterophyiasis (Heterophyes), hydatid disease, hymenolepiasis (Hymenolepis), intestinal roundworms (Ascariasis, Ascaris infection), Iodamoeba buetschlii, Isospora, Kala-azar (Leishmaniasis, Leishmania), keratitis (Acanthamoeba), liver flukes (Clonorchiasis, Opisthorchiasis, Fascioliasis), loiasis (Loa loa), lymphatic filariasis, malaria (Plasmodium), microsporidiosis (Microsporidia), mites, lice, myiasis, naegleria, neurocysticercosis (Cysticercosis), ocular larva migrans (Toxocariasis, Toxocara, visceral larva migrans), onchocerciasis (River blindness), opisthorchiasis (Opisthorchis), paragonimiasis (Paragonimus), pediculosis, pthiriasis, Enterobiasis, Pneumocystis jirovecii pneumonia, Pseudoterranova (Anisakiasis, Anisakis), baylisascariasis (Baylisascaris), Sappinia, Sarcocystosis (Sarcocystosis), Schistosomiasis (Bilharzia), Sleeping Sickness (Trypanosomiasis), soil-transmitted helminths, Strongyloidiasis (Strongyloides), Taeniasis (Taenia), toxoplasmosis (Toxoplasma), trichinellosis (Trichinosis), trichinosis (Trichinellosis), trichomoniasis (Trichomonas), or trichuriasis (Whipworm, Trichuris).

### Immune Disorders

Toll-like receptors such as TLR7, as well as Type I IFNs have several known roles in the immunopathology of immune disorders, such as immunodeficiencies, autoimmune disorders or allergies. See, e.g., Uematsu and Akira, Expert Opin Biol Ther (2006) 6(3):203-14; Crow et al., Annual Review of Pathology: Mechanisms of Disease—Type I Interferons in Autoimmune Disease (2019) 14:369-393; and Wang et al., Front Immunol (2017) 8:1431. Accordingly, immunomodulatory compositions and/or pharmaceutical compositions of the disclosure that selectively activate TLR7 may find use in preventing or treating immune disorders, such as immunodeficiencies, autoimmune disorders or allergies.

In some aspects, provided herein are methods for preventing or treating an immune disorder in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the immune disorder. Examples of immune disorders that may be prevented or treated using the immunomodulatory compositions and/or pharmaceutical compositions of the disclosure include, without limitation, autoimmune diseases, immunodeficiencies, and allergies.

Non-limiting examples of allergies that may be prevented or treated using the immunomodulatory compositions and/or pharmaceutical compositions of the disclosure include seasonal allergies, mastocytosis, perennial allergies, anaphylaxis, food allergies, allergic rhinitis, or atopic dermatitis.

Non-limiting examples of immunodeficiencies that may be prevented or treated using the immunomodulatory compositions and/or pharmaceutical compositions of the disclosure include primary immunodeficiencies such as severe combined immunodeficiency (SCID), DiGeorge syndrome, hyperimmunoglobulin E syndrome (also known as Job's Syndrome), common variable immunodeficiency (CVID), chronic granulomatous disease (CGD), Wiskott-Aldrich syndrome (WAS), autoimmune lymphoproliferative syndrome (ALPS), hyper IgM syndrome, leukocyte adhesion deficiency (LAD), NF-κB Essential Modifier (NEMO) mutations, selective immunoglobulin A deficiency, X-linked agammaglobulinemia, X-linked lymphoproliferative disease (XLP), and ataxia-telangiectasia; secondary immunodeficiencies such as acquired immunodeficiency syndrome (AIDS); drug induced immunodeficiencies or immunosuppression; or immunosuppression caused by chronic hemodialysis, trauma, or surgical procedures.

Non-limiting examples of autoimmune disorders that may be prevented or treated using the immunomodulatory compositions and/or pharmaceutical compositions of the disclosure include lupus, scleroderma, hemolytic anemia, vasculitis, Type 1 diabetes, Graves' disease, arthritis (including rheumatoid arthritis, juvenile rheumatoid arthritis, osteoarthritis, psoriatic arthritis), multiple sclerosis, diabetes mellitus, Goodpasture syndrome, pernicious anemia, myopathy, Coeliac disease, inflammatory bowel disease, aplastic anemia, Lyme disease, encephalomyelitis, myasthenia gravis, systemic lupus erythematosis, autoimmune thyroiditis, dermatitis (including atopic dermatitis and eczematous dermatitis), psoriasis, Siogren's Syndrome, Crohn's disease, aphthous ulcer, iritis, conjunctivitis, keratoconjunctivitis, ulcerative colitis, asthma, allergic asthma, cutaneous lupus erythematosus, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, pure red cell anemia, idiopathic thrombocytopenia, polychondritis, Wegener's granulomatosis, chronic active hepatitis, Stevens-Johnson syndrome, idiopathic sprue, lichen planus, sarcoidosis, primary biliary cirrhosis, uveitis posterior, and interstitial lung fibrosis.

### Cancers

Toll-like receptors such as TLR7, as well as Type I IFNs have several known roles in regulating the anti-tumor immune response. See, e.g., Urban-Wojciuk et al., Front. Immunol (2019) 10:2388; Musella et al., (2017) 6(5):e1314424; and Zitvogel et al., Nature Reviews Immunology (2015) 15:405-414. Accordingly, immunomodulatory compositions and/or pharmaceutical compositions of the disclosure that selectively activate TLR7 may find use in preventing or treating cancers.

In some aspects, provided herein are methods for preventing or treating a cancer in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents for preventing or treating the cancer. Examples of cancers that may be prevented or treated using the immunomodulatory compositions and/or pharmaceutical compositions of the disclosure include, without limitation, carcinomas, sarcomas, lymphomas, leukemias, germ cell tumors, and blastomas. Specific examples of cancers that may be prevented or treated using the immunomodulatory compositions and/or pharmaceutical compositions of the disclosure include, without limitation, bone and muscle sarcomas, such as chondrosarcoma, Ewing's sarcoma, malignant fibrous histiocytoma of bone/osteosarcoma, osteosarcoma, rhabdomyosarcoma, and heart cancer; brain and nervous system cancers, such as astrocytoma, brainstem glioma, pilocytic astrocytoma, ependymoma, primitive neuroectodermal tumor, cerebellar astrocytoma, cerebral astrocytoma, glioblastoma, glioma, medulloblastoma, neuroblastoma, oligodendroglioma, pineal astrocytoma, pituitary adenoma, and visual pathway and hypothalamic glioma; breast cancers, such as invasive lobular carcinoma, tubular carcinoma, invasive cribriform carcinoma, medullary carcinoma, male breast cancer, phyllodes tumor, and inflammatory breast cancer; endocrine system cancers, such as adrenocortical carcinoma, islet cell carcinoma (endocrine pancreas), multiple endocrine, eoplasia syndrome, parathyroid cancer, pheochromocytoma, thyroid cancer, and Merkel cell carcinoma; eye cancers, such as uveal melanoma and retinoblastoma; gastrointestinal cancers, such as anal cancer, appendix cancer, cholangiocarcinoma, carcinoid tumor, gastrointestinal cancer, colon cancer extrahepatic bile duct cancer, gallbladder cancer, gastric (stomach) cancer, gastrointestinal carcinoid tumor, gastrointestinal stromal tumor (GIST), hepatocellular cancer, pancreatic cancer, islet cell cancer, and rectal cancer; genitourinary and gynecologic cancers, such as bladder cancer, cervical cancer, endometrial cancer, extragonadal germ cell tumor, ovarian cancer, ovarian epithelial cancer (surface epithelial-stromal tumor), ovarian germ cell tumor, penile cancer, renal cell carcinoma, renal pelvis and ureter, transitional cell cancer, prostate cancer, testicular cancer, gestational trophoblastic tumor, ureter and renal pelvis, transitional cell cancer, urethral cancer, uterine sarcoma, vaginal cancer, vulvar cancer, and Wilms tumor; head and neck cancers, such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers, such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B cell prolymphocytic leukemia, Burkitt's lymphoma, chronic lymphocytic leukemia, chronic myelogenous leukemia, cutaneous T-cell lymphoma, diffuse large B cell lymphoma, follicular lymphoma, hairy cell leukemia, hepatosplenic T-cell lymphoma, hodgkin's lymphoma, hairy cell leukemia, intravascular large B cell lymphoma, large granular lymphocytic leukemia, lymphoplasmacytic lymphoma, lymphomatoid granulomatosis, mantle cell lymphoma, marginal zone B cell lymphoma, mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sézary syndrome, splenic marginal zone lymphoma, and T-cell prolymphocytic leukemia; skin cancers, such as basal cell carcinoma, squamous cell carcinoma, skin adnexal tumors (e.g. sebaceous carcinoma), melanoma, merkel cell carcinoma, sarcomas of primary cutaneous origin (e.g., dermatofibrosarcoma protuberans), and lymphomas of primary cutaneous origin (e.g., mycosis fungoides); thoracic and respiratory cancers, such as bronchial adenomas/carcinoids, small cell lung cancer, mesothelioma, non-small cell lung cancer, pleuropulmonary blastoma, laryngeal cancer, thymoma, and thymic carcinoma; AIDS-related cancers; Kaposi sarcoma; epithelioid hemangioendothelioma (EHE); desmoplastic small round cell tumor and liposarcoma.

### Enhancement of Immune Responses

Toll-like receptors such as TLR7, as well as Type I IFNs have several known roles in regulating the immune system, including, but not limited to regulation of humoral immunity, adaptive immunity, and antigen-specific T-cell responses. See, e.g., Le Bon et al., Immunity (2001) 14(4):461-470; Fitzgerald and Kagan, Cell (2020) 180(6):1044-1066; and Li et al., Front Immunol (2019) 10:2191. Accordingly, immunomodulatory compositions and/or pharmaceutical compositions of the disclosure that selectively activate TLR7 may find use in increasing, inducing, or enhancing humoral immunity, adaptive immunity, B cell responses, and/or antigen-specific T-cell responses in an individual.

In some aspects, provided herein are methods for increasing, inducing, or enhancing humoral immunity in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing adaptive immunity in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing antigen-specific T-cell responses (e.g., CD8+ T cells) in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing B cell activation and/or proliferation in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing dendritic cell (e.g., cDC, plasmacytoid dendritic cell) activation and/or proliferation in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing IL-12 production in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing T cell priming in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing B cell class switch recombination in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing B cell antibody secretion in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some aspects, provided herein are methods for increasing, inducing, or enhancing B cell cytokine secretion in an individual. In some embodiments, the methods comprise administering to the individual a therapeutically effective amount of an immunomodulatory composition or pharmaceutical composition of the disclosure; or a therapeutically effective amount of an immunomodulatory composition or pharmaceutical composition of the disclosure in combination with one or more additional agents.

In some embodiments of any of the therapeutic methods described herein, the methods further comprise measuring the level of TLR7 activity in a sample obtained from the individual before and/or after the individual has received one or more doses of the immunomodulatory composition and/or pharmaceutical composition of the disclosure. In some embodiments, the methods further comprise measuring the level of one or more type I IFNs (e.g., one or more of IFN-α, IFN-β, IFN-κ, IFN-δ, IFN-ε, IFN-τ, IFN-ω, or IFN-ζ) in a sample obtained from the individual before and/or after the individual has received one or more doses of the immunomodulatory composition and/or pharmaceutical composition of the disclosure. In some embodiments, the methods further comprise measuring the level of IFN-α in a sample obtained from the individual before and/or after the individual has received one or more doses of the immunomodulatory composition and/or pharmaceutical composition of the disclosure. In some embodiments, the methods further comprise measuring the level of one or more interferon-stimulated genes (ISGs) in a sample obtained from the individual before and/or after the individual has received one or more doses of the immunomodulatory composition and/or pharmaceutical composition of the disclosure. In some embodiments, the one or more ISGs include any ISG known in the art, e.g., one or more of the ISGs described in Shamith et al., Nucleic Acids Research (2009) 37, suppl_1; Schoggins and Rice, CurrOpin Virol (2011) 1(6):519-525; Forster et al., Nucleic Acids Research (2013) (database issue): D1040-D1046; Liu et al., PNAS (2012) 109(11):4239-4244. Examples of ISGs that may be assessed include, without limitation, IFIT1, CXCL10, CXCL11, ISG15, CCL8, 2′5′OAS, APOBEC3G, APOBEC3A, PKR, ISG56, Mx2, MDA5, IFI44, IRF7, OASL1, ISG20 IFIT2, IFIT3, IFITM3, OAS2, OAS3, IFI16, IRF1, MX1, or IDO.

In some embodiments, the sample is a blood sample, a plasma sample, or a serum sample. In some embodiments, the sample is a tissue biopsy, e.g., from spleen, one or more lymph nodes, or from a tumor. In some embodiments, the levels of type I IFNs may be measured using any suitable method known in the art, such as enzyme-linked immunosorbent assay, immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the levels of type I IFNs may assessed based on the mRNA levels of the type I IFNs, e.g., using qPCR, RNA-sequencing, microarray-based methods, or any other suitable method for measuring mRNA known in the art. In some embodiments, the levels of type I IFNs may assessed by fluorescence in situ hybridization (FISH), qPCR or any other suitable method known in the art for measuring RNA levels in tissue samples. In some embodiments, the levels of one or more ISGs may assessed using any suitable method known in the art, such as enzyme-linked immunosorbent assay (ELISA), immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the levels of one or more ISGs may assessed based on the mRNA levels of the one or more ISGs, e.g., using qPCR, RNA-sequencing, microarray-based methods, or any other suitable method for measuring mRNA known in the art.

Dosages and Administration Dosages

Dosages and desired concentrations of immunomodulatory compositions and/or pharmaceutical compositions of the present disclosure may vary depending on the particular use envisioned, e.g., any of the therapeutic methods described herein. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the immunomodulatory compositions and/or pharmaceutical compositions of the present disclosure, normal dosage amounts may vary based on an individual's body weight and the route of administration. For repeated administrations over several days or longer, depending on the severity of the disease, disorder, or condition to be treated, the treatment is sustained until a desired suppression of symptoms is achieved.

Dosages for a particular immunomodulatory composition and/or pharmaceutical composition of the disclosure may be determined empirically in individuals who have been given one or more administrations of the immunomodulatory composition and/or pharmaceutical composition of the disclosure. In some embodiments, individuals are given incremental doses of an immunomodulatory composition or pharmaceutical composition of the disclosure.

In some embodiments, to assess efficacy of an immunomodulatory composition or pharmaceutical composition of the disclosure, a clinical symptom of any of the diseases, disorders, or conditions of the present disclosure can be monitored. In some embodiments, to assess efficacy of an immunomodulatory composition or pharmaceutical composition of the disclosure, the level of TLR7 activity may be measured in a sample obtained from the individual before and/or after the individual has received one or more doses of the immunomodulatory composition and/or pharmaceutical composition of the disclosure. In some embodiments, to assess efficacy of an immunomodulatory composition or pharmaceutical composition of the disclosure, the level of one or more type I IFNs (e.g., one or more of IFN-α, IFN-β, IFN-κ, IFN-δ, IFN-ε, IFN-τ, IFN-ω, or IFN-ζ) may be measured in a sample obtained from the individual before and/or after the individual has received one or more doses of the immunomodulatory composition and/or pharmaceutical composition of the disclosure. In some embodiments, to assess efficacy of an immunomodulatory composition or pharmaceutical composition of the disclosure, the level of IFN-α may be measured in a sample obtained from the individual before and/or after the individual has received one or more doses of the immunomodulatory composition and/or pharmaceutical composition of the disclosure. In some embodiments, to assess efficacy of an immunomodulatory composition or pharmaceutical composition of the disclosure, the level of one or more interferon-stimulated genes (ISGs) may be measured in a sample obtained from the individual before and/or after the individual has received one or more doses of the immunomodulatory composition and/or pharmaceutical composition of the disclosure. In some embodiments, the one or more ISGs include any ISG known in the art, e.g., one or more of the ISGs described in Shamith et al., Nucleic Acids Research (2009) 37, suppl_1; Schoggins and Rice, CurrOpin Virol (2011) 1(6):519-525; Forster et al., Nucleic Acids Research (2013) (database issue): D1040-D1046; Liu et al., PNAS (2012) 109(11):4239-4244. Examples of ISGs that may be assessed include, without limitation, IFIT1, CXCL10, CXCL11, ISG15, CCL8, 2′5′OAS, APOBEC3G, APOBEC3A, PKR, ISG56, Mx2, MDA5, IFI44, IRF7, OASL1, ISG20 IFIT2, IFIT3, IFITM3, OAS2, OAS3, IFI16, IRF1, MX1, or IDO.

In some embodiments, the sample is a blood sample, a plasma sample, or a serum sample. In some embodiments, the sample is a tissue biopsy, e.g., from spleen, one or more lymph nodes, or from a tumor. In some embodiments, the levels of type I IFNs may be measured using any suitable method known in the art, such as enzyme-linked immunosorbent assay, immunoblotting, immunoassays, such as a Luminex assay (see, e.g., www.rndsystems.com/what-luminex-assay), a bead-based immunoassay, electrochemiluminescence-based methods such as Meso Scale Discovery (MSD), intracellular staining of cytokines analyzed by flow cytometry, or mass spectrometry. In some embodiments, the levels of type I IFNs may assessed based on the mRNA levels of the type I IFNs, e.g., using qPCR, RNA-sequencing, microarray-based methods, or any other suitable method for measuring mRNA known in the art. In some embodiments, the levels of type I IFNs may assessed by fluorescence in situ hybridization (FISH), qPCR or any other suitable method known in the art for measuring RNA levels in tissue samples. In some embodiments, the levels of one or more ISGs may assessed based on the mRNA levels of the one or more ISGs, e.g., using qPCR, RNA-sequencing, microarray-based methods, or any other suitable method for measuring mRNA known in the art.

Administration of an immunomodulatory composition or pharmaceutical composition of the disclosure can be continuous or intermittent, depending, for example, on the recipient's physiological condition, whether the purpose of the administration is therapeutic or prophylactic, and other factors known to skilled practitioners. The administration of an immunomodulatory composition or pharmaceutical composition of the disclosure may be essentially continuous over a preselected period of time or may be in a series of spaced doses.

It is within the scope of the present disclosure that different formulations will be effective for different treatments and different diseases, disorders, or conditions, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

Administration

Immunomodulatory compositions and/or pharmaceutical compositions of the present disclosure containing an RNA ligand of the present disclosure may be administered to an individual in need of thereof, in accordance with known methods, such as intravenous administration as a bolus or by continuous infusion over a period of time, by intramuscular, intraperitoneal, intracerobrospinal, intracranial, intraspinal, subcutaneous, intra-articular, intrasynovial, intravenous, intraarterial, intrathecal, oral, topical, or inhalation routes. In some embodiments, an immunomodulatory composition and/or pharmaceutical composition of the present disclosure is administered by intravenous infusion. In some embodiments, an immunomodulatory composition and/or pharmaceutical composition of the present disclosure is administered by local injection, e.g., into a tissue or into an organ. Administration of an immunomodulatory composition or pharmaceutical composition of the present disclosure may be performed using any suitable method known in the art, such as using catheters, syringes, or like devices to deliver the immunomodulatory composition or pharmaceutical composition into a target organ or tissue.

Immunomodulatory compositions or pharmaceutical compositions of the present disclosure may be administered in any suitable form known in the art. For example, an immunomodulatory composition or pharmaceutical composition of the present disclosure may be formulated for administration as a liposome, a polymeric microparticle, or an emulsion.

In some embodiments, an immunomodulatory composition or a pharmaceutical composition of the present disclosure is formulated for administration as liposomes. Various amphiphilic lipids can form bilayers in an aqueous environment to encapsulate a RNA-ligand-containing aqueous core as a liposome. These lipids can have an anionic, cationic or zwitterionic hydrophilic head groups. Lipids that may be used include any lipid described herein, e.g., in the “Carriers” section, or know in the art. In some embodiments, liposomes of the disclosure include one or more phospholipids, such as, but not limited to, phosphatidylethanolamines, phosphatidylcholines, phosphatidylserines, and phosphatidylglycerols. In some embodiments, liposomes of the disclosure include one or more cationic lipids, such as, but not limited to, DOTAP, 1,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), DOTMA, or 1,2-dilinolenyloxy-N,N-dimethyl-3-aminopropane (DLenDMA). In some embodiments, liposomes of the disclosure include one or more Zwitterionic lipids, such as, but not limited to, acyl zwitterionic lipids, ether zwitterionic lipids, DPPC, DOPC or dodecylphosphocholine. In some embodiments, liposomes of the disclosure include saturated and/or unsaturated lipds. In some embodiments, liposomes of the disclosure include a single lipid or a mixture of lipids. In some embodiments, the mixture may comprise (i) a mixture of anionic lipids, (ii) a mixture of cationic lipids, (iii) a mixture of zwitterionic lipids, (iv) a mixture of anionic lipids and cationic lipids, (v) a mixture of anionic lipids and zwitterionic lipids, (vi) a mixture of zwitterionic lipids and cationic lipids, or (vii) a mixture of anionic lipids, cationic lipids and zwitterionic lipids. In some embodiments, liposomes of the disclosure include non-amphiphilic lipids, such as cholesterol. In some embodiments, lipids within liposomes of the disclosure may be modified by covalent attachment of a polyethylene glycol (i.e., PEGylated). See, e.g., Heyes et al. (2005) J Controlled Release 107:276-87. In some embodiments, liposomes of the disclosure are multilamellar vesicles (MLV), small unilamellar vesicles (SUV), large unilamellar vesicles (LUV), or mixtures thereof. MLVs have multiple bilayers in each vesicle, forming several separate aqueous compartments. SUVs and LUVs have a single bilayer encapsulating an aqueous core. SUVs typically have a diameter <50 nm, and LUVs have a diameter >50 nm. Any suitable method for the preparation of liposomes may be used, for example, as described in Weissing V (ed.). Liposomes: Methods and Protocols, Vol. 1, Springer, 12, 29-50 (2010); and Functional Polymer Colloids and Microparticles volume 4 (Microspheres, microcapsules & liposomes) (eds. Arshady & Guyot). Citus Books, 2002.

In some embodiments, an immunomodulatory composition or a pharmaceutical composition of the present disclosure is formulated for administration as polymeric microparticles. Various polymers can form microparticles to encapsulate or adsorb RNA. Polymers that may be used include any polymer or polymeric molecule or agent described herein, e.g., in the “Carriers” section, or know in the art. Examples of polymers that may be used in the microparticles of the disclosure include, without limitation, poly(alpha-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones, polyester-amides, polyamino acids (e.g., poly-L-arginine, poly-L-lysine, poly-L-ornithine), and combinations thereof. Techniques for preparing suitable microparticles are well known in the art. To facilitate adsorption of RNA, a microparticle may include a cationic surfactant and/or lipid. An alternative way of making polymeric microparticles is by molding and curing e.g., as disclosed in WO2009/132206.

In some embodiments, an immunomodulatory composition or a pharmaceutical composition of the present disclosure is formulated for administration as an emulsion. Emulsions of the disclosure may comprise one or more oils, such as, without limitation, oils from an animal, vegetable or synthetic source. The emulsion may comprise a combination of oils. The aqueous component of the emulsion may be water, e.g., water for injection, and may include further components e.g., solutes. For instance, it may include salts to form a buffer e.g., citrate or phosphate salts, such as sodium salts. Typical buffers include a phosphate buffer; a Tris buffer; a borate buffer; a succinate buffer; a histidine buffer; or a citrate buffer. In some embodiments, the emulsion comprises a cationic lipid, such as any cationic lipid described herein or known in the art. In some embodiments, the emulsion comprises a non-ionic surfactant and/or a zwitterionic surfactant. Such surfactants include, but are not limited to the polyoxyethylene sorbitan ester surfactants (commonly referred to as the Tweens), e.g., polysorbate 20 and polysorbate 80; copolymers of ethylene oxide (EO), propylene oxide (PO), and/or butylene oxide (BO), such as linear EO/PO block copolymers; octoxynols, such as octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the Spans), such as sorbitan trioleate (Span 85) and sorbitan monolaurate. In some embodiments, the emulsion comprises mixtures of any suitable surfactant described herein and/or known in the art. The absolute amounts of oil and surfactant, and their ratio, can be varied within wide limits while still forming an emulsion. A skilled person can vary the relative proportions of the components to obtain a desired emulsion. Droplet size may be assessed using any method known in the art, such as dynamic light scattering and/or single-particle optical sensing e.g., the Accusizer™ and Nicomp™ series of instruments available from Particle Sizing Systems (Santa Barbara, USA), or the Zetasizer™ instruments from Malvern Instruments (UK), or the Particle Size Distribution Analyzer instruments from Horiba (Kyoto, Japan). Emulsions of the disclosure may be prepared using any suitable method known in the art, such as microfluidisation or thermal methods.

Administration of an immunomodulatory composition and/or a pharmaceutical composition of the disclosure may be performed in combination or in conjunction with another compound, composition, or agent. Such administration includes simultaneous administration and/or administration at different times. Administration in conjunction or in combination also encompasses administration as a co-formulation or administration as separate compositions, including at different dosing frequencies or intervals, and using the same route of administration or different routes of administration.

### Kits

The present disclosure also provides kits comprising an immunomodulatory composition, a nucleic acid, a vector, a pharmaceutical composition, or a vaccine composition of the present disclosure. Kits of the present disclosure may include one or more containers comprising an immunomodulatory composition, a nucleic acid, a vector, a pharmaceutical composition, or a vaccine composition of the present disclosure. In some embodiments, the kits further include instructions for use in accordance with the methods of this disclosure. In some embodiments, these instructions comprise a description of administration or use of the immunomodulatory composition, nucleic acid, vector, pharmaceutical composition, or vaccine composition of the present disclosure to prevent, reduce risk, or treat an individual having a disease, disorder, or condition described herein according to any methods of this disclosure. The instructions generally include information as to dosage, dosing schedule, and route of administration for the intended treatment.

In some embodiments, the kit may further include an additional agent (e.g., in the same or in one or more additional containers), such as another immunomodulatory composition, a nucleic acid, a protein or polypeptide (e.g., an antibody or fragments thereof), a vaccine or vaccine composition, an adjuvant, and/or one or more drugs, e.g., a chemotherapeutic agent, cytotoxic agent, cytokine, growth inhibitory agent, anti-hormonal agent, and/or cardioprotectant. In some embodiments, the kit may further include (e.g., in the same or in one or more additional containers) one or more of an immunostimulatory agent, an anti-viral agent, an antibiotic, an anti-fungal agent, an anti-parasitic agent, an anti-bacterial agent, an anti-tumor agent, a chemokine, a growth factor, an anti-angiogenic factor, a chemotherapeutic agent, an antibody, a gene-silencing agent, or a cytokine (e.g., a type I IFN such as IFN-α and/or IFN-β). In some embodiments, the kit may further include instructions for using the immunomodulatory composition, nucleic acid, vector, pharmaceutical composition, or vaccine composition of the present disclosure in combination with an additional agent, such as another immunomodulatory composition, a nucleic acid, a protein or polypeptide (e.g., an antibody or fragments thereof), a vaccine or vaccine composition, an adjuvant, one or more drugs, e.g., a chemotherapeutic agent, cytotoxic agent, cytokine, growth inhibitory agent, anti-hormonal agent, cardioprotectant, an immunostimulatory agent, an anti-viral agent, an antibiotic, an anti-fungal agent, an anti-parasitic agent, an anti-bacterial agent, an anti-tumor agent, a chemokine, a growth factor, an anti-angiogenic factor, a chemotherapeutic agent, an antibody, a gene-silencing agent, or a cytokine (e.g., a type I IFN such as IFN-α and/or IFN-β), according to any methods of this disclosure. The instructions generally include information as to dosage, dosing schedule, and route of administration for the intended treatment.

The containers in the kits may be unit doses, bulk packages (e.g., multi-dose packages) or sub-unit doses.

Instructions supplied in the kits of the present disclosure are typically written instructions on a label or package insert (e.g., a paper sheet included in the kit), but machine-readable instructions (e.g., instructions carried on a magnetic or optical storage disk) are also included in the disclosure. The label or package insert indicates that the immunomodulatory composition, nucleic acid, vector, pharmaceutical composition, or vaccine composition, and any additional agents, are used for treating or preventing, e.g., a disease, disorder, or condition of the present disclosure. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle).

Kits may optionally provide additional components such as buffers and interpretive information. Normally, the kit comprises a container and a label or package insert(s) on or associated with the container.

All citations throughout the disclosure are hereby expressly incorporated by reference.

### EXAMPLES

The present disclosure will be more fully understood by reference to the following Examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

In the nucleotide sequences shown in the Examples below, an asterisk (*) indicates a phosphorothioate linkage and a “-” indicates a phosphodiester linkage.

### Example 1: Design of TLR7-Selective RNA Ligands

This Example describes the design of TLR7-selective ligands with RNA modifications that block RNase cleavage at certain nucleotides to avoid the generation of uridine monomers that have the potential to activate TLR8.

A. Selection of Initial RNA Sequences to Generate TLR7-Selective Ligands with RNA Modifications that Block RNase Cleavage

To achieve maximum TLR7 selectivity, RNA sequences which were known to have some selectivity for TLR7 over TLR8 were selected to generate TLR7-selective ligands.

### ORN7013

The first selected RNA sequence, termed oligoribonucleotide 7013 (ORN7013), has the nucleotide sequence C*C*G*A*G*C*C*G*C*U*U*U*U*C*C*C (SEQ ID NO: 1) and was derived from Forsbach et al., J Immunology (2008) 180:3729-3738. ORN7013 contains four uridines flanked by non-stimulatory sequences and is fully phosphorothioate-modified.

### ORN7023

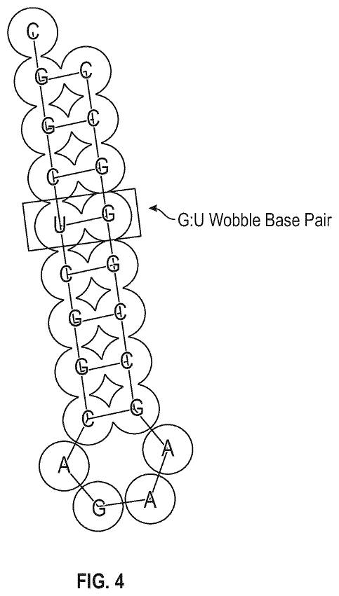

The second selected RNA sequence was derived from R2153, as described by Hartmann et al in WO2010/105819, and is referred to herein as ORN7023. ORN7023 has the nucleotide sequence C-G-G-C-U-C-G-G-C-A-G-A-A-G-C-C-G-G-G-C-C (SEQ ID NO: 2) and forms a short canonical RNA hairpin by pairing of the underlined sequences, including a G:U wobble base pair (FIG. 4).

### ORN7005 & ORN7009

Additional selected RNA sequences included ORN7005, which has the nucleotide sequence U-C-U-C-U-C-U-C-U-C-U-C-U-C-U-C-U-C-U-C(SEQ ID NO: 3), and ORN7009, which has the nucleotide sequence C-C-U-U-C-U-U-C-U-U-C-U-U-C-U-U-C-U-U-C(SEQ ID NO: 4). ORN7005 and ORN7009 were derived from pU1C and pU2C, respectively (see, e.g., Zhang et al., 2018, Cell Reports 25, 3371-3381). ORN7005 and ORN7009 are based on a phosphodiester (PDE) backbone. ORN7005 and ORN7009 do not contain the UG motif that is the most active at the TLR8 Binding Site #2 (see, e.g., Tanji et al Nat Struct Mol Biol. 2015 eb; 22(2):109-15), and the addition of guanosine as a separate agent is likely necessary to achieve TLR7 activation.

### ORN7001

Finally, an additional ligand was designed de novo with a tandem sequence containing a guanosine flanked by adenosines to serve as a guanosine source for TLR7 Binding Site #1, and to avoid TLR8 binding of Binding Site #2. This de novo-designed ligand, termed ORN7001, has the nucleotide sequence C-A-G-A-C-C-U-U-C-C-A-G-A-C-C-U-U-C (SEQ ID NO: 5). The sequence of ORN7001 also contains a C/U-rich C-U-U-C motif with two potential motifs for TLR7 Binding Site #2: C-U-U and U-U-C.

A summary of the initial RNA sequences selected to generate TLR7-selective ligands is provided in Table 1.

TABLE 1
Selected RNA sequences for generation of TLR7-selective ligands.

| Sequence Description | Sequence | SEQ ID NO |
|---|---|---|
| ORN7013 (7013_TLR7_AF_pto) | C*C*G*A*G*C*C*G*C*U* | 1 |
| (Sequence derived from Forsbach et al., J | U*U*U*C*C*C | |
| Immunology (2008) 180:3729-3738) | | |
| Underlined is a motif that can be degraded | | |
| into TLR7 and TLR8 ligands. The | | |
| underlined sequence corresponds to SEQ ID | | |
| NO: 7. | | |
| ORN7023 (7023u_TLR7_GH_R2153) | C-G-G-C-U-C-G-G-C-A-G-A- | 2 |
| (Sequence derived from WO2010/105819) | A-G-C-C-G-G-G-C-C | |
| Underlined is a TLR7 Binding Site #2 | | |
| motif. The underlined sequence corresponds | | |
| to SEQ ID NO: 21. | | |
| ORN7005 (7005_TLR7_ZZ_pU1C) | U-C-U-C-U-C-U-C-U-C-U-C- | 3 |
| (Sequence derived from pU1C of Zhang et | U-C-U-C-U-C-U-C | |
| al., 2018, Cell Reports 25, 3371-3381) | | |
| ORN7009 (7009_TLR7_ZZ_pU2C) | C-C-U-U-C-U-U-C-U-U-C-U- | 4 |
| (Sequence derived from pU2C of Zhang et | U-C-U-U-C-U-U-C | |
| al., 2018, Cell Reports 25, 3371-3381) | | |
| ORN7001 (7001_TLR7_AH) | C-A-G-A-C-C-U-U-C-C-A-G- | 5 |
| (de novo designed sequence) | A-C-C-U-U-C | |
| Underlined are C/U-rich C-U-U-C motifs | | |
| with two potential motifs for TLR7 Binding | | |
| Site #2: C-U-U and U-U-C. | | |

The asterisk (*) indicates a phosphorothioate (pto) modification between the indicated nucleosides; “-” indicates phosphodiester linkages between the indicated nucleosides.

B. Selection of Positions within the Selected RNA Sequences Suitable for Backbone Modification to Generate TLR7-Selective Ligands

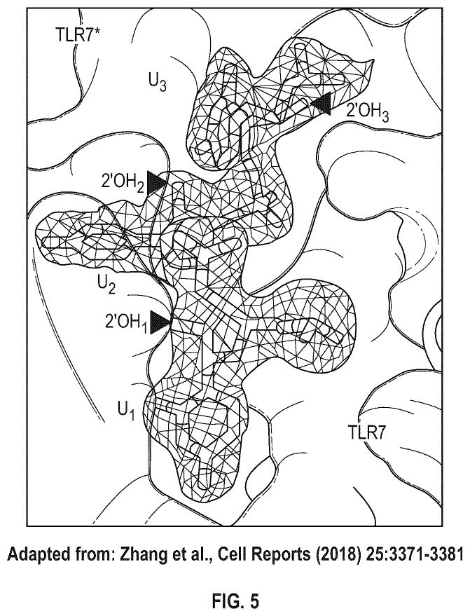

As shown in FIG. 5, the uridine in the center of the RNA trimer (U₁-U₂-U₃) bound by TLR7 at Binding Site #2 is deeply embedded in the binding pocket, including the 2′OH (2′OH₂). Suggesting that modification of this 2′OH could affect TLR7 activity, and is therefore likely not available for modification. In contrast, the 2′OH moieties of the flanking nucleotides (2′OH₁ and 2′OH₃) appear to be located to more open parts of the binding pocket (FIG. 5). Therefore, modification of the nucleotides directly 5′ of all uridines that could become uridine monomers was pursued.

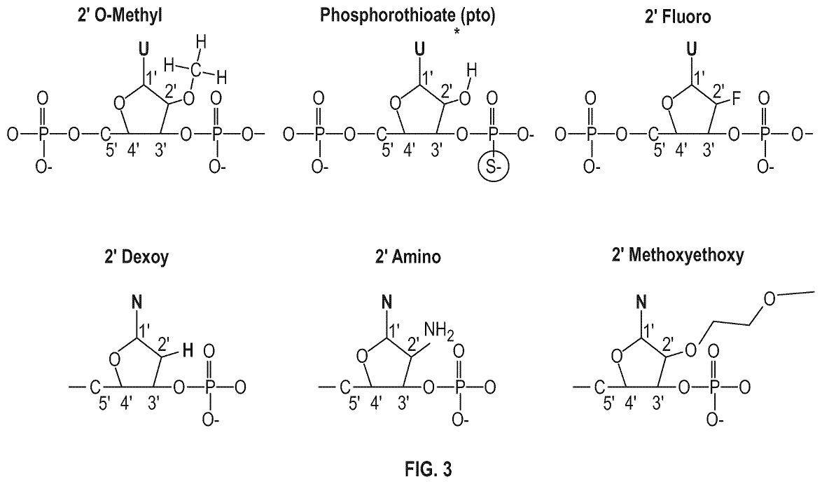

Both the less bulky, but less nuclease-resistant 2′-fluoro modification, and the nuclease resistant, but more bulky 2′-O-methyl modification were pursued to achieve RNase resistance. The 2′-fluoro modification deprives the RNA backbone of the 2′OH required for RNase activity, but can still be targeted by exonucleases. In contrast, 2′-O-methylation introduces a bulky modification that blocks binding of any nucleases. See, e.g., FIG. 3.

### C. Introduction of RNA Modifications to Generate TLR7-Selective Ligands

RNA modifications that specifically block RNase-mediated cleavage at certain nucleotides were introduced into the selected RNA sequences described above. The aim of the modifications was to enable the generation of RNA trimer degradation products that bind to TLR7 Binding Site #2 (i.e., having the sequence N-U-N, N*U*N, or N-U*N wherein N represents any ribonucleotide), and that cannot be degraded to monomeric U, which is a ligand for TLR8 Binding Site #1.

ORN7013 Modifications

As shown below, the nucleotide sequence of ORN7013 (SEQ ID NO: 1), derived from Forsbach et al., J Immunology (2008) 180:3729-3738, contains the underlined motif C*U*U*U*U*C, which can activate TLR7 and TLR8:

(SEQ ID NO: 1)
C*C*G*A*G*C*C*G*C*U*U*U*U*C*C*C

The C*U*U*U*U*C motif can be degraded to the TLR7 Binding Site #2 motifs C*U*U, U*U*U, or U*U*C, and can be further degraded to monomeric U, which is a TLR8 Binding Site #1 ligand.

To generate a TLR7-selective ligand, U₂ and U₄ of the C*U*U*U*U*C motif were modified. This allowed preservation of the central U within the TLR7 Binding Site #2 motif as a regular (i.e., unmodified) ribonucleotide (see, e.g., FIG. 5). Modification of U₂ and U₄ of the C*U*U*U*U*C motif resulted in the linkage between U₂ and U₃, as well as U₄ and U₅, being protected from RNase degradation, thereby preventing the generation of monomeric U. This protection from RNase degradation also allowed exchange of the phosphorothioate linkage between U₂ and U₃, as well as between U₄ and U₅, for a phosphodiester linkage. This reduced modification, particularly of the already modified space between the two respective nucleotides.

The modifications of the C*U*U*U*U*C motif described above led to the new motif C*X-U*X-U*C, with X representing either 2′-Fluoro-U or 2′O-methyl-U. Assuming that the unmodified U is the central U of the TLR7 Binding Site #2 motif, the remaining motifs would be X₂-U₃*X₄, and X₄-U₅*C₆. As shown in Table 2, the combination of the two modified sites with two different modifications led to four ORNs with individual motifs, termed ORN7014-7017 (corresponding to SEQ ID NOs: 12-15).

TABLE 2
Modifications of ORN7013 to generate a TLR7-selective ligand.

| Sequence Description | Sequence | SEQ ID NO |
|---|---|---|
| ORN7014 | C*C*G*A*G*C*C*G*C*mU-U*mU- | 12 |
| (7014_TLR7_AF_pto_2ome) | U*C*C*C | |
| The underlined C*mU- | | |
| U*mU-U*C motif | | |
| corresponds to SEQ ID NO: | | |
| 44, and is a 2′O-methyl- | | |
| modified version of the | | |
| TLR7/TLR8 motif in | | |
| ORN7013 (corresponding to | | |
| SEQ ID NO: 7). | | |
| ORN7015 | C*C*G*A*G*C*C*G*C*fU-U*fU- | 13 |
| (7015_TLR7_AF_pto_2F) | U*C*C*C | |
| The underlined C*fU-U*fU- | | |
| U*C motif corresponds to | | |
| SEQ ID NO: 45, and is a 2′- | | |
| fluoro modified version of | | |
| the TLR7/TLR8 motif in | | |
| ORN7013 (corresponding to | | |
| SEQ ID NO: 7). | | |
| ORN7016 | C*C*G*A*G*C*C*G*C*mU-U*fU- | 14 |
| (7016_TLR7_AF_pto_omeF) | U*C*C*C | |
| The underlined C*mU- | | |
| U*fU-U*C motif | | |
| corresponds to SEQ ID NO: | | |
| 46, and is a 2′-fluoro- and | | |
| 2′O-methyl-modified version | | |
| of the TLR7/TLR8 motif in | | |
| ORN7013 (corresponding to | | |
| SEQ ID NO: 7). | | |
| ORN7017 | C*C*G*A*G*C*C*G*C*fU-U*mU- | 15 |
| (7017_TLR7_AF_pto_Fome) | U*C*C*C | |
| The underlined C*fU- | | |
| U*mU-U*C motif | | |
| corresponds to SEQ ID NO: | | |
| 47, and is a 2′-fluoro- and | | |
| 2′O-methyl-modified version | | |
| of the TLR7/TLR8 motif in | | |
| ORN7013 (corresponding to | | |
| SEQ ID NO: 7). | | |

The asterisk (*) indicates a phosphorothioate (pto) modification between the indicated nucleosides; “m” indicates a 2′O-methyl modification; “f” indicates a 2′-fluoro modification; “-” indicates phosphodiester linkages between the indicated nucleosides.

ORN7013, derived from Forsbach et al., J Immunology (2008) 180:3729-3738, was also modified to reduce phosphorothioate modifications. Specifically, all nucleotides, except for nucleotides in the C*U*U*U*U*C motif, were linked by a phosphodiester backbone instead of phosphorothioate-modified backbone. This resulted in ORN7022, which has the nucleotide sequence C-C-G-A-G-C-C-G-C*U*U*U*U*C-C-C(SEQ ID NO: 16). Modified ORNs equivalent to ORN7014-7017 were designed based on ORN7022, resulting in ORN7018-ORN7021, corresponding to SEQ ID NOs: 17-20 (Table 3).

TABLE 3
Additional modifications of ORN7013 with
reduced phosphorothioate linkages to
generate a TLR7-selective ligand.

| Sequence Description | Sequence | SEQ ID NO |
|---|---|---|
| ORN7022 | C-C-G-A-G-C-C-G-C* | 16 |
| (7022_TLR7_AF_pto_Uonly) | U*U*U*U*C-C-C | |
| ORN7022 is equivalent to | | |
| ORN7013, except that all | | |
| nucleotides, except for | | |
| nucleotides in the underlined | | |
| C*U*U*U*U*C motif, are | | |
| linked by a phosphodiester | | |
| backbone. | | |
| ORN7018 | C-C-G-A-G-C-C-G-C- | 17 |
| (7018_TLR7_AF_2ome) | mU-U*mU-U*C-C-C | |
| The underlined C-mU- | | |
| U*mU-U*C motif | | |
| corresponds to SEQ ID NO: | | |
| 48, and is a 2′O-methyl- | | |
| modified version of the | | |
| TLR7/TLR8 motif in | | |
| ORN7013 (corresponding to | | |
| SEQ ID NO: 7). | | |
| ORN7019 | C-C-G-A-G-C-C-G-C- | 18 |
| (7019_TLR7_AF_2F) | fU-U*fU-U*C-C-C | |
| The underlined C-fU-U*fU- | | |
| U*C motif corresponds to | | |
| SEQ ID NO: 49, and is a 2′- | | |
| fluoro-modified version of | | |
| the TLR7/TLR8 motif in | | |
| ORN7013 (corresponding to | | |
| SEQ ID NO: 7). | | |
| ORN7020 | C-C-G-A-G-C-C-G-C- | 19 |
| (7020_TLR7_AF_omeF) | mU-U*fU-U*C-C-C | |
| The underlined C-mU-U*fU- | | |
| U*C motif corresponds to | | |
| SEQ ID NO: 50, and is a 2′- | | |
| fluoro- and 2′O-methyl- | | |
| modified version of the | | |
| TLR7/TLR8 motif in | | |
| ORN7013 (corresponding to | | |
| SEQ ID NO: 7). | | |
| ORN7021 | C-C-G-A-G-C-C-G-C- | 20 |
| (7021_TLR7_AF_Fome) | fU-U*mU-U*C-C-C | |
| The underlined C-fU-U*mU- | | |
| U*C motif corresponds to | | |
| SEQ ID NO: 51, and is a 2′- | | |
| fluoro- and 2′O-methyl- | | |
| modified version of the | | |
| TLR7/TLR8 motif in | | |
| ORN7013 (corresponding to | | |
| SEQ ID NO: 7). | | |

The asterisk (*) indicates a phosphorothioate (pto) modification between the indicated nucleosides; “m” indicates a 2′O-methyl modification; “f” indicates a 2′-fluoro modification; “-” indicates phosphodiester linkages between the indicated nucleosides.

ORN7023 Modifications

As shown below, the nucleotide sequence of ORN7023 (SEQ ID NO: 2), derived from WO2010/105819 (Hartmann et al), contains only one TLR7 Binding Site #2 motif (underlined), which is composed of nucleotides 4-6 of ORN7023: C-U-C:

(SEQ ID NO: 2)
C-G-G-C-U-C-G-G-C-A-G-A-A-G-C-C-G-G-G-C-C

Since it was assumed that the central U₂ in the C₁-U₂-C₃ motif would be required to remain unmodified (see, e.g., FIG. 5), C₁ within the motif was modified with 2′fluoro- or 2′O-methyl modifications, leading to the modified motif: D-U-C, with D representing either 2′-Fluoro-C or 2′O-methyl-C. This resulted in the 2′-fluoro-C-modified ORN7023F (SEQ ID NO: 23), with the motif fC-U-C; and the 2′G-methyl-C-modified ORN7-230me (SEQ ID NO: 24), with the motif mC-U-C.

An additional version of ORN7023 was generated, containing a 2′ unmodified sequence and the backbone converted from phosphodiester to phosphorothioate linkages (ORN7024; SEQ ID NO: 25).

The modified ORNs derived from ORN7023 are summarized in Table 4.

TABLE 4
Modifications of ORN7023 to generate
a TLR7-selective ligand.

| | | SEQ |
|---|---|---|
| | | ID |
| Sequence Description | Sequence | NO |
| ORN7023F | C-G-G-fC-U-C- | 23 |
| (7023F_TLR7_GH_R2153_F) | G-G-C-A-G-A- | |
| The underlined fC-U-C motif | A-G-C-C-G-G- | |
| corresponds to SEQ ID NO: 42, | G-C-C | |
| and is a 2′-fluoro-modified | | |
| version of the TLR7 Binding | | |
| Site #2 motif from ORN7023. | | |
| ORN7023Ome | C-G-G-mC-U-C- | 24 |
| (7023Ome_TLR7_GH_R2153 | G-G-C-A-G-A- | |
| ome) | A-G-C-C-G-G- | |
| The underlined mC-U-C motif | G-C-C | |
| corresponds to SEQ ID NO: 43, | | |
| and is a 2′O-methyl-modified | | |
| version of the TLR7 Binding | | |
| Site #2 motif from ORN7023. | | |
| ORN7024 | C*G*G*C*U*C* | 25 |
| (7024_TLR7_GH_R2153_pto) | G*G*C*A*G*A* | |
| ORN7024 is equivalent to | A*G*C*C*G*G* | |
| ORN7023, except that | G*C*C | |
| phosphodiester linkages are | | |
| replaced with the indicated | | |
| phosphorothioate linkages. | | |
| Underlined is the TLR7 | | |
| Binding Site #2 motif from | | |
| ORN7023, modified to include | | |
| the indicated | | |
| phosphorothioate | | |
| modifications. | | |

The asterisk (*) indicates a phosphorothioate (pto) modification between the indicated nucleosides; “m” indicates a 2′O-methyl modification; “f”′ indicates a 2′-fluoro modification; “-” indicates phosphodiester linkages between the indicated nucleosides.

ORN7005 Modifications

ORN7005, derived from pU1C (see, Zhang et al., 2018, Cell Reports 25, 3371-3381), has the nucleotide sequence:

(SEQ ID NO: 3)
U-C-U-C-U-C-U-C-U-C-U-C-U-C-U-C-U-C-U-C

In the nucleotide sequence of ORN7005 (SEQ ID NO: 3), all C residues were modified with either 2′O-methyl modifications or 2′-fluoro modifications to avoid cleavage and preserve unmodified U residues. This resulted in the 2′O-methyl-modified ORN7006 (SEQ ID NO: 26), and the 2′-fluoro-modified ORN7007 (SEQ ID NO: 27). In addition, to test whether the limited RNase resistance conferred by phosphorothioate modification could increase activity, a fully phosphorothioate-modified version of ORN7005 was generated, resulting in ORN7008 (SEQ ID NO: 28).

The modified ORNs derived from ORN7005 are summarized in Table 5.

TABLE 5
Modifications of ORN7005 to generate
TLR7-selective ligands.

| | | SEQ |
|---|---|---|
| Sequence | | ID |
| Description | Sequence | NO |
| ORN7006 | U-mC-U-mC-U- | 26 |
| (7006_TLR7_ | mC-U-mC-U-mC- | |
| ZZ_pU1C_ome) | U-mC-U-mC-U- | |
| | mC-U-mC-U-C | |
| ORN7007 | U-fC-U-fC-U- | 27 |
| (7007_TLR7_ | fC-U-fC-U-fC- | |
| ZZ_pU1C_F) | U-fC-U-fC-U- | |
| | fC-U-fC-U-C | |
| ORN7008 | U*C*U*C*U*C* | 28 |
| (7008_TLR7_ | U*C*U*C*U*C* | |
| ZZ_pU1C_pto) | U*C*U*C*U*C | |
| | *U*C | |

The asterisk (*) indicates a phosphorothioate (pto) modification between the indicated nucleosides; “m” indicates a 2′O-methyl modification; “f” indicates a 2′-fluoro modification; “-” indicates phosphodiester linkages between the indicated nucleosides.

ORN7009 Modifications

ORN7009, derived from pU2C (see, Zhang et al., 2018, Cell Reports 25, 3371-3381), has the nucleotide sequence:

(SEQ ID NO: 4)
C-C-U-U-C-U-U-C-U-U-C-U-U-C-U-U-C-U-U-C

The nucleotide sequence of ORN7009 (SEQ ID NO: 4) contains multiple U-U-C repeats. The first U in each U-U-C repeat in ORN7009 was modified with either 2′O-methyl or 2′-fluoro modifications. This resulted in the 2′O-methyl-modified ORN71 (SEQ ID NO: 29), with the modified mU-U-C repeat; and the 2′-fluoro-modified ORN7011 (SEQ ID NO: 30), with the modified f-U-C repeat. In addition, to test whether the limited RNase resistance conferred by phosphorothioate modification could increase activity, a fully phosphorothioate-modified version of ORN7009 was generated, resulting in ORN7012 (SEQ ID NO: 31).

The modified ORNs derived from ORN7009 are summarized in Table 6.

TABLE 6
Modifications of ORN7009 to generate
TLR7-selective ligands.

| | | SEQ |
|---|---|---|
| Sequence | | ID |
| Description | Sequence | NO |
| ORN7010 | C-C-mU-U-C-mU-U- | 29 |
| (7010_TLR7_ZZ_pU2C_ome) | C-mU-U-C-mU-U-C- | |
| ORN7010 includes several | mU-U-C-mU-U-C | |
| repeats of the | | |
| mU-U-C motif. | | |
| ORN7011 | C-C-fU-U-C-fU-U- | 30 |
| (7011_TLR7_ZZ_pU2C_F) | C-fU-U-C-fU-U-C- | |
| ORN7011 includes several | fU-U-C-fU-U-C | |
| repeats of the | | |
| fU-U-C motif. | | |
| ORN7012 | C*C*U*U*C*U*U*C* | 31 |
| (7012_TLR7_ZZ_pU2C_pto) | U*U*C*U*U*C*U | |
| ORN7012 includes several | *U*C*U*U*C | |
| repeats of the U-U-C | | |
| motif, but the | | |
| phosphodiester | | |
| linkages in | | |
| the motif have | | |
| been replaced | | |
| with | | |
| phosphorothioate linkages. | | |

The asterisk (*) indicates a phosphorothioate (pto) modification between the indicated nucleosides; “m” indicates a 2′O-methyl modification; “f” indicates a 2′-fluoro modification; “-” indicates phosphodiester linkages between the indicated nucleosides.

ORN7001 Modifications

The nucleotide sequence of ORN7001 (SEQ ID NO: 5), which was designed de novo as described above, contains two C-U-U-C motifs:

(SEQ ID NO: 5)
C-A-G-A-C-C-U-U-C-C-A-G-A-C-C-U-U-C

The first U in the C-U-U-C motifs in ORN7001 was modified with 2-fluoro or 2′-O-methyl modifications, resulting in ORN7002 (SEQ ID NO: 33), with the modified motif C-mU-U-C; and ORN7003 (SEQ ID NO: 34) with the modified motif C-fU-U-C. In addition, a modified ORN was generated from ORN7001 in which the entire C-U-U-C motif was modified with phosphorothioates, resulting in ORN7004 (SEQ ID NO: 35), with the modified motif C*U*U*C.

The modified ORNs derived from ORN7001 are summarized in Table 7.

TABLE 7
Modifications of ORN7001 to generate
TLR7-selective ligands.

| | | SEQ |
|---|---|---|
| Sequence | | ID |
| Description | Sequence | NO |
| ORN7002 | C-A-G-A-C-C-mU-U- | 33 |
| (7002_TLR7_AH_ome) | C-C-A-G-A-C-C-mU- | |
| The underlined C-mU-U-C | U-C | |
| motifs correspond to SEQ ID | | |
| NO: 36, and are 2′O-methyl- | | |
| modified versions | | |
| of the C-U- | | |
| U-C motifs in ORN7001. | | |
| ORN7003 | C-A-G-A-C-C-fU-U- | 34 |
| (7003_TLR7_AH_F) | C-C-A-G-A-C-C-fU- | |
| The underlined C-fU-U-C | U-C | |
| motifs correspond to SEQ ID | | |
| NO: 37, and are 2′-fluoro- | | |
| modified versions | | |
| of the C-U- | | |
| U-C motifs in ORN7001. | | |
| ORN7004 | C-A-G-A-C-C*U*U* | 35 |
| (7004_TLR7_AH_pto) | C-C-A-G-A-C- | |
| The underlined C*U*U*C | C*U*U*C | |
| motifs correspond to SEQ ID | | |
| NO: 38, and are | | |
| phosphorothioate-modified | | |
| versions of the | | |
| C-U-U-C motifs | | |
| in ORN7001. | | |

The asterisk (*) indicates a phosphorothioate (pto) modification between the indicated nucleosides; “m” indicates a 2′O-methyl modification; “f” indicates a 2′-fluoro modification; “-” indicates phosphodiester linkages between the indicated nucleosides.

### Example 2: Generation and Functional Characterization of TLR7-Selective RNA Ligands

This Example describes experiments that evaluated the TLR7-selectivity and activity of RNA ligands described in Example 1. The results of these experiments showed that certain modified RNA ligands derived from ORN7013 (derived from Forsbach et al., J Immunology (2008) 180:3729-3738) and ORN7023 (derived from WO2010/105819) had enhanced TLR7-selectivity and activity.

### Materials and Methods

RNA Production

RNA oligomers were synthesized and HPLC-purified by Integrated DNA Technologies (IDT).

For the production of IVT4, DNA oligonucleotides IVT4-fwd (5′ T-T-G-T-A-A-T-A-C-G-A-C-T-C-A-C-T-A-T-A-G-G-G-A-C-G-C-T-G-A-C-C-C-A-G-A-A-G-A-T-C-T-A-C-T-A-G-A-A-A-T-A-G-T-A-G-A-T-C-T-T-C-T-G-G-G-T-C-A-G- C-G-T-C-C-C 3′; SEQ ID NO: 65) and IVT4-rev (5′ G-G-G-A-C-G-C-T-G-A-C-C-C-A-G-A-A-G-A-T-C-T-A-C-T-A-T-T-T-C-T-A-G-T-A-G-A-T-C-T-T-C-T-G-G-G-T-C-A-G-C-G-T-C-C-C-T-A-T-A-G-T-G-A-G-T-C-G-T-A-T-T-A-C-A-A 3′; SEQ ID NO: 66) were annealed in annealing buffer (25 mM Tris-HCl, 25 mM NaCl, pH 7.4) by heating for 3 minutes to 95° C. and cooling to room temperature over a course of 15 minutes. This template was then employed in in vitro transcription using the TranscriptAid T7 High Yield Transcription Kit following the manual. RNA was then purified using mini Quick Spin DNA Columns (Roche).

PBMC Isolation

Peripheral blood mononuclear cells (PBMCs) were obtained from buffy coats from healthy adult donors. Buffy coat samples were first diluted with an equal volume of phosphate-buffered saline (PBS). Next, PBMCs were isolated via density gradient centrifugation with Lymphoprep density gradient media (STEMCELL Technologies) and SepMate-50 tubes (STEMCELL Technologies). Samples were centrifuged at 1000 g for 15 minutes. After centrifugation, the upper layers were collected by pouring into a new tube, diluted with PBS, and centrifuged at 450×g for 5 minutes. To lyse red blood cells, cell pellets were resuspended with 10 mL Ammonium-Chloride-Potassium (ACK) Lysis Buffer and incubated for 8 minutes at room temperature. After lysis, cells were washed with PBS and passed through two 70 μm cell strainers. Viable cell counts were determined with a Vi-CELL cell counter (Beckman Coulter). PBMCs were plated into 96-well round-bottom plates at a concentration of 4×10⁵ cells per well in RPMI media containing 10% heat-inactivated fetal bovine serum, 1% L-glutamine, and 1× Pen/Strep.

pDC Depletion

For plasmacytoid dendritic cell (pDC) depletion experiments, PBMCs were isolated as described above. pDCs were depleted using the CD303 Microbead Kit, human (Miltenyi Biotec).

PBMC Stimulation

Guanosine was added between about 20 and about 30 minutes before the addition of poly-L-arginine (p-L-arginine)-complexed RNA.

To generate p-L-arginine-RNA complexes, 0.2 μg RNA were added to 15 μl PBS, and 0.38 μg p-L-arginine (>70000 kDa, Sigma #P3892) were added (per well). After mixing by pipetting up and down, the reaction was incubated for 10 minutes, pipetted up and down again, and then added to the cells. In some experiments, RNA ligands were used at a final concentration of 0.6 μg/ml. Where indicated as “+2′3′ cGMP”, 2′3′ cyclic GMP purchased from eMolecules was complexed together with the RNA ligands. The final concentration of 2′3′ cyclic GMP was 5 μg/ml. p-L-arginine was adjusted accordingly to keep the mass ratio of nucleic acid:p-L-arginine constant.

For Lipofectamine 2000 transfection of IVT4, per reaction, 0.5 μl Lipofectamine 2000 (Thermo Fisher Scientific) were added to 25 μl Opti-MEM. The mixture was mixed well and incubated 5 minutes at room temperature. During the incubation, 2 ng of IVT4 were added to 25 μl Opti-MEM. The Lipofectamine/Opti-MEM mix was added to the diluted IVT4 and mixed by pipetting up and down. The mixture was incubated 20 minutes at room temperature and then added to the cells.

To generate DOTAP-RNA complexes, master mixes containing DOTAP and Opti-MEM or RNA and Opti-MEM were made and incubated for 5 minutes at room temperature. The two master mixes were then combined at a final RNA:DOTAP mass ratio of 1:5. The mixtures were then incubated for 20 minutes at room temperature before adding to the cells.

Cytokine Detection

Cell culture supernatants were collected 15-18 hours after stimulation and stored at −80° C. Cytokines in cell culture supernatants were detected with the BD OptEIA human TNF ELISA Set (BD Biosciences), and either Invitrogen IFN alpha Human Matched Antibody Pair kit (Thermo Fisher Scientific) or Human IFN Alpha Multi-Subtype ELISA Kit (PBL Assay Science).

Particle Distribution

Particle sizes were determined by Dynamic Light Scattering (Wyatt Technology). Samples were placed in multiple wells in a 384-well plate and each well was measured ten times for 5 seconds. Z-averages were reported.

RNase Cleavage

Cleavage assays were performed as described in Ostendorf et al., (2020) 52(4):591-605, with the following modifications. 0.5 μg of each RNA ligand were incubated with recombinant RNase T2 (Origene) in a 15 μl reaction. A four-point time course was performed where reactions were incubated for 3, 6, 9, and 12 minutes. Reactions were stopped with 100 mM Tris-HCL (pH 7.5). Upon addition of 2×TBE-urea sample buffer (Thermo LC6876), the reactions were incubated for 3 minutes at 70° C. The reactions were then loaded into 15% TBE-Urea PAGE gels and run for 1.5 hours at 150V. Gels were stained with SYBR Gold (Invitrogen) at 1:10K.

### Results

Certain Modified RNA Ligands have Enhanced TLR7-Selectivity and Activity in PBMCs

To test the effects of the RNA ligands described in Example 1 on TLR7 and TLR8 activation, PBMCs were stimulated with the RNA ligands using p-L-arginine complex delivery, delivery with DOTAP, or delivery without a carrier. Activity of TLR7 and TLR8 was assessed based on measurements of IFN-α or TNF-α by enzyme-linked immunosorbent assays (ELISA).

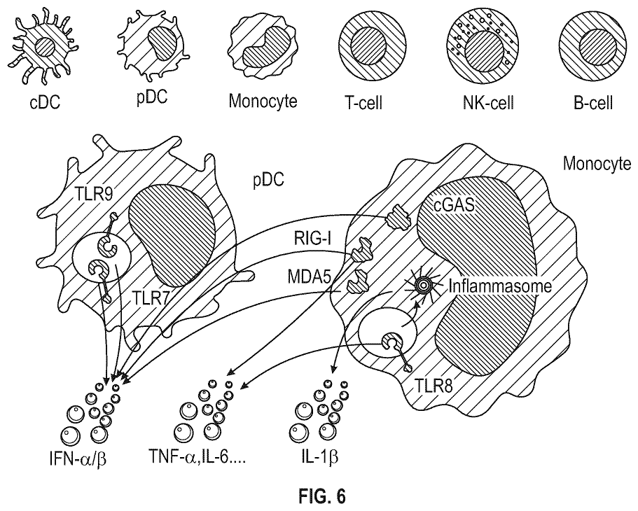

In this setting, as depicted in FIG. 6, IFN-α induced by small RNAs is derived from TLR7 expressed by pDCs, or from RIG-I expressed by monocytes. In contrast, TNF-α secretion is derived from monocytes and serves as an indication of TLR8 activation. As shown in this figure, the p-L-arginine delivery restricts most of the activation to TLRs.

p-L-Arginine Delivery

First, the effects of the RNA ligands on TLR7 and TLR8 activation using p-L-arginine complex delivery were tested.

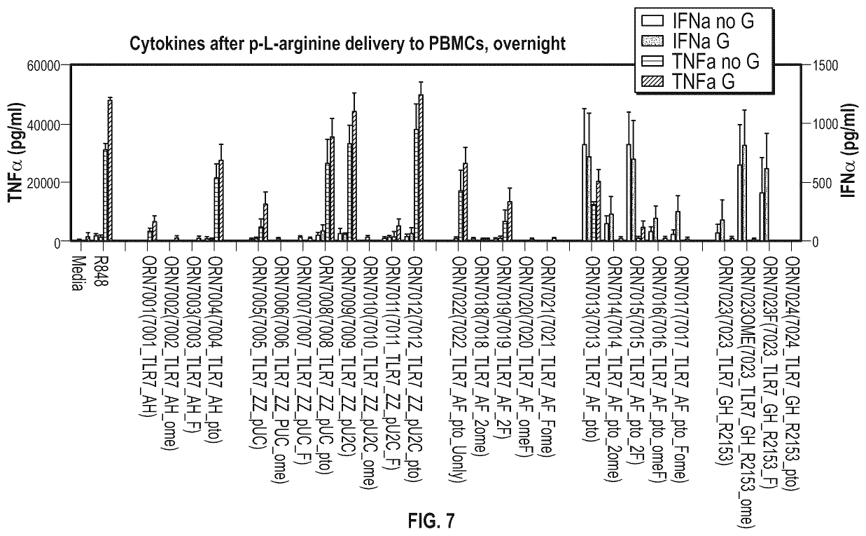

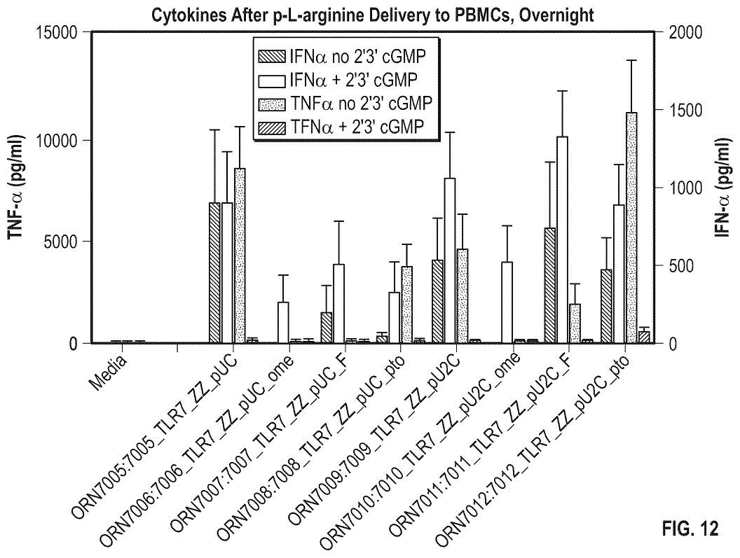

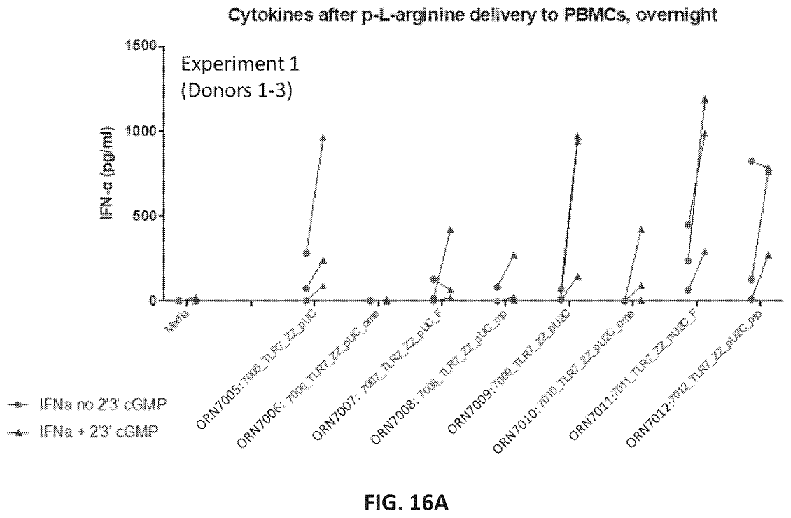

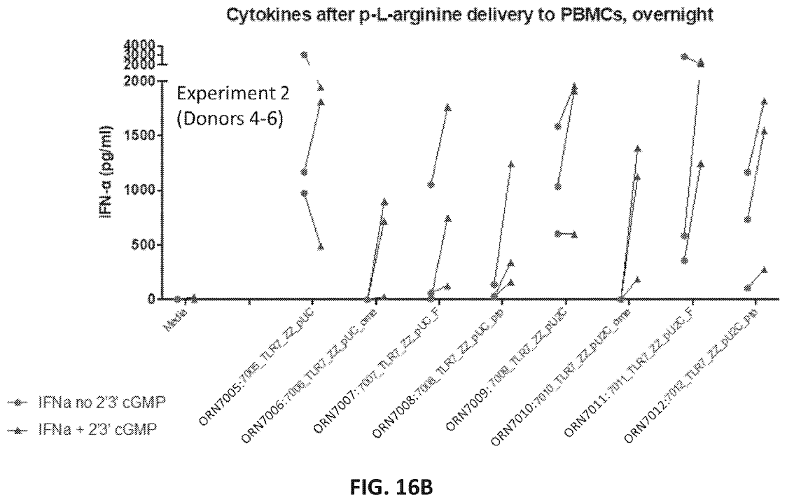

As shown in FIG. 7, ORN7005 (derived from pU1C of Zhang et al., 2018), ORN7009 (derived from pU2C of Zhang et al., 2018), and ORN7001 (de novo designed ligand) induced no or only minor IFN-α secretion, irrespective of modifications, indicating limited TLR7 activity. In addition, several modified ORNs derived from ORN7005, ORN7009, and ORN7001 showed marked TLR8 activity, as evidenced by TNF-α secretion. ORN7005-7008 and ORN7009-7012 were predicted to show TLR7 activity when exogenous guanosine was provided as a ligand for TLR7 Binding Site #1. The observed lack of IFN-α secretion in this condition was unexpected. To rule out technical error, the experiment for ORN7005-7008 and ORN7009-7012 was repeated with the following modifications: (1) guanosine was replaced with the guanosine derivative 2′3′ cyclic GMP (2′3′ cGMP), which mimics an RNA degradation product; (2) the 2′3′ cGMP was included in the p-L-arginine complexes rather than adding directly to cell culture media; and (3) a more sensitive ELISA kit was used for detection of IFN-α. As shown in FIG. 12, the capacity to activate TLR7 and induce IFN-α secretion was markedly enhanced for ORN7005-7008 and ORN7009-7012 when co-delivered with 2′3′ cGMP as a ligand for TLR7 Binding Site #1. Of note, there was variability across donors and experiments; specifically, one donor showed an unexpectedly high IFN-α response to ORN7005 alone (FIGS. 16A-16B). However, taking the data as a whole, the trend towards increased IFN-α production when RNA ligands are paired with a guanosine analog remains consistent. The 2′-fluoro- and 2′O-methyl-modified versions (i.e., ORN7006, ORN7007, ORN7010, ORN7011) demonstrated robust IFN-a induction with little to no measurable TNF-α when co-delivered with 2′3′ cyclic GMP, indicating TLR7-selectivity.

In contrast, as shown in FIG. 7, the limited TLR7 selectivity and activity of ORN7013 (derived from Forsbach et al., J Immunology (2008) 180:3729-3738) were confirmed. Furthermore, the modifications introduced in ORN7014-7017 (modified versions of ORN7013) were found to further increase TLR7 selectivity compared to ORN7013, as shown by the maintained (or lower) IFN-α secretion levels and greatly reduced TNF-α secretion levels relative to ORN7013. The phosphodiester-linked ORNs derived from ORN7013 (ORN7022 and ORN7018-7021) lacked TLR7 activity when delivered using poly-L-arginine.

Similarly, as shown in FIG. 7, the introduction of modifications into ORN7023 (derived from WO2010/105819) maintained TLR7 selectivity while further increasing the activity (see ORN7023F and ORN70230me in FIG. 7). However, ORN7024, the phosphorothioate-modified RNA ligand derived from ORN7023 had reduced activity.

DOTAP Delivery

Next, the effects of the RNA ligands on TLR7 and TLR8 activation using DOTAP delivery were tested at two different concentrations of RNA ligands, 0.6 μg/ml and 2 μg/ml.

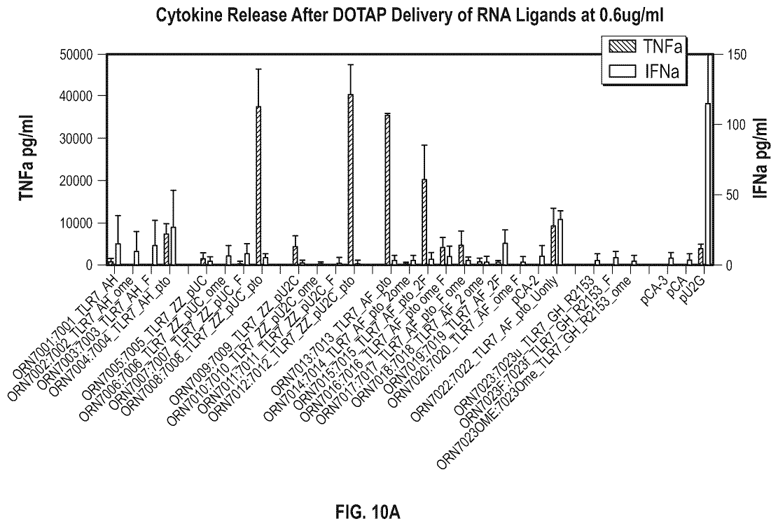

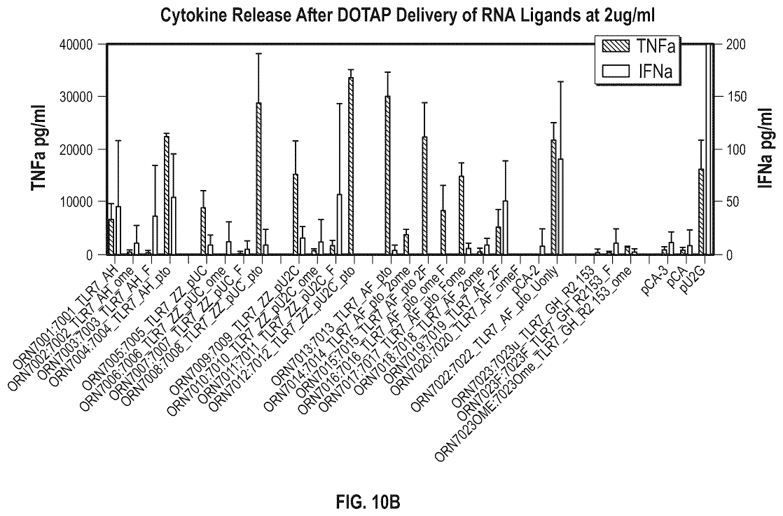

As shown in FIGS. 10A-10B, ORN7001 (de novo designed ligand) induced TLR7 and TLR8 activity, indicated by IFN-α and TNF-α secretion, respectively. ORN7002, which is a 2′O-methyl modified version of ORN7001, had reduced activity. In contrast, ORN7003, which is a 2′-fluoro-modified version of ORN7001, maintained TLR7-activity relative to ORN7001, while eliminating TLR8 activity. ORN7004, which is a phosphorothioate-linked version of ORN7001, had mildly increased TLR7 activity and markedly increased TLR8 activity.

ORN7005 (derived from pU1C of Zhang et al., 2018) and ORN7009 (derived from pU2C of Zhang et al., 2018) activated TLR8 rather than TLR7. Phosphorothioate modifications of ORN7005 and ORN7009 (i.e., ORN7008 and ORN7012, respectively), further increased TLR8 activation. The 2′O-methyl-modified versions of ORN7005 and ORN7009 (i.e., ORN7006 and ORN7010, respectively) reduced both TLR8 and TLR7 activation. In addition, the 2′fluoro-modified version of ORN7005 (i.e., ORN7007) also reduced activity. ORN7011, which is a 2′-fluoro-modified version of ORN7009, had TLR7-activity at the higher concentration of 2 μg/ml (FIG. 10B), without TLR8 activity. Importantly, the TLR7 activity of these RNA ligands (i.e., ORN7005-ORN7012) may be suboptimal in this setting due to the absence of guanosine or a guanosine derivative to act as a ligand for TLR7 Binding Site #1. Co-delivery of guanosine or a guanosine derivative with RNA ligand-DOTAP complexes may thus enhance TLR7 activity, as was observed in the setting of p-L-arginine complex delivery (FIG. 12).

ORN7013 (derived from Forsbach et al., J Immunology (2008) 180:3729-3738) and its modified versions ORN7014-ORN7017 either activated TLR8 and not TLR7, or had no activity. ORN7019, which is a 2′-fluoro-modified and phosphodiester-linked version of ORN7013, showed selective TLR7 activation at the concentration of 0.6 μg/ml (FIG. 10A). However, ORN7019 also had TLR8-activity at the concentration of 2 μg/ml (FIG. 10B). ORN7022, which is a phosphodiester-linked version of ORN7013, activated both TLR7 and TLR8 at both the 0.6 μg/ml and 2 μg/ml concentrations (FIGS. 10A-10B). All other RNA ligands that were based on ORN7013 (derived from Forsbach et al., J Immunology (2008) 180:3729-3738) led to low or no TLR7 activation when delivered using DOTAP.

Thus, these results show that when delivered with DOTAP, a different spectrum of RNA ligands had the highest activity as compared to delivery with poly-L-arginine.

Carrier-Free Delivery

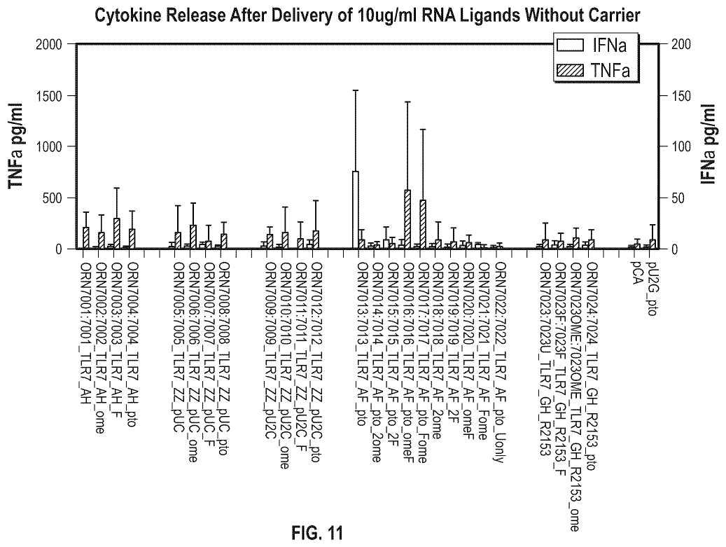

Finally, the effects of the RNA ligands on TLR7 and TLR8 activation using carrier-free delivery were tested at a concentration of RNA ligands of 10 μg/ml. As shown in FIG. 11, none of the RNA ligands showed reproducible TLR7 or TLR8 activation when delivered without a carrier.

The Activity of Modified RNA Ligands is Specific to TLR7 Expressed in pDCs

In PBMCs, IFN-α secretion upon TLR7 activation is exclusively derived from pDCs, while IFN-α secretion upon activation of RIG-I (a sensor of short double-stranded 5′triphosphate RNA) is dependent on monocytes. Therefore, to confirm the TLR7-versus RIG-I-specificity of the RNA ligands described above, pDCs were depleted from PBMCs before transfection of the RNA ligands.

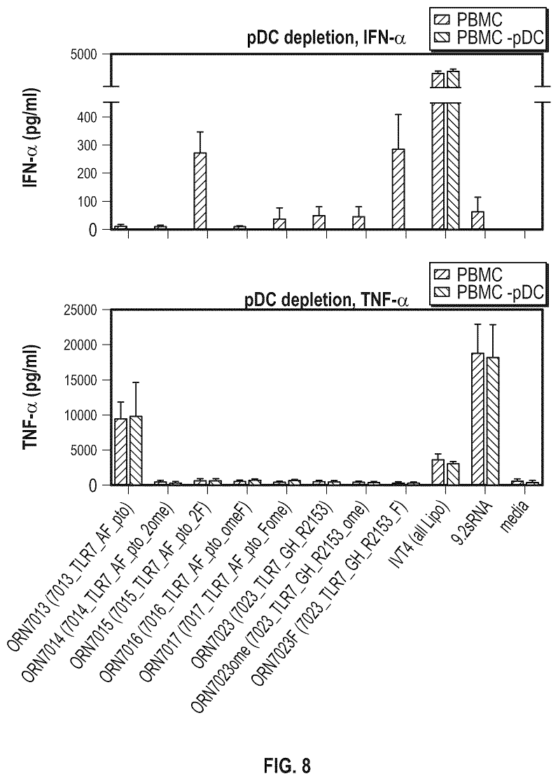

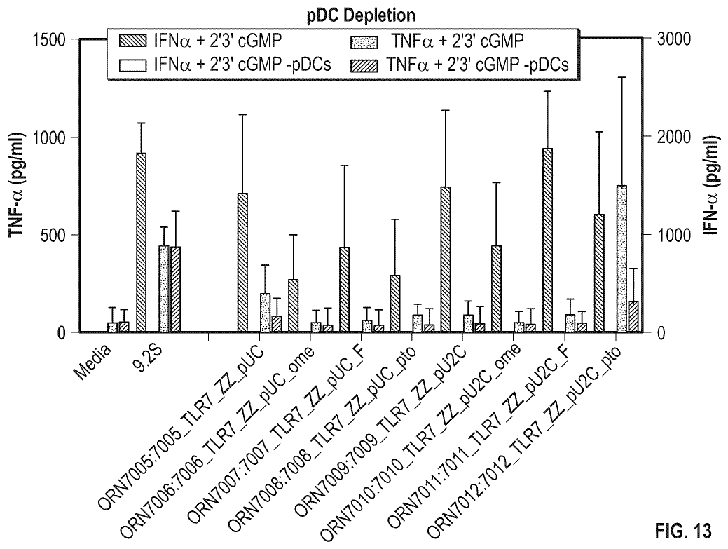

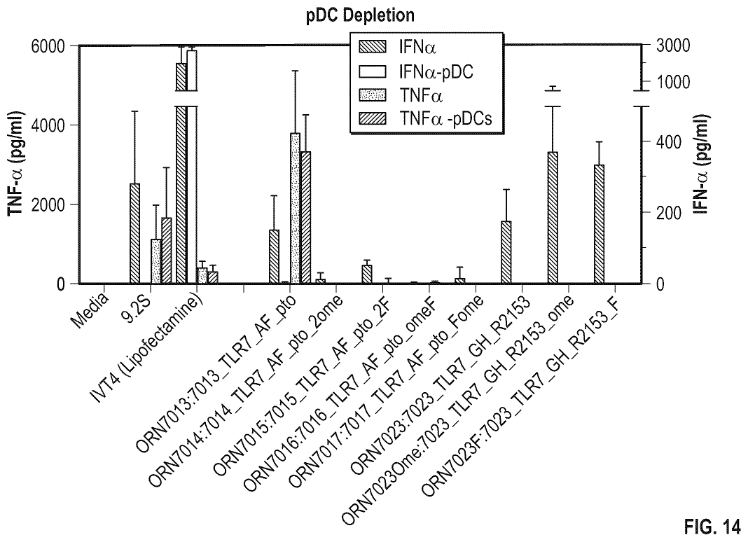

As shown in FIGS. 8 and 13-14, secretion of IFN-α was abrogated by pDC depletion, indicating that activation of TLR7 by the RNA ligands, and not of RIG-I, was responsible for the secretion of IFN-α. Since RIG-I is mainly expressed by monocytes, IFN-α secretion after lipofectamine-mediated transfection of the RIG-I ligand 3P-RNA (IVT4) was maintained after pDC depletion.

The Activity of Modified RNA Ligands is not Caused by Structural Differences of Nanoparticles

The generation of IFN-α versus IL-12p70 following stimulation by oligonucleotides complexed with poly-L-arginine has been previously reported to be time-dependent, possibly due to a change in the size of particles, see, e.g., Wimmenauer V. Identifikation immunstimulatorischer Nukleinsäureliganden zur Stimulation von Rezeptoren der angeborenen Immunantwort. 2009. Dissertation, Rheinische Friedrich-Wilhelms-Universität Bonn. Online edition available at the website: nbn-resolving.org/urn:nbn:de:hbz:5N-17312 (see, in particular pages 62 and 66).

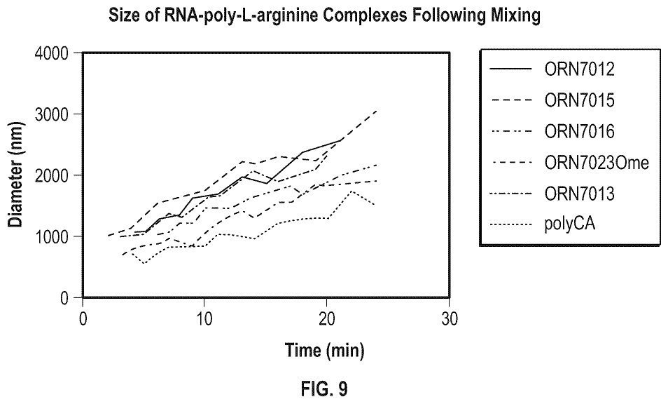

To determine whether the generation of IFN-α or TNF-α induced by specific sequences was due to differences in particle size, the particle sizes of various RNA-poly-L-arginine complexes were analyzed using Dynamic Light Scattering. As shown in FIG. 9, particles containing the TLR7-selective and highly active ORN70230me behaved similarly to particles containing the TLR8-active ORN7012, the less TLR7-selective and active ORN7013, the moderately selective and active ORN7015, the TLR7-selective but weakly active ORN7016, and the negative control pCA. Because the incubation time was the same for all oligoribonucleotides (10 minutes) and because their diameters were similar at the 10 minute time point, the results shown in FIG. 9 suggest that the range of TLR7 selectivity and activity seen among the different oligoribonucleotides is not due to a difference in particle size.

RNase-Mediated Degradation of Modified RNA Ligands is Inhibited by 2′Fluoro- and 2′-O-Methyl Modifications

To assess whether RNA modifications at the 2′ position interfere with RNAse cleavage activity, a series of in vitro cleavage assays were performed by incubating recombinant RNase T2 with various RNA ligands. RNAse T2 is expressed ubiquitously, and importantly, it is active in the cell types that express TLR7. In addition, RNAse T2 has been identified as important for the activation of TLR8 (Ostendorf et al., (2020) 52(4):591-605).

A time course was performed where each RNA ligand was incubated with an optimized amount of RNase T2, and the degradation products from each reaction were visualized on a TBE-Urea PAGE gel. ORNs 7013-7017 and 7009-7012 were tested.

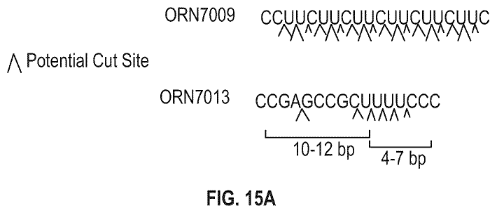

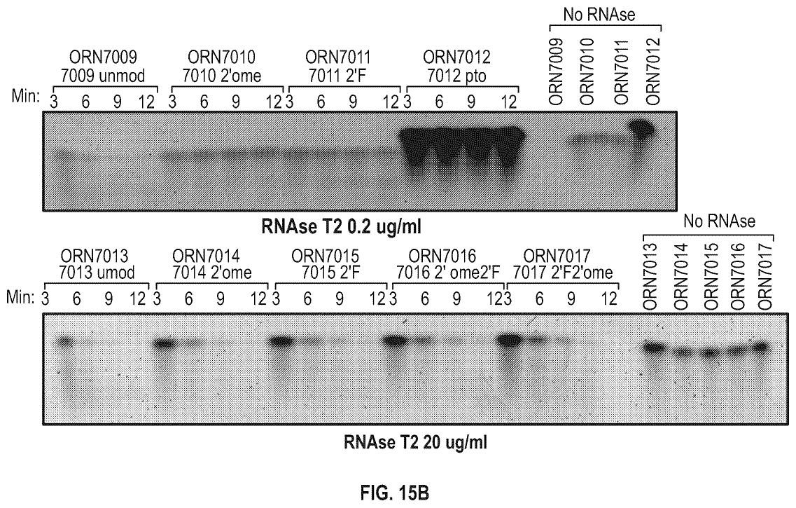

In each case, the unmodified RNA ligands (i.e., ORN7013 and ORN7009) were degraded more quickly, as seen by a decrease in full RNA ligand levels on the gel (FIGS. 15A-15B). Notably, ORNs 7010-7012 showed stronger protection against RNase activity, likely due to the fact that they have a greater proportion of 2′-modified uridines due to their sequence as compared to ORNs 7014-7017 (FIG. 15B).

### Sequences

TABLE 8

| | | SEQ |
|---|---|---|
| Sequence | | ID |
| Description | Sequence | NO |
| ORN7013 | C*C*G*A*G*C*C*G*C*U*U*U*U | 1 |
| (7013_TLR7_AF_pto) | *C*C*C | |
| ORN7023 | C-G-G-C-U-C-G-G-C-A-G-A-A-G-C- | 2 |
| (7023u_TLR7_GH_R2153) | C-G-G-G-C-C | |
| ORN7005 | U-C-U-C-U-C-U-C-U-C-U-C-U-C-U- | 3 |
| (7005_TLR7_ZZ_pUC) | C-U-C-U-C | |
| ORN7009 | C-C-U-U-C-U-U-C-U-U-C-U-U-C-U- | 4 |
| (7009_TLR7_ZZ_pU2C) | U-C-U-U-C | |
| ORN7001 | C-A-G-A-C-C-U-U-C-C-A-G-A-C-C- | 5 |
| (7001_TLR7_AH) | U-U-C | |
| RNA trimer degradation product; N | N-U-N | N/A |
| represents any ribonucleotide. | | |
| Unmodified TLR7/TLR8 motif in | C*U*U*U*U*C | N/A |
| ORN7013 | | |
| Degradation product of TLR7/TLR8 | C*U*U | N/A |
| motif in ORN7013 | | |
| Degradation product of TLR7/TLR8 | U*U*U | N/A |
| motif in ORN7013 | | |
| Degradation product of TLR7/TLR8 | U*U*C | N/A |
| motif in ORN7013 | | |
| Modified TLR7/TLR8 motif in | C*X-U*X-U*C | N/A |
| ORN7013 | | |
| (X represents either 2′-Fluoro-U or | | |
| 2′O-methyl-U) | | |
| ORN7014 | C*C*G*A*G*C*C*G*C*mU- | 12 |
| (7014_TLR7_AF_pto_2ome) | U*mU-U*C*C*C | |
| ORN7015 | C*C*G*A*G*C*C*G*C*fU-U*fU- | 13 |
| (7015_TLR7_AF_pto_2F) | U*C*C*C | |
| ORN7016 | C*C*G*A*G*C*C*G*C*mU-U*fU- | 14 |
| (7016_TLR7_AF_pto_omeF) | U*C*C*C | |
| ORN7017 | C*C*G*A*G*C*C*G*C*fU-U*mU- | 15 |
| (7017_TLR7_AF_pto_Fome) | U*C*C*C | |
| ORN7022 | C-C-G-A-G-C-C-G-C*U*U*U*U*C- | 16 |
| (7022_TLR7_AF_pto_Uonly) | C-C | |
| ORN7018 | C-C-G-A-G-C-C-G-C-mU-U*mU- | 17 |
| (7018_TLR7_AF_2ome) | U*C-C-C | |
| ORN7019 | C-C-G-A-G-C-C-G-C-fU-U*fU-U*C- | 18 |
| (7019_TLR7_AF_2F) | C-C | |
| ORN7020 | C-C-G-A-G-C-C-G-C-mU-U*fU- | 19 |
| (7020_TLR7_AF_omeF) | U*C-C-C | |
| ORN7021 | C-C-G-A-G-C-C-G-C-fU-U*mU- | 20 |
| (7021_TLR7_AF_Fome) | U*C-C-C | |
| ORN7023 TLR7 Binding Site #2 | C-U-C | N/A |
| motif. | | |
| ORN7023 TLR7 Binding Site #2 | D-U-C | N/A |
| modified motif. | | |
| “D” represents either 2′-Fluoro-C or | | |
| 2′O-methyl-C. | | |
| ORN7023F | C-G-G-fC-U-C-G-G-C-A-G-A-A-G- | 23 |
| (7023F_TLR7_GH_R2153_F) | C-C-G-G-G-C-C | |
| ORN7023Ome | C-G-G-mC-U-C-G-G-C-A-G-A-A-G- | 24 |
| (7023Ome_TLR7_GH_R2153_ome) | C-C-G-G-G-C-C | |
| ORN7024 | C*G*G*C*U*C*G*G*C*A*G*A*A | 25 |
| (7024_TLR7_GH_R2153_pto) | *G*C*C*G*G*G*C*C | |
| ORN7006 | U-mC-U-mC-U-mC-U-mC-U-mC-U- | 26 |
| (7006_TLR7_ZZ_pUC_ome) | mC-U-mC-U-mC-U-mC-U-C | |
| ORN7007 | U-fC-U-fC-U-fC-U-fC-U-fC-U-fC-U- | 27 |
| (7007_TLR7_ZZ_pUC_F) | fC-U-fC-U-fC-U-C | |
| ORN7008 | U*C*U*C*U*C*U*C*U*C*U*C*U* | 28 |
| (7008_TLR7_ZZ_pUC_pto) | C*U*C*U*C*U*C | |
| ORN7010 | C-C-mU-U-C-mU-U-C-mU-U-C-mU- | 29 |
| (7010_TLR7_ZZ_pU2C_ome) | U-C-mU-U-C-mU-U-C | |
| ORN7011 | C-C-fU-U-C-fU-U-C-fU-U-C-fU-U- | 30 |
| (7011_TLR7_ZZ_pU2C_F) | C-fU-U-C-fU-U-C | |
| ORN7012 | C*C*U*U*C*U*U*C*U*U*C*U*U | 31 |
| (7012_TLR7_ZZ_pU2C_pto) | *C*U*U*C*U*U*C | |
| C/U-rich CUUC motif within | C-U-U-C | N/A |
| ORN7001, containing two potential | | |
| motifs for TLR7 Binding Site #2: | | |
| CUU and UUC. | | |
| ORN7002 | C-A-G-A-C-C-mU-U-C-C-A-G-A-C- | 33 |
| (7002_TLR7_AH_ome) | C-mU-U-C | |
| ORN7003 | C-A-G-A-C-C-fU-U-C-C-A-G-A-C- | 34 |
| (7003_TLR7_AH_F) | C-fU-U-C | |
| ORN7004 | C-A-G-A-C-C*U*U*C-C-A-G-A-C- | 35 |
| (7004_TLR7_AH_pto) | C*U*U*C | |
| 2′O-methyl-modified C/U-rich | C-mU-U-C | N/A |
| CUUC motif from ORN7002. | | |
| 2′-fluoro-modified C/U-rich CUUC | C-fU-U-C | N/A |
| motif from ORN7003. | | |
| Phosphorothioate -modified C/U-rich | C*U*U*C | N/A |
| CUUC motif from ORN7004. | | |
| UUC repeat sequence in ORN 7009 | U-U-C | N/A |
| 2′O-methyl-modified repeat | mU-U-C | N/A |
| sequence from ORN7010 | | |
| 2′-fluoro-modified repeat sequence | fU-U-C | N/A |
| from ORN7011 | | |
| 2′-fluoro-modified CUC motif from | fC-U-C | N/A |
| ORN7023F | | |
| 2′O-methyl-modified CUC motif | mC-U-C | N/A |
| from ORN7023Ome | | |
| 2′O-methyl-modified motif in | C*mU-U*mU-U*C | N/A |
| ORN7014 (derived from ORN7013). | | |
| 2′-fluoro-modified motif in | C*fU-U*fU-U*C | N/A |
| ORN7015 (derived from ORN7013) | | |
| 2′O-methyl- and 2′-fluoro-modified | C*mU-U*fU-U*C | N/A |
| motif in ORN7016 (derived from | | |
| ORN7013). | | |
| 2′-fluoro- and 2′O-methyl-modified | C*fU-U*mU-U*C | N/A |
| motif in ORN7017 (derived from | | |
| ORN7013). | | |
| Phosphorothioate- and 2′O-methyl- | C-mU-U*mU-U*C | N/A |
| modified motif in ORN7018 (derived | | |
| from ORN7013) | | |
| Phosphorothioate- and 2′-fluoro- | C-fU-U*fU-U*C | N/A |
| modified motif in ORN7019 (derived | | |
| from ORN7013). | | |
| Phosphorothioate-, 2′O-methyl-, and | C-mU-U*fU-U*C | N/A |
| 2′-fluoro-modified motif in | | |
| ORN7020 (derived from ORN7013). | | |
| Phosphorothioate-, 2′-fluoro-, and | C-fU-U*mU-U*C | N/A |
| 2′O-methyl-modified motif in | | |
| ORN7021 (derived from ORN7013). | | |
| Binding Site #2 motif within the | X-U*C | N/A |
| modified C*XU*XU*C motif from | | |
| ORN7013. X represents either 2′- | | |
| Fluoro-U or 2′O-methyl-U. | | |
| Binding Site #2 motif within the | X-U*X | N/A |
| modified C*XU*XU*C motif from | | |
| ORN7013. X represents either 2′- | | |
| Fluoro-U or 2′O-methyl-U. | | |
| TLR7 Binding Site #2 motif within | C-U-U | N/A |
| the C/U-rich CUUC motif of | | |
| ORN7001. | | |
| TLR7 Binding Site #2 motif within | U-U-C | N/A |
| the C/U-rich CUUC motif of | | |
| ORN7001. | | |
| 9.2S RNA | A-G-C-U-U-A-A-C-C-U-G-U-C-C-U- | 56 |
| | U-C-A-A | |
| Motif for ORN7014-7017. | C₁*U₂-U₃*U₄-U₅*C₆ | N/A |
| U2 has a 2′O-methyl modification | | |
| (mU) or a 2′-fluoro modification | | |
| (fU), and U4 has a 2′O-methyl | | |
| modification (mU) or a 2′-fluoro | | |
| modification (fU) | | |
| 5′ Nucleotide sequence for | C*C*G*A*G*C*C*G | N/A |
| ORN7014-7017. | | |
| 3′ Nucleotide sequence for | C*C | N/A |
| ORN7014-7017. | | |
| 5′ Nucleotide sequence for | C-G-G | N/A |
| ORN7023F, ORN7023Ome | | |
| 3′ Nucleotide sequence for | G-G-C-A-G-A-A-G-C-C-G-G-G-C-C | 61 |
| ORN7023F, ORN7023Ome | | |
| IVT4 Oligo Nucleotide Sequence | G-G-G-A-C-G-C-U-G-A-C-C-C-A-G- | 62 |
| | A-A-G-A-U-C-A-C-U-A-G-A-A-A- | |
| | U-A-G-U-A-G-A-U-C-U-U-C-U-G- | |
| | G-G-U-C-A-G-C-G-U-C-C-C | |
| RNA trimer degradation product; N | N*U*N | N/A |
| represents any ribonucleotide. | | |
| RNA trimer degradation product; N | N-U*N | N/A |
| represents any ribonucleotide. | | |
| IVT4-fwd oligo | T-T-G-T-A-A-T-A-C-G-A-C-T-C-A- | 65 |
| | C-T-A-T-A-G-G-G-A-C-G-C-T-G-A- | |
| | C-C-C-A-G-A-A-G-A-T-C-T-A-C-T- | |
| | A-G-A-A-A-T-A-G-T-A-G-A-T-C-T- | |
| | T-C-T-G-G-G-T-C-A-G-C-G-T-C-C- | |
| | C | |
| IVT4-rev oligo | G-G-G-A-C-G-C-T-G-A-C-C-C-A-G- | 66 |
| | A-A-G-A-T-C-T-A-C-T-A-T-T-T-C- | |
| | T-A-G-T-A-G-A-T-C-T-T-C-T-G-G- | |
| | G-T-C-A-G-C-G-T-C-C-C-T-A-T-A- | |
| | G-T-G-A-G-T-C-G-T-A-T-T-A-C-A- | |
| | A | |
| First nucleotide sequence for | C-A-G-A-C | N/A |
| ORN7001-7004 | | |
| 5′ Nucleotide sequence for | C-C-G-A-G-C-C-G | N/A |
| ORN7018-7021. | | |
| GGG motif in the 3′ nucleotide | G-G-G | N/A |
| sequence for ORN7023F, | | |
| ORN7023Ome | | |
| pCA, pCA-2 and pCA-3 nucleotide | C-A-C-A-C-A-C-A-C-A-C-A-C-A-C- | 70 |
| sequence | A-C-A-C-A | |
| pU2G nucleotide sequence | G-G-U-U-G-U-U-G-U-U-G-U-U-G- | 71 |
| | U-U-G-U-U-G | |
| pU2G_pto nucleotide sequence | G*G*U*U*G*U*U*G*U*U*G*U*U | 72 |
| | *G*U*U*G*U*U*G | |

“*” indicates a phosphorothioate linkage between the indicated nucleosides; “-” indicates a phosphodiester linkage between the indicated nucleosides; “m” indicates a 2′O-methyl modification; “f” indicates a 2′-fluoro modification.

What is claimed is:

1. An immunomodulatory composition that selectively activates TLR7, comprising an RNA ligand and a pharmaceutically acceptable carrier, wherein the RNA ligand comprises:
(a) a TLR7-selective motif comprising Formula II:
C₁*U₂-U₃*U₄-U₅*C₆ (Formula II),
wherein C₁ and C₆ are cytidine nucleosides, and U₂, U₃, U₄, and U₅ are uridine nucleosides,
wherein U₂ has a 2′O-methyl modification (mU), a 2′-fluoro modification (fU), a 2′-amino modification, a 2′-deoxy modification, or a 2′-methoxyethoxy modification,
wherein U₄ has a 2′O-methyl modification (mU), a 2′-fluoro modification (fU), a 2′-amino modification, a 2′-deoxy modification, or a 2′-methoxyethoxy modification, and
wherein “*” represents a phosphorothioate linkage and “-” represents a phosphodiester linkage;
(b) a first nucleotide sequence linked to the 5′ end of the TLR7-selective motif; and
(c) a second nucleotide sequence linked to the 3′ end of the TLR7-selective motif.

2. The immunomodulatory composition of claim 1, wherein U₂ of the TLR7-selective motif has a 2′O-methyl modification (mU).

3. The immunomodulatory composition of claim 1, wherein U₄ of the TLR7-selective motif has a 2′O-methyl modification (mU).

4. The immunomodulatory composition of claim 1, wherein U₂ of the TLR7-selective motif has a 2′-fluoro modification (fU).

5. The immunomodulatory composition of claim 1, wherein U₄ of the TLR7-selective motif has a 2′-fluoro modification (fU).

6. The immunomodulatory composition of any one of claims 1-3, wherein U₂ and U₄ of the TLR7-selective motif have a 2′O-methyl modification (mU).

7. The immunomodulatory composition of any one of claims 1 and 4-5, wherein U₂ and U₄ of the TLR7-selective motif have a 2′-fluoro modification (fU).

8. The immunomodulatory composition of any one of claims 1-2 and 5, wherein U₂ of the TLR7-selective motif has a 2′O-methyl modification (mU), and U₄ of the TLR7-selective motif has a 2′-fluoro modification (RU).

9. The immunomodulatory composition of any one of claims 1 and 3-4, wherein U₂ of the TLR7-selective motif has a 2′-fluoro modification (fU), and U₄ of the TLR7-selective motif has a 2′O-methyl modification (mU).

10. The immunomodulatory composition of any one of claims 1-3, wherein the TLR7-selective motif comprises the nucleotide sequence of SEQ ID NO: 44.

11. The immunomodulatory composition of any one of claims 1 and 4-5, wherein the TLR7-selective motif comprises the nucleotide sequence of SEQ ID NO: 45.

12. The immunomodulatory composition of any one of claims 1-2 and 5, wherein the TLR7-selective motif comprises the nucleotide sequence of SEQ ID NO: 46.

13. The immunomodulatory composition of any one of claims 1 and 3-4, wherein the TLR7-selective motif comprises the nucleotide sequence of SEQ ID NO: 47.

14. An immunomodulatory composition that selectively activates TLR7, comprising an RNA ligand and a pharmaceutically acceptable carrier, wherein the RNA ligand comprises:
(a) a TLR7-selective motif comprising a nucleotide sequence selected from the group consisting of: C*mU-U*mU-U*C, C*fU-U*fU-U*C, C*mU-U*fU-U*C, and C*fU-U*mU-U*C, wherein “*” represents a phosphorothioate linkage, “-” represents a phosphodiester linkage, “m” represents a 2′O-methyl modification, and “f” represents a 2′-fluoro modification,
(b) a first nucleotide sequence linked to the 5′ end of the TLR7-selective motif, and
(c) a second nucleotide sequence linked to the 3′ end of the TLR7-selective motif.

15. The immunomodulatory composition of any one of claims 1-14, wherein the first nucleotide sequence comprises one nucleoside, or at least two nucleosides linked by phosphorothioate linkages,
wherein the first nucleotide sequence does not comprise a uridine nucleoside, and
wherein the first nucleotide sequence is linked to the TLR7-selective motif by a phosphorothioate linkage.

16. The immunomodulatory composition of claim 15, wherein the first nucleotide sequence comprises eight nucleosides linked by phosphorothioate linkages.

17. The immunomodulatory composition of any one of claims 1-16, wherein the first nucleotide sequence comprises the nucleotide sequence C*C*G*A*G*C*C*G, or a nucleotide sequence having at least about 85% identity to SEQ ID NO: 58,
wherein “*” represents a phosphorothioate linkage, and
wherein the first nucleotide sequence is linked to the TLR7-selective motif by a phosphorothioate linkage.

18. The immunomodulatory composition of any one of claims 1-17, wherein the second nucleotide sequence comprises one nucleoside, or at least two nucleosides linked by phosphorothioate linkages,
wherein the second nucleotide sequence does not comprise a uridine nucleoside, and
wherein the second nucleotide sequence is linked to the TLR7-selective motif by a phosphorothioate linkage.

19. The immunomodulatory composition of claim 18, wherein the second nucleotide sequence comprises two nucleosides linked by phosphorothioate linkages.

20. The immunomodulatory composition of any one of claims 1-19, wherein the second nucleotide sequence comprises the nucleotide sequence C*C,
wherein “*” represents a phosphorothioate linkage, and
wherein the second nucleotide sequence is linked to the TLR7-selective motif by a phosphorothioate linkage.

21. The immunomodulatory composition of any one of claims 1-3, 6, 10, and 14-20, wherein the RNA ligand comprises the nucleotide sequence of SEQ ID NO: 12, or a nucleotide sequence having at least about 85% identity to SEQ ID NO: 12 and comprising the TLR7-selective motif of SEQ ID NO: 44.

22. The immunomodulatory composition of any one of claims 1, 4-5, 7, 11, and 14-20, wherein the RNA ligand comprises the nucleotide sequence of SEQ ID NO: 13, or a nucleotide sequence having at least about 85% identity to SEQ ID NO: 13 and comprising the TLR7-selective motif of SEQ ID NO: 45.

23. The immunomodulatory composition of any one of claims 1, 2, 5, 8, 12, and 14-20, wherein the RNA ligand comprises the nucleotide sequence of SEQ ID NO: 14, or a nucleotide sequence having at least about 85% identity to SEQ ID NO: 14 and comprising the TLR7-selective motif of SEQ ID NO: 46.

24. The immunomodulatory composition of any one of claims 1, 3-4, 9, and 13-20, wherein the RNA ligand comprises the nucleotide sequence of SEQ ID NO: 15, or a nucleotide sequence having at least about 85% identity to SEQ ID NO: 15 and comprising the TLR7-selective motif of SEQ ID NO: 47.

25. An immunomodulatory composition that selectively activates TLR7, comprising an RNA ligand and a pharmaceutically acceptable carrier, wherein the RNA ligand comprises:
(a) a nucleotide sequence selected from the group consisting of SEQ ID NOs: 12, 13, 14, and 15;
(b) a nucleotide sequence having at least about 85% identity to SEQ ID NO: 12 and comprising the TLR7-selective motif of SEQ ID NO: 44;
(c) a nucleotide sequence having at least about 85% identity to SEQ ID NO: 13 and comprising the TLR7-selective motif of SEQ ID NO: 45;
(d) a nucleotide sequence having at least about 85% identity to SEQ ID NO: 14 and comprising the TLR7-selective motif of SEQ ID NO: 46; or
(e) a nucleotide sequence having at least about 85% identity to SEQ ID NO: 15 and comprising the TLR7-selective motif of SEQ ID NO: 47.

26. An immunomodulatory composition that selectively activates TLR7, comprising an RNA ligand and a pharmaceutically acceptable carrier, wherein the RNA ligand comprises:
(a) a TLR7-selective motif comprising Formula III:
C₁-U₂-C₃ (Formula III),
wherein C₁ and C₃ are cytidine nucleosides and U₂ is a uridine nucleoside,
wherein C₁ has a 2′O-methyl modification (mU), a 2′-fluoro modification (fU), a 2′-amino modification, a 2′-deoxy modification, or a 2′-methoxyethoxy modification, and
wherein “-” represents a phosphodiester linkage;
(b) a first nucleotide sequence linked to the 5′ end of the TLR7-selective motif; and
(c) a second nucleotide sequence linked to the 3′ end of the TLR7-selective motif.

27. The immunomodulatory composition of claim 26, wherein C₁ of the TLR7-selective motif has a 2′-fluoro modification (fC).

28. The immunomodulatory composition of claim 26, wherein C₁ of the TLR7-selective motif has a 2′O-methyl modification (mC).

29. The immunomodulatory composition of claim 26, wherein the TLR7-selective motif comprises the nucleotide sequence of SEQ ID NO: 42.

30. The immunomodulatory composition of claim 26, wherein the TLR7-selective motif comprises the nucleotide sequence of SEQ ID NO: 43.

31. An immunomodulatory composition that selectively activates TLR7, comprising an RNA ligand and a pharmaceutically acceptable carrier, wherein the RNA ligand comprises:
(a) a TLR7-selective motif comprising the nucleotide sequence of fC-U-C or mC-U-C, wherein “-” represents a phosphodiester linkage, “m” represents a 2′O-methyl modification, and “f” represents a 2′-fluoro modification,
(b) a first nucleotide sequence linked to the 5′ end of the TLR7-selective motif, and
(c) a second nucleotide sequence linked to the 3′ end of the TLR7-selective motif.

32. The immunomodulatory composition of any one of claims 26-31, wherein the RNA ligand is capable of adopting a double-stranded RNA hairpin structure.

33. The immunomodulatory composition of any one of claims 26-32, wherein the RNA ligand comprises a G:U wobble base pair.

34. The immunomodulatory composition of any one of claims 26-33, wherein the first nucleotide sequence does not comprise a uridine nucleoside, and
wherein the first nucleotide sequence is linked to the TLR7-selective motif by a phosphodiester linkage.

35. The immunomodulatory composition of claim 34, wherein the first nucleotide sequence comprises three nucleosides.

36. The immunomodulatory composition of claim 34 or claim 35, wherein the nucleosides within the first nucleotide sequence are linked by phosphodiester linkages.

37. The immunomodulatory composition of any one of claims 26-36, wherein the first nucleotide sequence comprises the nucleotide sequence C-G-G, wherein “-” represents a phosphodiester linkage.

38. The immunomodulatory composition of any one of claims 26-37, wherein the second nucleotide sequence does not comprise a uridine nucleoside,
wherein the second nucleotide sequence comprises the nucleotide sequence G-G-G,
wherein the nucleotide sequence G-G-G is capable of base pairing with the TLR7-selective motif, and
wherein the second nucleotide sequence is linked to the TLR7-selective motif by a phosphodiester linkage.

39. The immunomodulatory composition of claim 38, wherein the second nucleotide sequence comprises about 15 nucleosides.

40. The immunomodulatory composition of claim 38 or claim 39, wherein the nucleosides within the second nucleotide sequence are linked by phosphodiester linkages.

41. The immunomodulatory composition of any one of claims 26-40, wherein the second nucleotide sequence comprises:
(a) the nucleotide sequence G-G-C-A-G-A-A-G-C-C-G-G-G-C-C(SEQ ID NO: 61), or
(b) a nucleotide sequence having at least about 85% identity to SEQ ID NO: 61 and comprising the nucleotide sequence G-G-G, wherein the nucleotide sequence G-G-G is capable of base pairing with the TLR7-selective motif, and
wherein “-” represents a phosphodiester linkage.

42. The immunomodulatory composition of any one of claims 26-27, 29, and 31-41, wherein the RNA ligand comprises:
(a) the nucleotide sequence of SEQ ID NO: 23, or
(b) a nucleotide sequence having at least about 85% identity to SEQ ID NO: 23 and comprising the TLR7-selective motif of SEQ ID NO: 42 and the nucleotide sequence G-G-G, wherein the nucleotide sequence G-G-G is capable of base pairing with the TLR7-selective motif.

43. The immunomodulatory composition of any one of claims 26, 28, and 30-41, wherein the RNA ligand comprises:
(a) the nucleotide sequence of SEQ ID NO: 24, or
(b) a nucleotide sequence having at least about 85% identity to SEQ ID NO: 24 and comprising the TLR7-selective motif of SEQ ID NO: 43 and the nucleotide sequence G-G-G, wherein the nucleotide sequence G-G-G is capable of base pairing with the TLR7-selective motif.

44. An immunomodulatory composition that selectively activates TLR7, comprising an RNA ligand and a pharmaceutically acceptable carrier, wherein the RNA ligand comprises:
(a) a nucleotide sequence of SEQ ID NOs: 23 or 24;
(b) a nucleotide sequence having at least about 85% identity to SEQ ID NO: 23 and comprising the TLR7-selective motif of SEQ ID NO: 42 and the nucleotide sequence G-G-G, wherein the nucleotide sequence G-G-G is capable of base pairing with the TLR7-selective motif; or
(c) a nucleotide sequence having at least about 85% identity to SEQ ID NO: 24 and comprising the TLR7-selective motif of SEQ ID NO: 43 and the nucleotide sequence G-G-G, wherein the nucleotide sequence G-G-G is capable of base pairing with the TLR7-selective motif.

45. An immunomodulatory composition that selectively activates TLR7, comprising an RNA ligand and a pharmaceutically acceptable carrier, wherein the RNA ligand comprises:
(a) a first and a second TLR7-selective motif comprising Formula IV:
C₁-U₂-U₃-C₄ (Formula IV),
wherein C₁ and C₄ are cytidine nucleosides and U₂ and U₃ are uridine nucleosides,
wherein U₂ has a 2′O-methyl modification (mU), a 2′-fluoro modification (fU), a 2′-amino modification, a 2′-deoxy modification, or a 2′-methoxyethoxy modification, and
wherein “-” represents a phosphodiester linkage;
(b) a first nucleotide sequence linked to the 5′ end of the first TLR7-selective motif; and
(c) a second nucleotide sequence linked to the 3′ end of the first TLR7-selective motif and to the 5′ end of the second TLR7-selective motif.

46. The immunomodulatory composition of claim 45, wherein U₂ of the first TLR7-selective motif has a 2′O-methyl modification (mU).

47. The immunomodulatory composition of claim 45 or claim 46, wherein U₂ of the second TLR7-selective motif has a 2′O-methyl modification (mU).

48. The immunomodulatory composition of claim 45, wherein U₂ of the first TLR7-selective motif has a 2′-fluoro modification (RU).

49. The immunomodulatory composition of claim 45 or claim 48, wherein U₂ of the second TLR7-selective motif has a 2′-fluoro modification (fU).

50. The immunomodulatory composition of claim 45, wherein the first TLR7-selective motif comprises the nucleotide sequence of SEQ ID NO: 36.

51. The immunomodulatory composition of claim 45 or claim 50, wherein the second TLR7-selective motif comprises the nucleotide sequence of SEQ ID NO: 36.

52. The immunomodulatory composition of claim 45, wherein the first TLR7-selective motif comprises the nucleotide sequence of SEQ ID NO: 37.

53. The immunomodulatory composition of claim 45 or claim 52, wherein the second TLR7-selective motif comprises the nucleotide sequence of SEQ ID NO: 37.

54. An immunomodulatory composition that selectively activates TLR7, comprising an RNA ligand and a pharmaceutically acceptable carrier, wherein the RNA ligand comprises:
(a) a first TLR7-selective motif comprising the nucleotide sequence of SEQ ID NO: 36 or 37;
(b) a second TLR7-selective motif comprising the nucleotide sequence of SEQ ID NO: 36 or 37;
(c) a first nucleotide sequence linked to the 5′ end of the first TLR7-selective motif; and
(d) a second nucleotide sequence linked to the 3′ end of the first TLR7-selective motif and to the 5′ end of the second TLR7-selective motif.

55. The immunomodulatory composition of any one of claims 45-54, wherein the first nucleotide sequence comprises one nucleoside, or at least two nucleosides linked by phosphodiester linkages,
wherein the first nucleotide sequence does not comprise a uridine nucleoside, and
wherein the first nucleotide sequence is linked to the first TLR7-selective motif by a phosphodiester linkage.

56. The immunomodulatory composition of any one of claims 45-55, wherein the second nucleotide sequence comprises one nucleoside, or at least two nucleosides linked by phosphodiester linkages,
wherein the second nucleotide sequence does not comprise a uridine nucleoside, and
wherein the second nucleotide sequence is linked to the first TLR7-selective motif and/or to the second TLR7-selective motif by a phosphodiester linkage.

57. The immunomodulatory composition of claim 55 or claim 56, wherein the first nucleotide sequence and/or the second nucleotide sequence comprise five nucleosides linked by phosphodiester linkages.

58. The immunomodulatory composition of any one of claims 45-57, wherein the first nucleotide sequence and/or the second nucleotide sequence comprise the nucleotide sequence C-A-G-A-C, or a nucleotide sequence having at least about 85% identity to SEQ ID NO: 67, wherein “-” represents a phosphodiester linkage.

59. The immunomodulatory composition of any one of claims 45-47, 50-51, and 54-58, wherein the RNA ligand comprises the nucleotide sequence of SEQ ID NO: 33, or a nucleotide sequence having at least about 85% identity to SEQ ID NO: 33 and comprising the first TLR7-selective motif of SEQ ID NO: 36 and/or the second TLR7-selective motif of SEQ ID NO: 36.

60. The immunomodulatory composition of any one of claims 45, 48-49, and 52-58, wherein the RNA ligand comprises the nucleotide sequence of SEQ ID NO: 34, or a nucleotide sequence having at least about 85% identity to SEQ ID NO: 34 and comprising the first TLR7-selective motif of SEQ ID NO: 37 and/or the second TLR7-selective motif of SEQ ID NO: 37.

61. An immunomodulatory composition that selectively activates TLR7, comprising an RNA ligand and a pharmaceutically acceptable carrier, wherein the RNA ligand comprises:
(a) a nucleotide sequence of SEQ ID NOs: 33 or 34;
(b) a nucleotide sequence having at least about 85% identity to SEQ ID NO: 33 and comprising a first and a second TLR7-selective motif of SEQ ID NO: 36; or
(c) a nucleotide sequence having at least about 85% identity to SEQ ID NO: 34 and comprising a first and a second TLR7-selective motif of SEQ ID NO: 37.

62. An immunomodulatory composition that selectively activates TLR7, comprising an RNA ligand and a pharmaceutically acceptable carrier, wherein the RNA ligand comprises:
(a) a first TLR7-selective motif and one or more additional TLR7-selective motifs comprising Formula V:
U₁-U₂-C₃ (Formula V),
wherein U₁ and U₂ are uridine nucleosides and C₃ is a cytidine nucleoside,
wherein U₁ has a 2′O-methyl modification (mU), a 2′-fluoro modification (fU), a 2′-amino modification, a 2′-deoxy modification, or a 2′-methoxyethoxy modification, and
wherein “-” represents a phosphodiester linkage; and
(b) a first nucleotide sequence linked to the 5′ end of the first TLR7-selective motif.

63. The immunomodulatory composition of claim 62, wherein the one or more additional TLR7-selective motifs comprise one, two, three, four, five, or more additional TLR7-selective motifs comprising Formula V.

64. The immunomodulatory composition of claim 62 or claim 63, wherein the one or more additional TLR7-selective motifs comprise five additional TLR7-selective motifs comprising Formula V.

65. The immunomodulatory composition of any one of claims 62-64, wherein U₁ of the first TLR7-selective motif has a 2′O-methyl modification (mU).

66. The immunomodulatory composition of any one of claims 62-65, wherein U₁ of at least one of the one or more additional TLR7-selective motifs, or U₁ of all of the one or more additional TLR7-selective motifs, has a 2′O-methyl modification (mU).

67. The immunomodulatory composition of any one of claims 62-64, wherein U₁ of the first TLR7-selective motif has a 2′-fluoro modification.

68. The immunomodulatory composition of any one of claims 62-64 and 67, wherein U₁ of at least one of the one or more additional TLR7-selective motifs, or U₁ of all of the one or more additional TLR7-selective motifs, has a 2′-fluoro modification.

69. The immunomodulatory composition of any one of claims 62-64, wherein the first TLR7-selective motif comprises the nucleotide sequence of SEQ ID NO: 40.

70. The immunomodulatory composition of any one of claims 62-64 and 69, wherein at least one of the one or more additional TLR7-selective motifs, or all of the one or more additional TLR7-selective motifs comprise the nucleotide sequence of SEQ ID NO: 40.

71. The immunomodulatory composition of any one of claims 62-64, wherein the first TLR7-selective motif comprises the nucleotide sequence of SEQ ID NO: 41.

72. The immunomodulatory composition of any one of claims 62-64 and 71, wherein at least one of the one or more additional TLR7-selective motifs, or all of the one or more additional TLR7-selective motifs comprise the nucleotide sequence of SEQ ID NO: 41.

73. An immunomodulatory composition that selectively activates TLR7, comprising an RNA ligand and a pharmaceutically acceptable carrier, wherein the RNA ligand comprises:
(a) a first TLR7-selective motif comprising the nucleotide sequence of SEQ ID NO: 40 or 41;
(b) one or more additional TLR7-selective motifs, wherein at least one of the one or more additional TLR7-selective motifs, or all of the one or more additional TLR7-selective motifs comprise the nucleotide sequence of SEQ ID NO: 40 or 41; and
(c) a first nucleotide sequence linked to the 5′ end of the first TLR7-selective motif.

74. The immunomodulatory composition of claim 73, wherein the one or more additional TLR7-selective motifs comprise one, two, three, four, five, or more additional TLR7-selective motifs comprising the nucleotide sequence of SEQ ID NO: 40 or 41.

75. The immunomodulatory composition of claim 73 or claim 74, wherein the one or more additional TLR7-selective motifs comprise five additional TLR7-selective motifs comprising the nucleotide sequence of SEQ ID NO: 40 or 41.

76. The immunomodulatory composition of any one of claims 62-75, wherein the first nucleotide sequence comprises one nucleoside, or at least two nucleosides linked by phosphodiester linkages,
wherein the first nucleotide sequence does not comprise a uridine nucleoside, and
wherein the first nucleotide sequence is linked to the first TLR7-selective motif by a phosphodiester linkage.

77. The immunomodulatory composition of claim 76, wherein the first nucleotide sequence comprises two nucleosides linked by phosphodiester linkages.

78. The immunomodulatory composition of claim 76 or claim 77, wherein the first nucleotide sequence comprises two cytidine nucleosides linked by phosphodiester linkages.

79. The immunomodulatory composition of any one of claims 62-66, 69-70, and 73-78, wherein the RNA ligand comprises the nucleotide sequence of SEQ ID NO: 29, or a nucleotide sequence having at least about 85% identity to SEQ ID NO: 29 and comprising the first TLR7-selective motif of SEQ ID NO: 40 and five additional TLR7-selective motifs of SEQ ID NO: 40.

80. The immunomodulatory composition of any one of claims 62-64, 67-68, and 71-78, wherein the RNA ligand comprises the nucleotide sequence of SEQ ID NO: 30, or a nucleotide sequence having at least about 85% identity to SEQ ID NO: 30 and comprising the first TLR7-selective motif of SEQ ID NO: 41 and five additional TLR7-selective motifs of SEQ ID NO: 41.

81. An immunomodulatory composition that selectively activates TLR7, comprising an RNA ligand and a pharmaceutically acceptable carrier, wherein the RNA ligand comprises:
(a) a nucleotide sequence of SEQ ID NOs: 29 or 30;
(b) a nucleotide sequence having at least about 85% identity to SEQ ID NO: 29 and comprising six TLR7-selective motifs of SEQ ID NO: 40; or
(c) a nucleotide sequence having at least about 85% identity to SEQ ID NO: 30 and comprising six TLR7-selective motifs of SEQ ID NO: 41.

82. An immunomodulatory composition that selectively activates TLR7, comprising an RNA ligand and a pharmaceutically acceptable carrier, wherein the RNA ligand comprises:
(a) a TLR7-selective motif comprising Formula VI:
C₁-U₂-U₃*U₄-U₅*C₆ (Formula VI),
wherein C₁ and C₆ are cytidine nucleosides, and U₂, U₃, U₄, and U₅ are uridine nucleosides,
wherein U₂ has a 2′O-methyl modification (mU), a 2′-fluoro modification (fU), a 2′-amino modification, a 2′-deoxy modification, or a 2′-methoxyethoxy modification,
wherein U₄ has a 2′O-methyl modification (mU), a 2′-fluoro modification (fU), a 2′-amino modification, a 2′-deoxy modification, or a 2′-methoxyethoxy modification, and
wherein “*” represents a phosphorothioate linkage and “-” represents a phosphodiester linkage;
(b) a first nucleotide sequence linked to the 5′ end of the TLR7-selective motif; and
(c) a second nucleotide sequence linked to the 3′ end of the TLR7-selective motif.

83. The immunomodulatory composition of claim 82, wherein U₂ of the TLR7-selective motif has a 2′O-methyl modification (mU).

84. The immunomodulatory composition of claim 82, wherein U₄ of the TLR7-selective motif has a 2′O-methyl modification (mU).

85. The immunomodulatory composition of claim 82, wherein U₂ of the TLR7-selective motif has a 2′-fluoro modification (RU).

86. The immunomodulatory composition of claim 82, wherein U₄ of the TLR7-selective motif has a 2′-fluoro modification (RU).

87. The immunomodulatory composition of any one of claims 82-84, wherein U₂ and U₄ of the TLR7-selective motif have a 2′O-methyl modification (mU).

88. The immunomodulatory composition of any one of claims 82 and 85-86, wherein U₂ and U₄ of the TLR7-selective motif have a 2′-fluoro modification (fU).

89. The immunomodulatory composition of any one of claims 82-83 and 86, wherein U₂ of the TLR7-selective motif has a 2′O-methyl modification (mU), and U₄ of the TLR7-selective motif has a 2′-fluoro modification (RI).

90. The immunomodulatory composition of any one of claims 82 and 84-85, wherein U₂ of the TLR7-selective motif has a 2′-fluoro modification (RI), and U₄ of the TLR7-selective motif has a 2′O-methyl modification (mU).

91. The immunomodulatory composition of any one of claims 82-84, wherein the TLR7-selective motif comprises the nucleotide sequence of SEQ ID NO: 48.

92. The immunomodulatory composition of any one of claims 82 and 85-86, wherein the TLR7-selective motif comprises the nucleotide sequence of SEQ ID NO: 49.

93. The immunomodulatory composition of any one of claims 82-83 and 86, wherein the TLR7-selective motif comprises the nucleotide sequence of SEQ ID NO: 50.

94. The immunomodulatory composition of any one of claims 82 and 84-85, wherein the TLR7-selective motif comprises the nucleotide sequence of SEQ ID NO: 51.

95. An immunomodulatory composition that selectively activates TLR7, comprising an RNA ligand and a pharmaceutically acceptable carrier, wherein the RNA ligand comprises:
(a) a TLR7-selective motif comprising a nucleotide sequence selected from the group consisting of: C-mU-U*mU-U*C, C-fU-U*fU-U*C, C-mU-U*fU-U*C, and C-fU-U*mU-U*C, wherein “*” represents a phosphorothioate linkage, “-” represents a phosphodiester linkage, “m” indicates a 2′O-methyl modification, and “f” indicates a 2′-fluoro modification;
(b) a first nucleotide sequence linked to the 5′ end of the TLR7-selective motif; and
(c) a second nucleotide sequence linked to the 3′ end of the TLR7-selective motif.

96. The immunomodulatory composition of any one of claims 82-95, wherein the first nucleotide sequence comprises one nucleoside, or at least two nucleosides linked by phosphodiester linkages,
wherein the first nucleotide sequence does not comprise a uridine nucleoside, and
wherein the first nucleotide sequence is linked to the TLR7-selective motif by a phosphodiester linkage.

97. The immunomodulatory composition of claim 96, wherein the first nucleotide sequence comprises eight nucleosides linked by phosphodiester linkages.

98. The immunomodulatory composition of any one of claims 82-97, wherein the first nucleotide sequence comprises the nucleotide sequence C-C-G-A-G-C-C-G, or a nucleotide sequence having at least about 85% identity to SEQ ID NO: 68,
wherein “-” represents a phosphodiester linkage, and
wherein the first nucleotide sequence is linked to the TLR7-selective motif by a phosphodiester linkage.

99. The immunomodulatory composition of any one of claims 82-98, wherein the second nucleotide sequence comprises one nucleoside, or at least two nucleosides linked by phosphodiester linkages,
wherein the second nucleotide sequence does not comprise a uridine nucleoside, and
wherein the second nucleotide sequence is linked to the TLR7-selective motif by a phosphodiester linkage.

100. The immunomodulatory composition of claim 99, wherein the second nucleotide sequence comprises two nucleosides linked by phosphodiester linkages.

101. The immunomodulatory composition of claim 100, wherein the second nucleotide sequence comprises two cytidine nucleosides linked by phosphodiester linkages.

102. The immunomodulatory composition of any one of claims 82-84, 87, 91, and 95-101, wherein the RNA ligand comprises the nucleotide sequence of SEQ ID NO: 17, or a nucleotide sequence having at least about 85% identity to SEQ ID NO: 17 and comprising the TLR7-selective motif of SEQ ID NO: 48.

103. The immunomodulatory composition of any one of claims 82, 85-86, 88, 92, and 95-101, wherein the RNA ligand comprises the nucleotide sequence of SEQ ID NO: 18, or a nucleotide sequence having at least about 85% identity to SEQ ID NO: 18 and comprising the TLR7-selective motif of SEQ ID NO: 49.

104. The immunomodulatory composition of any one of claims 82, 83, 86, 89, 93, and 95-101, wherein the RNA ligand comprises the nucleotide sequence of SEQ ID NO: 19, or a nucleotide sequence having at least about 85% identity to SEQ ID NO: 19 and comprising the TLR7-selective motif of SEQ ID NO: 50.

105. The immunomodulatory composition of any one of claims 82, 84-85, 90, and 94-101, wherein the RNA ligand comprises the nucleotide sequence of SEQ ID NO: 20, or a nucleotide sequence having at least about 85% identity to SEQ ID NO: 20 and comprising the TLR7-selective motif of SEQ ID NO: 51.

106. An immunomodulatory composition that selectively activates TLR7, comprising an RNA ligand and a pharmaceutically acceptable carrier, wherein the RNA ligand comprises:
(a) a nucleotide sequence selected from the group consisting of SEQ ID NOs: 17, 18, 19, and 20;
(b) a nucleotide sequence having at least about 85% identity to SEQ ID NO: 17 and comprising the TLR7-selective motif of SEQ ID NO: 48;
(c) a nucleotide sequence having at least about 85% identity to SEQ ID NO: 18 and comprising the TLR7-selective motif of SEQ ID NO: 49;
(d) a nucleotide sequence having at least about 85% identity to SEQ ID NO: 19 and comprising the TLR7-selective motif of SEQ ID NO: 50; or
(e) a nucleotide sequence having at least about 85% identity to SEQ ID NO: 20 and comprising the TLR7-selective motif of SEQ ID NO: 51.

107. The immunomodulatory composition of any one of claims 1-106, wherein the pharmaceutically acceptable carrier is a lipid nanoparticle (LNP).

108. The immunomodulatory composition of claim 107, wherein the LNP comprises a cationic lipid.

109. The immunomodulatory composition of claim 107 or claim 108, wherein the LNP comprises an ionizable lipid.

110. The immunomodulatory composition of any one of claims 1-109, wherein the pharmaceutically acceptable carrier is or comprises poly-L-arginine or 1,2-Dioleoyl-3-trimethylammonium propane (DOTAP).

111. The immunomodulatory composition of any one of claims 1-110, wherein the immunomodulatory composition selectively activates TLR7 in one or more cells contacted with the immunomodulatory composition.

112. The immunomodulatory composition of claim 111, wherein the one or more cells express TLR7.

113. The immunomodulatory composition of claim 111 or claim 112, wherein the one or more cells comprise peripheral blood mononuclear cells (PBMCs).

114. The immunomodulatory composition of any one of claims 111-113, wherein the one or more cells comprise one or more plasmacytoid dendritic cells.

115. The immunomodulatory composition of any one of claims 1-114, wherein the immunomodulatory composition increases TLR7 activity in one or more cells contacted with the immunomodulatory composition, as compared to corresponding one or more cells that are not contacted with the immunomodulatory composition.

116. The immunomodulatory composition of any one of claims 1-115, wherein the immunomodulatory composition increases secretion of IFN-α by one or more cells contacted with the immunomodulatory composition, as compared to corresponding one or more cells that are not contacted with the immunomodulatory composition.

117. The immunomodulatory composition of claim 115, wherein the increase in TLR7 activity in the one or more cells contacted with the immunomodulatory composition is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition.

118. The immunomodulatory composition of claim 116, wherein the increase in secretion of IFN-α by the one or more cells contacted with the immunomodulatory composition is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition.

119. The immunomodulatory composition of any one of claims 1-118, wherein the immunomodulatory composition increases TLR8 activity in one or more cells contacted with the immunomodulatory composition by less than about 20%, less than about 15%, less than about 10%, less than about 5%, or less than about 1%, as compared to corresponding one or more cells that are not contacted with the immunomodulatory composition.

120. The immunomodulatory composition of any one of claims 1-119, wherein the immunomodulatory composition increases secretion of TNF-α by one or more cells contacted with the immunomodulatory composition by less than about 20%, less than about 15%, less than about 10%, less than about 5%, or less than about 1%, as compared to corresponding one or more cells that are not contacted with the immunomodulatory composition.

121. The immunomodulatory composition of any one of claims 111-120, wherein the RNA ligand is introduced into the one or more cells contacted with the immunomodulatory composition.

122. The immunomodulatory composition of any one of claims 111-121, wherein the one or more cells contacted with the immunomodulatory composition are further contacted with guanosine or a guanosine derivative, optionally wherein the guanosine derivative is 2′3′ cyclic GMP.

123. An isolated nucleic acid comprising the RNA ligand of the immunomodulatory composition of any one of claims 1-122.

124. The nucleic acid of claim 123, wherein the nucleic acid is a DNA, RNA or a DNA/RNA molecule.

125. A vector comprising the RNA ligand of the immunomodulatory composition of any one of claims 1-122 or the nucleic acid of claim 123 or claim 124.

126. A pharmaceutical composition comprising the immunomodulatory composition of any one of claims 1-122.

127. A host cell comprising the RNA ligand of the immunomodulatory composition of any one of claims 1-122, the nucleic acid of claim 123 or claim 124, or the vector of claim 125.

128. A vaccine composition comprising the immunomodulatory composition of any one of claims 1-122 or the pharmaceutical composition of claim 126.

129. A method for selectively activating TLR7 in one or more cells, the method comprising contacting one or more cells with the immunomodulatory composition of any one of claims 1-122, or the pharmaceutical composition of claim 126.

130. The method of claim 129, wherein the one or more cells express TLR7.

131. The method of claim 129 or claim 130, wherein the one or more cells comprise peripheral blood mononuclear cells (PBMCs).

132. The method of any one of claims 129-131, wherein the one or more cells comprise one or more plasmacytoid dendritic cells.

133. The method of any one of claims 129-132, wherein contacting the one or more cells with the immunomodulatory composition of any one of claims 1-122, or the pharmaceutical composition of claim 126 results in an increase in TLR7 activity in the one or more cells, as compared to corresponding one or more cells that are not contacted with the immunomodulatory composition, or the pharmaceutical composition.

134. The method of any one of claims 129-133, wherein contacting the one or more cells with the immunomodulatory composition of any one of claims 1-122, or the pharmaceutical composition of claim 126 results in an increase in secretion by the one or more cells of IFN-α, as compared to corresponding one or more cells that are not contacted with the immunomodulatory composition, or the pharmaceutical composition.

135. The method of claim 133, wherein the increase in TLR7 activity in the one or more cells is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition, or the pharmaceutical composition.

136. The method of claim 134, wherein the increase in secretion by the one or more cells of IFN-α is an increase of at least about 5%, at least about 10%, at least about 20%, at least about 30%, at least about 40%, at least about 50%, at least about 60%, at least about 70%, at least about 80%, at least about 90%, at least about 100%, or more, as compared to corresponding one or more cells that are not contacted with the immunomodulatory composition, or the pharmaceutical composition.

137. The method of any one of claims 129-136, wherein contacting the one or more cells with the immunomodulatory composition of any one of claims 1-122, or the pharmaceutical composition of claim 126 results in an increase in TLR8 activity in the one or more cells of less than about 20%, less than about 15%, less than about 10%, less than about 5%, or less than about 1%, as compared to corresponding one or more cells that are not contacted with the immunomodulatory composition, or the pharmaceutical composition.

138. The method of any one of claims 129-137, wherein contacting the one or more cells with the immunomodulatory composition of any one of claims 1-122, or the pharmaceutical composition of claim 126 results in an increase in secretion by the one or more cells of TNF-α of less than about 20%, less than about 15%, less than about 10%, less than about 5%, or less than about 1%, as compared to corresponding one or more cells that are not contacted with the immunomodulatory composition, or the pharmaceutical composition.

139. The method of any one of claims 129-138, further comprising contacting the one or more cells with guanosine or a guanosine derivative, optionally wherein the guanosine derivative is 2′3′ cyclic GMP.

140. The method of any one of claims 134-139, further comprising measuring secretion of IFN-α and/or TNF-α by enzyme-linked immunosorbent assay (ELISA), immunoblotting, immunoassays, flow cytometry, electrochemiluminescence-based methods, mass spectrometry, or qPCR; and/or measuring the expression levels of interferon-stimulated genes (ISGs).

141. The method of any one of claims 129-140, wherein contacting the one or more cells with the immunomodulatory composition of any one of claims 1-122, or the pharmaceutical composition of claim 126 comprises introducing the RNA ligand into the one or more cells.

142. A method for selectively activating TLR7 in an individual, the method comprising administering to the individual an effective amount of the immunomodulatory composition of any one of claims 1-122, the pharmaceutical composition of claim 126, or the vaccine composition of claim 128.

143. A method for stimulating an immune response in an individual, the method comprising administering to the individual an effective amount of the immunomodulatory composition of any one of claims 1-122, the pharmaceutical composition of claim 126, or the vaccine composition of claim 128.

144. The method of claim 142 or claim 143, wherein administering to the individual an effective amount of the immunomodulatory composition of any one of claims 1-122, the pharmaceutical composition of claim 126, or the vaccine composition of claim 128 results in an increase in TLR7 activity in the individual, as compared to a corresponding individual not administered the immunomodulatory composition, the pharmaceutical composition, or the vaccine composition.

145. The method of any one of claims 142-144, wherein administering to the individual an effective amount of the immunomodulatory composition of any one of claims 1-122, the pharmaceutical composition of claim 126, or the vaccine composition of claim 128 results in an increase in the individual of the levels of IFN-α, as compared to a corresponding individual not administered the immunomodulatory composition, the pharmaceutical composition, or the vaccine composition.

146. The method of claim 144, wherein the increase in TLR7 activity is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition.

147. The method of claim 145, wherein the increase of IFN-α levels is an increase of at least about 5%, at least about 10%, at least about 20%, at least about 30%, at least about 40%, at least about 50%, at least about 60%, at least about 70%, at least about 80%, at least about 90%, at least about 100%, or more, as compared to a corresponding individual not administered the immunomodulatory composition, the pharmaceutical composition, or the vaccine composition.

148. The method of any one of claims 142-147, wherein administering to the individual an effective amount of the immunomodulatory composition of any one of claims 1-122, the pharmaceutical composition of claim 126, or the vaccine composition of claim 128 results in an increase in TLR8 activity in the individual of less than about 20%, less than about 15%, less than about 10%, less than about 5%, or less than about 1%, as compared to a corresponding individual not administered the immunomodulatory composition, the pharmaceutical composition, or the vaccine composition.

149. The method of any one of claims 142-148, wherein administering to the individual an effective amount of the immunomodulatory composition of any one of claims 1-122, the pharmaceutical composition of claim 126, or the vaccine composition of claim 128 results in an increase in levels of TNF-α in the individual of less than about 20%, less than about 15%, less than about 10%, less than about 5%, or less than about 1%, as compared to a corresponding individual not administered the immunomodulatory composition, the pharmaceutical composition, or the vaccine composition.

150. An adjuvant for use in the manufacture of a medicament for activating TLR7 in an individual, wherein the adjuvant comprises the immunomodulatory composition of any one of claims 1-122, the pharmaceutical composition of claim 126, or the vaccine composition of claim 128.

151. An adjuvant for use in a method for activating TLR7 in an individual, the method comprising administering to the individual an effective amount of the immunomodulatory composition of any one of claims 1-122, the pharmaceutical composition of claim 126, or the vaccine composition of claim 128.

152. A kit comprising the immunomodulatory composition of any one of claims 1-122, the nucleic acid of claim 123 or claim 124, the vector of claim 125, the pharmaceutical composition of claim 126, or the vaccine composition of claim 128.


SEQUENCE LISTING (72 sequences)

SEQ ID NO: 1 - RNA, 16 nt, synthetic construct
  misc_feature 1..16: note = Modified RNA
  source 1..16: mol_type = other RNA
  modified_base 1..16: mod_base = OTHER; note = phosphorothioate linkage between nucleosides
ccgagccgcttttccc

SEQ ID NO: 2 - RNA, 21 nt, synthetic construct
  misc_feature 1..21: note = Modified RNA
  source 1..21: mol_type = other RNA
  modified_base 1..21: mod_base = OTHER; note = phosphodiester linkage between nucleosides
cggctcggcagaagccgggcc

SEQ ID NO: 3 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
tctctctctctctctctctc

SEQ ID NO: 4 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
ccttcttcttcttcttcttc

SEQ ID NO: 5 - RNA, 18 nt, synthetic construct
  misc_feature 1..18: note = Modified RNA
  source 1..18: mol_type = other RNA
  modified_base 1..18: mod_base = OTHER; note = phosphodiester linkage between nucleosides
cagaccttccagaccttc

SEQ ID NO: 6-11 - skipped (6 entries)

SEQ ID NO: 12 - RNA, 16 nt, synthetic construct
  misc_feature 1..16: note = Modified RNA
  source 1..16: mol_type = other RNA
  modified_base 10: mod_base = OTHER; note = 2'O-methyl modification
  modified_base 12: mod_base = OTHER; note = 2'O-methyl modification
  modified_base 1..10: mod_base = OTHER; note = phosphorothioate linkage between nucleosides
  modified_base 11..12: mod_base = OTHER; note = phosphorothioate linkage between nucleosides
  modified_base 10..11: mod_base = OTHER; note = phosphodiester linkage between nucleosides
  modified_base 12..13: mod_base = OTHER; note = phosphodiester linkage between nucleosides
  modified_base 13..16: mod_base = OTHER; note = phosphorothioate linkage between nucleosides
ccgagccgcttttccc

SEQ ID NO: 13 - RNA, 16 nt, synthetic construct
  misc_feature 1..16: note = Modified RNA
  source 1..16: mol_type = other RNA
  modified_base 10: mod_base = OTHER; note = 2'-fluoro modification
  modified_base 12: mod_base = OTHER; note = 2'-fluoro modification
  modified_base 1..10: mod_base = OTHER; note = phosphorothioate linkage between nucleosides
  modified_base 10..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..16: mod_base = OTHER; note = phosphorothioate linkage between nucleosides
ccgagccgcttttccc

SEQ ID NO: 14 - RNA, 16 nt, synthetic construct
  misc_feature 1..16: note = Modified RNA
  source 1..16: mol_type = other RNA
  modified_base 1..10: mod_base = OTHER; note = phosphorothioate linkage between nucleosides
  modified_base 10..11: mod_base = OTHER; note = phosphodiester linkage between nucleosides
  modified_base 11..12: mod_base = OTHER; note = phosphodiester linkage between nucleosides
  modified_base 12..13: mod_base = OTHER; note = phosphodiester linkage between nucleosides
  modified_base 13..16: mod_base = OTHER; note = phosphorothioate linkage between nucleosides
  modified_base 10: mod_base = OTHER; note = 2'O-methyl modification
  modified_base 12: mod_base = OTHER; note = 2'-fluoro modification
ccgagccgcttttccc

SEQ ID NO: 15 - RNA, 16 nt, synthetic construct
  misc_feature 1..16: note = Modified RNA
  source 1..16: mol_type = other RNA
  modified_base 1..10: mod_base = OTHER; note = phosphorothioate linkage between nucleosides
  modified_base 10..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..16: mod_base = OTHER; note = phosphorothioate linkage between nucleosides
  modified_base 10: mod_base = OTHER; note = 2'-fluoro modification
  modified_base 12: mod_base = OTHER; note = 2'O-methyl modification
ccgagccgcttttccc

SEQ ID NO: 16 - RNA, 16 nt, synthetic construct
  misc_feature 1..16: note = Modified RNA
  source 1..16: mol_type = other RNA
  modified_base 1..9: mod_base = OTHER; note = phosphodiester linkage between nucleosides
  modified_base 9..14: mod_base = OTHER; note = phosphorothioate linkage between nucleosides
  modified_base 14..16: mod_base = OTHER; note = phosphodiester linkage between nucleosides
ccgagccgcttttccc

SEQ ID NO: 17 - RNA, 16 nt, synthetic construct
  misc_feature 1..16: note = Modified RNA
  source 1..16: mol_type = other RNA
  modified_base 1..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..14: mod_base = OTHER; note = phosphorothioate linkage between nucleosides
  modified_base 14..16: mod_base = OTHER; note = phosphodiester linkage between nucleosides
  modified_base 10: mod_base = OTHER; note = 2'O-methyl modification
  modified_base 12: mod_base = OTHER; note = 2'O-methyl modification
ccgagccgcttttccc

SEQ ID NO: 18 - RNA, 16 nt, synthetic construct
  misc_feature 1..16: note = Modified RNA
  source 1..16: mol_type = other RNA
  modified_base 1..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..14: mod_base = OTHER; note = phosphorothioate linkage between nucleosides
  modified_base 14..16: mod_base = OTHER; note = phosphodiester linkage between nucleosides
  modified_base 10: mod_base = OTHER; note = 2'O-methyl modification
  modified_base 12: mod_base = OTHER; note = 2'-fluoro modification
ccgagccgcttttccc

SEQ ID NO: 19 - RNA, 16 nt, synthetic construct
  misc_feature 1..16: note = Modified RNA
  source 1..16: mol_type = other RNA
  modified_base 1..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..14: mod_base = OTHER; note = phosphorothioate linkage between nucleosides
  modified_base 14..16: mod_base = OTHER; note = phosphodiester linkage between nucleosides
  modified_base 10: mod_base = OTHER; note = 2'O-methyl modification
  modified_base 12: mod_base = OTHER; note = 2'-fluoro modification
ccgagccgcttttccc

SEQ ID NO: 20 - RNA, 16 nt, synthetic construct
  misc_feature 1..16: note = Modified RNA
  source 1..16: mol_type = other RNA
  modified_base 1..11: mod_base = OTHER; note = phosphodiester linkage between nucleosides
  modified_base 11..12: mod_base = OTHER; note = phosphorothioate linkage between nucleosides
  modified_base 12..13: mod_base = OTHER; note = phosphodiester linkage between nucleosides
  modified_base 13..14: mod_base = OTHER; note = phosphorothioate linkage between nucleosides
  modified_base 14..16: mod_base = OTHER; note = phosphodiester linkage between nucleosides
  modified_base 10: mod_base = OTHER; note = 2'-fluoro modification
  modified_base 12: mod_base = OTHER; note = 2'O-methyl modification
ccgagccgcttttccc

SEQ ID NO: 21-22 - skipped (2 entries)

SEQ ID NO: 23 - RNA, 21 nt, synthetic construct
  misc_feature 1..21: note = Modified RNA
  source 1..21: mol_type = other RNA
  modified_base 1..21: mod_base = OTHER; note = phosphodiester linkage between nucleosides
  modified_base 4: mod_base = OTHER; note = 2'-fluoro modification
cggctcggcagaagccgggcc

SEQ ID NO: 24 - RNA, 21 nt, synthetic construct
  misc_feature 1..21: note = Modified RNA
  source 1..21: mol_type = other RNA
  modified_base 1..21: mod_base = OTHER; note = phosphodiester linkage between nucleosides
  modified_base 4: mod_base = OTHER; note = 2'O-methyl modification
cggctcggcagaagccgggcc

SEQ ID NO: 25 - RNA, 21 nt, synthetic construct
  misc_feature 1..21: note = Modified RNA
  source 1..21: mol_type = other RNA
  modified_base 1..21: mod_base = OTHER; note = phosphorothioate linkage between nucleosides
cggctcggcagaagccgggcc

SEQ ID NO: 26 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
  modified_base 2: mod_base = OTHER; note = 2'O-methyl modification
  modified_base 4: mod_base = OTHER; note = 2'O-methyl modification
  modified_base 6: mod_base = OTHER; note = 2'O-methyl modification
  modified_base 8: mod_base = OTHER; note = 2'O-methyl modification
  modified_base 10: mod_base = OTHER; note = 2'O-methyl modification
  modified_base 12: mod_base = OTHER; note = 2'O-methyl modification
  modified_base 14: mod_base = OTHER; note = 2'O-methyl modification
  modified_base 16: mod_base = OTHER; note = 2'O-methyl modification
  modified_base 18: mod_base = OTHER; note = 2'O-methyl modification
tctctctctctctctctctc

SEQ ID NO: 27 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
  modified_base 2: mod_base = OTHER; note = 2'-fluoro modification
  modified_base 4: mod_base = OTHER; note = 2'-fluoro modification
  modified_base 6: mod_base = OTHER; note = 2'-fluoro modification
  modified_base 8: mod_base = OTHER; note = 2'-fluoro modification
  modified_base 10: mod_base = OTHER; note = 2'-fluoro modification
  modified_base 12: mod_base = OTHER; note = 2'-fluoro modification
  modified_base 14: mod_base = OTHER; note = 2'-fluoro modification
  modified_base 16: mod_base = OTHER; note = 2'-fluoro modification
  modified_base 18: mod_base = OTHER; note = 2'-fluoro modification
tctctctctctctctctctc

SEQ ID NO: 28 - RNA, 20 nt, synthetic construct
  misc_feature 1..20: note = Modified RNA
  source 1..20: mol_type = other RNA
  modified_base 1..20: mod_base = OTHER; note = phosphorothioate linkage between nucleosides
tctctctctctctctctctc

SEQ ID NO: 29 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
  modified_base 3: mod_base = OTHER; note = 2'O-methyl modification
  modified_base 6: mod_base = OTHER; note = 2'O-methyl modification
  modified_base 9: mod_base = OTHER; note = 2'O-methyl modification
  modified_base 12: mod_base = OTHER; note = 2'O-methyl modification
  modified_base 15: mod_base = OTHER; note = 2'O-methyl modification
  modified_base 18: mod_base = OTHER; note = 2'O-methyl modification
ccttcttcttcttcttcttc

SEQ ID NO: 30 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
  modified_base 3: mod_base = OTHER; note = 2'-fluoro modification
  modified_base 6: mod_base = OTHER; note = 2'-fluoro modification
  modified_base 9: mod_base = OTHER; note = 2'-fluoro modification
  modified_base 12: mod_base = OTHER; note = 2'-fluoro modification
  modified_base 15: mod_base = OTHER; note = 2'-fluoro modification
  modified_base 18: mod_base = OTHER; note = 2'-fluoro modification
ccttcttcttcttcttcttc

SEQ ID NO: 31 - RNA, 20 nt, synthetic construct
  misc_feature 1..20: note = Modified RNA
  source 1..20: mol_type = other RNA
  modified_base 1..20: mod_base = OTHER; note = phosphorothioate linkage between nucleosides
ccttcttcttcttcttcttc

SEQ ID NO: 32 - skipped

SEQ ID NO: 33 - RNA, 18 nt, synthetic construct
  misc_feature 1..18: note = Modified RNA
  source 1..18: mol_type = other RNA
  modified_base 1..18: mod_base = OTHER; note = phosphodiester linkage between nucleosides
  modified_base 7: mod_base = OTHER; note = 2'O-methyl modification
  modified_base 16: mod_base = OTHER; note = 2'O-methyl modification
cagaccttccagaccttc

SEQ ID NO: 34 - RNA, 18 nt, synthetic construct
  misc_feature 1..18: note = Modified RNA
  source 1..18: mol_type = other RNA
  modified_base 1..18: mod_base = OTHER; note = phosphodiester linkage between nucleosides
  modified_base 7: mod_base = OTHER; note = 2'-fluoro modification
  modified_base 16: mod_base = OTHER; note = 2'-fluoro modification
cagaccttccagaccttc

SEQ ID NO: 35 - RNA, 18 nt, synthetic construct
  source 1..18: mol_type = other RNA; note = Modified RNA
  modified_base 1..6: mod_base = OTHER; note = phosphodiester linkage between nucleosides
  modified_base 6..9: mod_base = OTHER; note = phosphorothioate linkage between nucleosides
  modified_base 12..15: mod_base = OTHER; note = phosphodiester linkage between nucleosides
  modified_base 15..18: mod_base = OTHER; note = phosphorothioate linkage between nucleosides
cagaccttccagaccttc

SEQ ID NO: 36-55 - skipped (20 entries)

SEQ ID NO: 56 - RNA, 19 nt, synthetic construct
  misc_feature 1..19: note = Modified RNA
  source 1..19: mol_type = other RNA
  modified_base 1..19: mod_base = OTHER; note = phosphodiester linkage between nucleosides
agcttaacctgtccttcaa

SEQ ID NO: 57-60 - skipped (4 entries)

SEQ ID NO: 61 - DNA, 15 bp, synthetic construct
  misc_feature 1..15: note = Modified RNA
  source 1..15: mol_type = other DNA
  modified_base 1..15: mod_base = OTHER; note = phosphodiester linkage between nucleosides
ggcagaagccgggcc

SEQ ID NO: 62 - RNA, 55 nt, synthetic construct
  misc_feature 1..55: note = Modified RNA
  source 1..55: mol_type = other RNA
  modified_base 1..55: mod_base = OTHER; note = phosphodiester linkage between nucleosides
gggacgctgacccagaagatcactagaaatagtagatcttctgggtcagcgtccc

SEQ ID NO: 63-64 - skipped (2 entries)

SEQ ID NO: 65 - DNA, 76 bp, synthetic construct
  misc_feature 1..76: note = Modified RNA
  source 1..76: mol_type = other DNA
  modified_base 1..76: mod_base = OTHER; note = phosphodiester linkage between nucleosides
ttgtaatacgactcactatagggacgctgacccagaagatctactagaaatagtagatcttctgggtcagcgtccc

SEQ ID NO: 66 - DNA, 76 bp, synthetic construct
  misc_feature 1..76: note = Modified RNA
  source 1..76: mol_type = other DNA
  modified_base 1..76: mod_base = OTHER; note = phosphodiester linkage between nucleosides
gggacgctgacccagaagatctactatttctagtagatcttctgggtcagcgtccctatagtgagtcgtattacaa

SEQ ID NO: 67-69 - skipped (3 entries)

SEQ ID NO: 70 - DNA, 20 bp, synthetic construct
  misc_feature 1..20: note = Modified RNA
  source 1..20: mol_type = other DNA
  modified_base 1..20: mod_base = OTHER; note = phosphodiester linkage between nucleosides
cacacacacacacacacaca

SEQ ID NO: 71 - RNA, 20 nt, synthetic construct
  misc_feature 1..20: note = Modified RNA
  source 1..20: mol_type = other RNA
  modified_base 1..20: mod_base = OTHER; note = phosphodiester linkage between nucleosides
ggttgttgttgttgttgttg

SEQ ID NO: 72 - RNA, 20 nt, synthetic construct
  misc_feature 1..20: note = Modified RNA
  source 1..20: mol_type = other RNA
  modified_base 1..20: mod_base = OTHER; note = phosphorothioate linkage between nucleosides
ggttgttgttgttgttgttg